(19) United States
(12) Patent Application Publication
(10) Pub. No.: US 2022/0227873 A1
(43) Pub. Date: Jul. 21, 2022

(54) ANTI-GAL9 IMMUNE-INHIBITING BINDING MOLECULES

(71) Applicant: The Council of the Queensland Institute of Medical Research, Herston (AU)

(72) Inventors: Michelle WYKES, Herston (AU); Dileep K. PULUKKUNAT, Madison, WI (US)

(21) Appl. No.: 17/614,460

(22) PCT Filed: May 29, 2020
(86) PCT No.: PCT/AU2020/050546

§ 371 Date: Nov. 26, 2021

Related U.S. Application Data
(60) Provisional application No. 62/900,105, filed on Sep. 13, 2019.
(60) Provisional application No. 62/855,590, filed on May 31, 2019.

(51) Int. Cl. C07K 16/28

(52) U.S. Cl. CPC C07K 16/2851; A61P 37/06; C07K 16/2827

(57) ABSTRACT

Inhibitory anti-GAL9 binding molecules, antibody constructs, pharmaceutical compositions comprising the binding C molecules and antibody constructs, and methods of use thereof are presented.

### 1. CROSS-REFERENCE TO RELATED APPLICATIONS

This application claims the benefit under 35 U.S.C. 119(e) of prior co-pending U.S. Provisional Patent Application No. 62/900,105, filed on Sep. 13, 2019 and U.S. Provisional Patent Application No. 62/855,590, filed on May 31, 2019.

### 2. SEQUENCE LISTING

The instant application contains a Sequence Listing which has been submitted via EFS-Web and is hereby incorporated herein by reference in its entirety. Said ASCII copy, created on Month XX, 2020, is named XXXXXUS_sequencelisting.txt, and is X,XXX,XXX bytes in size.

### 3. BACKGROUND

Autoimmune diseases arise from an imbalance within the immune system that results in immune-mediated attack on the body's own cells and tissues. The current “gold standard” of care for autoimmune diseases is systemic immune suppression by immunosuppressive agents, including corticosteroids, anti-cytokine antibodies such as anti-TNF-α, anti-IL-1, anti-IL-5, anti-IL-6, anti-IL-17 antibodies, and anti-IL-23 antibodies, and small molecule drugs that reduce inflammatory cytokine signaling, such as JAK/STAT inhibitors. However, nonspecific systemic immune suppression predisposes the patient to infectious disease and can have other serious side effects.

Immune therapy has great potential for the treatment of autoimmune disease. Galectin-9 (GAL9) is an S-type lectin beta-galacto side-binding protein with N- and C-terminal carbohydrate-binding domains connected by a linker peptide. GAL9 has been implicated in modulating cell-cell and cell-matrix interactions. GAL9 has been shown to bind soluble PD-L2, and at least some of the immunological effects of PD-L2 have been suggested to be mediated through binding of multimeric PD-L2 to GAL9, rather than through PD-1 (WO 2016/008005, which is incorporated herein by reference in its entirety). However, mechanisms by which GAL9 and PD-L2 impact immune effector function are not yet fully characterized.

There remains a need for more targeted therapies that can reestablish balance of the immune system by modulating immune effector cells to establish a more clinically favorable cytokine profile. Such therapeutic agents may be useful for improving treatment for autoimmune and inflammatory disease.

### 4. SUMMARY

The present invention has arisen in part from the unexpected discovery that PD-L2 is overexpressed in autoimmune disease and that inhibiting the Galectin-9/PD-L2 pathway modulates immune effector cells to produce a more clinically favorable cytokine profile.

Accordingly, disclosed herein are various GAL9 binding molecules, antigen binding portions thereof, and antibodies that specifically bind to and antagonize human GAL9 (Galectin-9). Inhibiting GAL9 using the anti-human GAL9 binding molecules disclosed herein decreases the secretion and production of proinflammatory cytokines, increases the secretion and production of anti-inflammatory cytokines, and decreases surface expression of stimulatory molecules.

Pharmaceutical compositions comprising the GAL9 binding molecules are also disclosed. The anti-GAL9 binding molecules, antigen binding portions thereof, and antibodies disclosed herein can be used per se, as a pharmaceutical composition, or in combination with other therapeutic agents or procedures to treat, prevent, and/or diagnose autoimmune disease, inflammatory disease, or a condition that invokes an inflammation response such as an infection. The anti-GAL9 binding molecules are particularly useful for a disease or condition in which GAL9/PD-L2 interaction contributes prominently to pathogenesis. The anti-GAL9 binding molecules are useful in treating, reducing inflammation, reducing autoimmune response, prolonging remission, inducing remission, re-establishing immune tolerance, improving organ function, reducing progression of a disease, reducing the risk of development of a second disease, or increasing overall survival in a subject.

In a first aspect, the disclosure provides a Galectin-9 (GAL9) antigen binding molecule comprising a first antigen binding site specific (ABS) for a first epitope of a first GAL9 antigen, wherein the first antigen binding site comprises all three VH CDRs from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

In a second aspect, the disclosure provides a Galectin-9 (GAL9) antigen binding molecule, comprising a first antigen binding site specific for a first epitope of a first GAL9 antigen, wherein the first antigen binding site comprises all three VL CDRs from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

In a third aspect, the disclosure provides a Galectin-9 (GAL9) antigen binding molecule, comprising a first antigen binding site specific for a first epitope of a first GAL9 antigen, wherein the first antigen binding site comprises all three VH CDRs and all three VL CDRs from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

In a fourth aspect, the disclosure provides a Galectin-9 (GAL9) antigen binding molecule, comprising a first antigen binding site specific for a first epitope of a first GAL9 antigen, comprising the VL sequence and the VH sequence from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

In some embodiments, the GAL9 antigen binding molecule comprises a full immunoglobulin heavy chain “IgG1” sequence comprising the VH sequence and a full immunoglobulin light chain sequence comprising the VL sequence, wherein the VH sequence and the VL sequence are from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

In some embodiments, the GAL9 antigen binding molecule comprises a full immunoglobulin heavy chain “IgG4” sequence comprising the VH sequence and a full immunoglobulin light chain sequence comprising the VL sequence, wherein the VH sequence and the VL sequence are from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

In some embodiments, the GAL9 antigen binding molecule can comprise a GAL9 antigen that is a human GAL9 antigen.

In some embodiments, the GAL9 antigen binding molecule can further comprises a second antigen binding site.

In certain embodiments, the second antigen binding site is specific for the GAL9 antigen. In other embodiments, the second antigen binding site is identical to the first antigen binding site.

In other embodiments, the second antigen binding site is specific for a second epitope of the first GAL9 antigen.

In some embodiments, the second antigen binding site comprises all three VH CDRs, all three VL CDRs, or all three VH CDRs and all three VL CDRs from another ABS clone selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

In some embodiments, the second antigen binding site comprises the VL sequence and the VH sequence from the other ABS clone.

In some embodiments, the second antigen binding site comprises a full immunoglobulin heavy chain sequence comprising the VH sequence and a full immunoglobulin light chain sequence comprising the VL sequence from the other ABS clone.

In some embodiments, the second antigen binding site is specific for an antigen other than the first GAL9 antigen.

In some embodiments, the first antigen binding site comprises all three VH CDRs, all three VL CDRs, or all three VH CDRs and all three VL CDRs from any one of the ABS clones selected from: P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

In some embodiments, the first antigen binding site comprises all three VH CDRs, all three VL CDRs, or all three VH CDRs and all three VL CDRs from any one of the ABS clones selected from: P9-11, P9-24, P9-34, and P9-37.

In some embodiments, the first antigen binding site comprises all three VH CDRs, all three VL CDRs, or all three VH CDRs and all three VL CDRs from any one of the ABS clones selected from: P9-11, P9-24, and P9-34.

In some embodiments the first antigen binding site comprises all three VH CDRs, all three VL CDRs, or all three VH CDRs and all three VL CDRs from ABS clone P9-11.

In some embodiments, the first antigen binding site comprises all three VH CDRs, all three VL CDRs, or all three VH CDRs and all three VL CDRs from ABS clone P9-24.

In some embodiments, the first antigen binding site comprises all three VH CDRs, all three VL CDRs, or all three VH CDRs and all three VL CDRs from ABS clone P9-34.

In some embodiments, the first antigen binding site comprises all three VH CDRs, all three VL CDRs, or all three VH CDRs and all three VL CDRs from ABS clone P9-37.

In some embodiments, the GAL9 antigen binding molecule comprises an antibody format selected from the group consisting of: full-length antibodies, Fab fragments, F(ab)′2 fragments, Fvs, scFvs, tandem scFvs, diabodies, scDiabodies, DARTs, single chain VHH camelid antibodies, tandAbs, minibodies, and B-bodies. B-bodies are described in US pre-grant publication number US 2018/0118811, which is incorporated herein by reference in its entirety.

In some embodiments, the GAL9 antigen binding molecule decreases TNF-α secretion by activated immune cells upon contact, wherein the decrease is about at least a 30%, 35%, 40%, 45%, 50%, 55%, or 60% decrease relative to activated immune cells treated with a control agent.

In some embodiments, the GAL9 antigen binding molecule decreases IFN-γ secretion by activated immune cells upon contact, wherein the decrease is about at least a 20%, 25%, 30%, 35%, 40%, 45%, or 50% decrease relative to activated immune cells treated with a control agent.

In some embodiments, the GAL9 antigen binding molecule increases IL-10 secretion by activated immune cells upon contact, wherein the increase is about at least a 5%, 10%, 15%, 20%, 25%, 30%, 35% or 40% increase relative to activated immune cells treated with a control agent.

In some embodiments, the GAL9 antigen binding molecule does not modulate PD-1 surface expression on activated immune cells relative to activated immune cells treated with a control agent.

In some embodiments, the GAL9 antigen binding molecule does not modulate PD-L1 surface expression on activated immune cells relative to activated immune cells treated with a control agent.

In some embodiments, the GAL9 antigen binding molecule does not modulate CTLA-4 surface expression on activated immune cells relative to activated immune cells treated with a control agent.

In some embodiments, the GAL9 antigen binding molecule does not modulate TIM3 surface expression on activated immune cells relative to activated immune cells treated with a control agent.

In some embodiments, the GAL9 antigen binding molecule does not modulate LAG3 surface expression on activated immune cells relative to activated immune cells treated with a control agent.

In some embodiments, the GAL9 antigen binding molecule decreases 4-1BB surface expression on activated CD8⁺ T-cells, relative to activated CD8⁺ T-cells treated with a control agent.

In some embodiments, the GAL9 antigen binding molecule decreases CD40L surface expression on activated CD8⁺ T-cells, relative to activated CD8⁺ T-cells treated with a control agent.

In some embodiments, the GAL9 antigen binding molecule decreases OX40 surface expression activated on CD8⁺ T-cells, relative to activated CD8⁺ T-cells treated with a control agent.

In some embodiments, the control agent is a negative control agent or positive control agent.

In some embodiments, the control agent is a control antibody.

In some embodiments, the control antibody is selected from the group consisting of: an ECA42 clone anti-GAL9 antibody, an RG9.1 clone anti-GAL9 antibody, an RG9.35 clone anti GAL9 antibody, an anti-PD1 antibody, an 108A2 clone anti-GAL9 antibody, and a non-GAL9 binding isotype control antibody.

In some embodiments, the activated immune cells, activated CD8⁺ T-cells, or activated DCs were activated by were activated by peptide stimulation, anti-CD3, or dendritic cells.

In a fifth aspect, the disclosure provides a GAL9 antigen binding molecule that decreases TNF-α secretion by activated immune cells, wherein the decrease is about at least a 30%, 35%, 40%, 45%, 50%, 55%, or 60% decrease relative to activated immune cells treated with a control agent.

In a sixth aspect, the disclosure provides a GAL9 antigen binding molecule that decreases IFN-γ secretion by activated immune cells, wherein the decrease is about at least a 20%, 25%, 30%, 35%, 40%, 45%, or 50% decrease relative to activated immune cells treated with a control agent.

In a seventh aspect, the disclosure provides a GAL9 antigen binding molecule that increases IL-10 secretion by activated immune cells, wherein the increase is about at least a 5%, 10%, 15%, 20%, 25%, 30%, 35%, or 40% increase relative to activated immune cells treated with a control agent

In an eighth aspect, the disclosure provides a GAL9 antigen binding molecule does not modulate PD-1 surface expression on activated immune cells relative to activated immune cells treated with a control agent.

In a ninth aspect, the disclosure provides a GAL9 antigen binding molecule does not modulate PD-L1 surface expression on activated immune cells relative to activated immune cells treated with a control agent.

In a tenth aspect, the disclosure provides a GAL9 antigen binding molecule does not modulate CTLA-4 surface expression on activated immune cells relative to activated immune cells treated with a control agent.

In an eleventh aspect, the disclosure provides a GAL9 antigen binding molecule does not modulate TIM3 surface expression on activated immune cells relative to activated immune cells treated with a control agent.

In a twelfth aspect, the disclosure provides a GAL9 antigen binding molecule does not modulate LAG3 surface expression on activated immune cells relative to activated immune cells treated with a control agent.

In a thirteenth aspect, the disclosure provides a GAL9 antigen binding molecule decreases 4-1BB surface expression on activated CD8⁺ T-cells, relative to activated CD8⁺ T-cells treated with a control agent.

In a fourteenth aspect, the disclosure provides a GAL9 antigen binding molecule decreases CD40L surface expression on activated CD8⁺ T-cells, relative to activated CD8⁺ T-cells treated with a control agent.

In a fifteenth aspect, the disclosure provides a GAL9 antigen binding molecule decreases OX40 surface expression on activated CD8⁺ T-cells, relative to activated CD8⁺ T-cells treated with a control agent.

In a sixteenth aspect, the disclosure provides a GAL9 antigen binding molecule demonstrates one or more of the following properties: A) decreases TNF-α secretion by activated immune cells, wherein the decrease is about at least a 30%, 35%, 40%, 45%, 50%, 55%, or 60% decrease relative to activated immune cells treated with a control agent; B) decreases IFN-γ secretion by activated immune cells, wherein the decrease is about at least a 20%, 25%, 30%, 35%, 40%, 45%, or 50% decrease relative to activated immune cells treated with a control agent; C) increases IL-10 secretion by activated immune cells, wherein the increase is about at least a 5%, 10%, 15%, 20%, 25%, 30%, 35%, or 40% increase relative to activated immune cells treated with a control agent; D) does not modulate PD-1 surface expression on activated immune cells relative to activated immune cells treated with a control agent; E) does not modulate PD-L1 surface expression on activated immune cells relative to activated immune cells treated with a control agent; F) does not modulate CTLA-4 surface expression on activated immune cells relative to activated immune cells treated with a control agent; G) does not modulate TIM3 surface expression on activated immune cells relative to activated immune cells treated with a control agent; H) does not modulate LAG3 surface expression on activated immune cells relative to activated immune cells treated with a control agent; I) decreases 4-1BB surface expression on activated CD8⁺ T-cells, relative to activated CD8⁺ T-cells treated with a control agent; J); decreases CD40L surface expression on activated CD8⁺ T-cells, relative to activated CD8⁺ T-cells treated with a control agent; or K) decreases OX40 surface expression on activated CD8⁺ T-cells, relative to activated CD8⁺ T-cells treated with a control agent.

In some embodiments, the control agent is a negative control agent or positive control agent.

In some embodiments, the control agent is a control antibody.

In some embodiments, the control antibody is selected from the group consisting of: an ECA42 clone anti-GAL9 antibody, an RG9.1 clone anti-GAL9 antibody, an RG9.35 clone anti GAL9 antibody, an anti-PD1 antibody, an 108A2 clone anti-GAL9 antibody, and an non-GAL9 binding isotype control antibody.

In some embodiments, the activated immune cells, were activated by were activated by peptide stimulation, anti-CD3 or dendritic cells.

In some embodiments, the GAL9 antigen binding molecule of the fifth-fifteenth aspects provided herein comprise a first antigen binding site specific for a first epitope of a first GAL9 antigen, wherein the first antigen binding site comprises all three VH CDRs and all three VL CDRs from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9- 57.

In some embodiments, the VL sequence and the VH sequence from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

In some certain embodiments, the GAL9 antigen binding molecule comprises a full immunoglobulin heavy chain sequence comprising the VH sequence and a full immunoglobulin light chain sequence comprising the VL sequence, wherein the VH sequence and the VL sequence are from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

In some embodiments, the GAL9 antigen is a human GAL9 antigen.

In some embodiments, the GAL9 antigen binding molecule further comprises a second antigen binding site.

In some embodiments, the second antigen binding site is specific for the GAL9 antigen.

In some embodiments, the second antigen binding site is identical to the first antigen binding site.

In some embodiments, the second antigen binding site is specific for a second epitope of the first GAL9 antigen.

In some embodiments, the second antigen binding site comprises all three VH CDRs and all three VL CDRs from another ABS clone selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

In some embodiments, the second antigen binding site comprises the VL sequence and the VH sequence from the other ABS clone.

In some embodiments, the second antigen binding site comprises a full immunoglobulin heavy chain sequence comprising the VH sequence and a full immunoglobulin light chain sequence comprising the VL sequence from the other ABS clone.

In some embodiments, the second antigen binding site is specific for an antigen other than the first GAL9 antigen.

In some embodiments, the first antigen binding site comprises all three VH CDRs and all three VL CDRs from any one of the ABS clones selected from: P9-11, P9-24, P9-34, and P9-37.

In some embodiments, the first antigen binding site comprises all three VH CDRs and all three VL CDRs from any one of the ABS clones selected from: P9-11, P9-24, and P9-34.

In some embodiments, the first antigen binding site comprises all three VH CDRs and all three VL CDRs from ABS clone P9-11.

In some embodiments, the first antigen binding site comprises all three VH CDRs and all three VL CDRs from ABS clone P9-24.

In some embodiments, the first antigen binding site comprises all three VH CDRs and all three VL CDRs from ABS clone P9-34.

In some embodiments, the first antigen binding site comprises all three VH CDRs and all three VL CDRs from ABS clone P9-37.

In some embodiments, the GAL9 antigen binding molecule comprises an antibody format selected from the group consisting of: full-length antibodies, Fab fragments, Fvs, scFvs, tandem scFvs, Diabodies, scDiabodies, DARTs, tandAbs, minibodies, and B-bodies.

In a seventeenth aspect, the disclosure provides a GAL9 antigen binding molecule which binds to the same epitope as a GAL9 antigen binding molecule of any one of the preceding claims.

In an eighteenth aspect, the disclosure provides a GAL9 antigen binding molecule which competes for binding with a GAL9 antigen binding molecule of any one of the preceding claims.

In some embodiments, the GAL9 antigen binding molecule is purified.

In a nineteenth aspect, the disclosure provides a pharmaceutical composition comprising the GAL9 antigen binding molecule of any one of the preceding claims and a pharmaceutically acceptable diluent.

In a twentieth aspect, the disclosure provides a method for treating a subject with an autoimmune disease comprising administering a therapeutically effective amount of the pharmaceutical composition as provided herein to the subject.

In some embodiments, the subject with an autoimmune disease has increased expression of PD-L2 on dendritic cells, as compared to dendritic cells from a healthy control.

In some embodiments, the autoimmune disease is selected from the group consisting of: inflammatory bowel disease, Crohn's disease, ulcerative colitis, colitis, celiac disease, rheumatoid arthritis, Behçet's disease, amyloidosis, psoriasis, psoriatic arthritis, systemic lupus erythematosus nephritis, graft-versus-host disease (GVHD), nonalcoholic steatohepatitis (NASH), and ankylosing spondylitis.

In some embodiments, administering a therapeutically effective amount of the GAL binding molecule per se or a pharmaceutical composition results in reducing inflammation, reducing autoimmune response, prolonging remission, inducing remission, re-establishing immune tolerance, improving organ function, reducing the progression of a disease, reducing the risk of progression or development of a second disease, or increasing overall survival.

### 5. BRIEF DESCRIPTION OF THE DRAWINGS

FIGS. 1A and 1B show an illustrative example of various CDR and framework numbering systems—Chothia, Martin (ABA), and Kabat—as applied to the P9-01 anti-human Gal9 candidate antibody provided herein.

FIG. 2 shows density contour plots of the percentage of CD11c⁺ blood dendritic cells from a Crohn's Disease patient detected as positive for PD-L1 or PD-L2 expression compared to labelling isotype IgG control.

FIGS. 3A and 3B show scatter plots of the percentage of PD-L1 or PD-L2 expressing blood dendritic cells in healthy controls or Crohn's Disease patients. FIGS. 3C and 3D show scatter plots of the Geometric Mean Fluorescence (GMI) of PD-L1 or PD-L2 surface expression on blood dendritic cells in healthy controls or Crohn's Disease patients.

FIGS. 4A and 4B show representative confocal images of DNA (DAPI; blue), PD-L1 (green), and PD-L2 (red) expression on dendritic cells from two healthy control donors (4A) and three Crohn's Disease patients (4B); rendered in gray scale in the attached figures.

FIGS. 5A-5C show the mean concentration of cytokines secreted by PMBCs from Crohn's Disease (CD) patients after treatment with anti-CD3 to mimic TCR activation and either anti-PD-L2 (αPD-L2) or IgG control. FIGS. 5A-5B show the mean concentration of TNF-α and IFN-γ after treatment with anti-PD-L2 or IgG control in PMBCs from CD patients. FIG. 5C shows the mean ratio of IL-10:TNF-α secretion after treatment with anti-PD-L2 and IgG control in PMBCs from CD patients.

FIG. 6 shows TNF-α secretion by anti-CD3 activated mouse CD4⁺ T-cells after treatment with either sPD-L2 or both sPD-L2 and inhibitory anti-mouse anti-GAL9 (108A2).

FIG. 7 shows representative confocal images of DNA (DAPI; blue), PD-L1 (green), PD-1 (red) and OX40 (yellow) expression in CD4⁺ T-cells from malaria-infected mice after treatment with mouse inhibitory anti-mouse GAL9 (108A2) and activating anti-mouse GAL9 (RG9.1) antibodies; rendered in gray scale in the attached figures.

FIGS. 8A and 8B show bar graphs of the percentage of surviving mouse CD4⁺ and CD8⁺ T-cells after treatment with either sPD-L2 or sPD-L2 and mouse inhibitory anti-GAL9 (108A2) antibody.

FIGS. 9A and 9B show bar graphs of INF-γ (9A) and TNF-α (9B) secretion from mouse CD4⁺ T-cells co-cultured with dendritic cells (stimulated) and treated with either blocking anti-PD-L2 (clone Ty25) or inhibitory anti-GAL9 (108A2) mouse antibodies, compared to control, unstimulated CD4⁺ T-cells.

FIGS. 10A and 10B show INF-γ (10A) and TNF-α (10B) secretion from HCMV peptide, in vitro-stimulated PBMCs after treatment with various anti-human GAL9 candidates, a known activating tool antibody (Tool mAb), an anti-PD-1 antibody, a IgG control antibody (IgG Ctrl), and a vehicle control (PBS Ctrl). Black diamond shapes show secretion from activated PBMCs stimulated by Tool mAb and anti-PD-1 antibody.

FIGS. 11A-11C show INF-γ and TNF-α secretion from HCMV peptide, in vitro-stimulated PBMCs after treatment with anti-human GAL9 P9-11, P9-37, or P9-57 compared to IgG control antibody (IgG).

FIGS. 12A-12C show TNF-α (12A), INF-γ (12B), and IL-10 (12C) secretion from HCMV peptide, in vitro-stimulated PBMCs after treatment with anti-human GAL9 candidates P9-11, P9-24, or P9-34 compared to IgG control antibody (IgG).

FIGS. 13A and 13B show bar graphs of the ratio of TNF-α:IL-10 secretion (13A) and ratio of IFN-γ:IL-10 secretion (13B) from anti-CD3 activated mouse CD3⁺ T-cells after treatment with inhibitory anti-mouse GAL9 (108A2) and anti-human GAL9 P9-11, P9-24, or P9-34.

### 6. DETAILED DESCRIPTION

### 6.1. Definitions

Unless defined otherwise, all technical and scientific terms used herein have the meaning commonly understood by a person skilled in the art to which this invention belongs. As used herein, the following terms have the meanings ascribed to them below.

By “antigen binding site” or “ABS” is meant a region of a GAL9 binding molecule that specifically recognizes or binds to a given antigen or epitope.

As used herein, the terms “treat” or “treatment” are used in their broadest accepted clinical sense. The terms include, without limitation, lessening a sign or symptom of disease; improving a sign or symptom of disease; alleviation of symptoms; diminishment of extent of disease; stabilized (i.e., not worsening) state of disease; delay or slowing of disease progression; amelioration or palliation of the disease state; remission (whether partial or total), whether detectable or undetectable; cure; prolonging survival as compared to expected survival if not receiving treatment. Unless explicitly stated otherwise, “treat” or “treatment” do not intend prophylaxis or prevention of disease.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on. Unless otherwise stated, “patient” intends a human “subject.”

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease.

The term “prophylactically effective amount” is an amount that is effective to prevent a symptom of a disease.

6.2. Other Interpretational Conventions

Unless otherwise specified, all references to sequences herein are to amino acid sequences.

Unless otherwise specified, antibody constant region residue numbering is according to the Eu index as described at www.imgt.org/IMGTScientificChart/Numbering/Hu_IGHGnber.html#refs (accessed Aug. 22, 2017), which is hereby incorporated by reference in its entirety, and residue numbers identify the residue according to its location in an endogenous constant region sequence regardless of the residue's physical location within a chain of the GALS binding molecules described herein.

Unless otherwise specified as “Kabat CDR”, “Chothia CDR”, “Contact CDR”, or “IMGT CDR”, all references to “CDRs” are to CDRs defined using the Martin (ABA) definition.

By “endogenous sequence” or “native sequence” is meant any sequence, including both nucleic acid and amino acid sequences, which originates from an organism, tissue, or cell and has not been artificially modified or mutated.

Polypeptide chain numbers (e.g., a “first” polypeptide chains, a “second” polypeptide chain. Etc. or polypeptide “chain 1,” “chain 2,” etc.) are used herein as a unique identifier for specific polypeptide chains that form a binding molecule and is not intended to connote order or quantity of the different polypeptide chains within the binding molecule.

In this disclosure, “comprises,” “comprising,” “containing,” “having,” “includes,” “including,” and linguistic variants thereof have the meaning ascribed to them in U.S. Patent law, permitting the presence of additional components beyond those explicitly recited.

As used herein, the singular forms “a,” “an,” and “the” include the plural referents unless the context clearly indicates otherwise. The terms “include,” “such as,” and the like are intended to convey inclusion without limitation, unless otherwise specifically indicated.

Ranges provided herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

6.3. General Overview

The present disclosure provides Galectin-9 (GAL9) antigen binding molecules, such as anti-GAL9 antibodies and antigen-binding fragments thereof; compositions comprising the GAL9-binding molecules; pharmaceutical compositions comprising the GAL9-binding molecules; and methods of using the GAL9 binding molecules to treat subjects with a disease or a condition. The disclosure particularly provides various GAL9 antigen binding molecules that are inhibitory, acting as inhibitors of the immune system, decreasing the secretion and production of pro-inflammatory cytokines and increasing the secretion and production of anti-inflammatory cytokines in various immune cells and decreasing surface expression of stimulatory molecules.

The GAL9 antigen binding molecules are particularly useful for the treatment of an autoimmune disease or inflammatory disease in a subject. In some embodiments, the compositions and methods are used to treat an infection that causes an inflammatory response in a subject. The anti-GAL9 binding molecules are particularly useful for treating a disease or condition in which GAL9/PD-L2 interaction contributes prominently to pathogenesis. In some embodiments, the anti-GAL9 binding molecules are administered to a subject per se, as a pharmaceutical composition, or in combination with other therapeutic agents or procedures.

6.4. GAL9 Antigen Binding Molecules

In a first aspect, antigen binding molecules are provided. In every embodiment, the antigen binding molecule includes at least a first antigen binding site specific for a GAL9 antigen; the binding molecules are therefore termed GAL9 antigen binding molecules or GAL9 binding molecules.

The GAL9 antigen binding molecules described herein bind specifically to GAL9 antigens.

As used herein, “GAL9 antigens” refer to Galectin-9 family members and homologs. GAL9 is also referred to as LGALS9, HUAT, LGALS9A, tumor antigen HOM-HD-21, and ecalectin. In particular embodiments, the GAL9 binding molecule has antigen binding sites that specifically bind to at least a portion of more than one GAL9 domain, such as the junction between a first and a second GAL9 domain.

In specific embodiments, the GAL9 antigen is human. GenBank Accession #NP_033665.1 describes a canonical human GAL9 protein, including its sequences and domain features, and is hereby incorporated by reference in its entirety. SEQ ID NO:6 provides the full-length GAL9 protein sequence.

[SEQ ID NO: 6]
MAFSGSQAPYLSPAVPFSGTIQGGLQDGLQITVNGTVLSSSGTRFAVNF
QTGFSGNDIAFHFNPRFEDGGYVVCNTRQNGSWGPEERKTHMPFQKGMP
FDLCFLVQSSDFKVMVNGILFVQYFHRVPFHRVDTISVNGSVQLSYISF
QNPRTVPVQPAFSTVPFSQPVCFPPRPRGRRQKPPGVWPANPAPITQTV
IHTVQSAPGQMFSTPAIPPMMYPHPAYPMPFITTILGGLYPSKSILLSG
TVLPSAQRFHINLCSGNHIAFHLNPRFDENAVVRNTQIDNSWGSEERSL
PRKMPFVRGQSFSVWILCEAHCLKVAVDGQHLFEYYHRLRNLPTINRLE
VGGDIQLTHVQT

In various embodiments, the GAL9 binding molecule additionally binds specifically to at least one antigen additional to a GAL9 antigen.

6.4.1. Functional Characteristics of the GAL9 Antigen Binding Molecules

In typical embodiments, upon contact therewith, the GAL9 antigen binding molecule modulates cytokine secretion (e.g., increases or decreases cytokine secretion) of immune cells or activated immune cells. In some embodiments, the immune cells are peripheral blood mononuclear cells (PBMCs). In some embodiments, the immune cells are T cells. In some embodiments, the T cells are effector T cells. In some embodiments, the T cells are CD8⁺ T cells. In embodiments, the T cells are CD4⁺ T cells. In some embodiments, the T cells are CD3⁺ T cells.

The impact of the GAL9 antigen binding molecule on immune cell cytokine secretion may be determined by any suitable means. For instance, the impact of the GAL9 antigen binding molecule on immune cell cytokine secretion may be determined in vivo, ex vivo, or in vitro. In some embodiments, cytokine secretion is determined in activated immune cells contacted with a GAL9 antigen binding molecule, as compared to activated immune cells contacted with a control agent, e.g., a control antigen binding molecule or vehicle control. The immune cells may be activated by peptide stimulation. For example, the immune cells may be activated by a peptide or plurality of peptides known to induce an immune response. A plurality of peptides known to induce an immune response can be from an infection from a pathogen such as a viral infection or bacterial infection.

The control agent can be a negative control or a positive control. In some embodiments, the GAL9 antigen binding molecule increases cytokine secretion in immune cells, relative to a negative control agent or negative control antigen binding molecule. In some embodiments, the negative control antigen binding molecule is an isotype control binding molecule that does not bind GAL9. In some embodiments, the positive control antibody is an anti-PD1 antibody, such as nivolumab. In some embodiments, the positive control antibody is a GAL9 control antibody. The GAL9 control antibody can be Gal9 antibody clone RG9.1 (Cat. No. BE0218, InVivoMab Antibodies) or RG9.35. RG9.1 and RG9.35 are both described in Fukushima A, Sumi T, Fukuda K, Kumagai N, Nishida T, et al. (2008), which is incorporated herein by reference in its entirety. Roles of galectin-9 in the development of experimental allergic conjunctivitis in mice. Int Arch Allergy Immunol 146: 36-43, which is hereby incorporated by reference in its entirety. The GAL9 control antibody can be GAL9 antibody clone ECA42 (Cat. No. LS-C179449, LifeSpan BioScience). The GAL9 control antibody can be GAL9 antibody clone 108A2 (BioLegend® San Diego, Calif.). In some embodiments, the GAL9 antigen binding molecule decreases cytokine secretion of proinflammatory cytokine in immune cells, relative to a control antibody. In some embodiments, the GAL9 antigen binding molecule increases cytokine secretion of inhibitory cytokine in immune cells, relative to a control antibody.

Cytokine secretion by the immune cells can be assessed by any suitable means. By way of example only, cytokine secretion by in vitro or ex vivo immune cell culture models may be assessed by analyzing cytokine content of the cultured cell supernatants, e.g., by cytokine bead array.

In some embodiments, the cytokine is TNF-α. In some embodiments, the GAL9 antigen binding molecule decreases TNF-α secretion in activated immune cells by at least 1%, 5%, 10%, 15%, 20%, 25%, 30%, 35%, 40%, 45%, 50%, 55%, 60%, 65%, 70%, 75%, 80%, 85%, or 90%, as compared to a control agent described herein. In some embodiments, the GAL9 antigen binding molecule decreases TNF-α secretion in activated immune cells by at least 1%-5%, 5-10%, 10-15%, 15-20%, 20-25%, 25-30%, 30-35%, 35%-40%, 40%-45%, 45%-50%, 50%-55%, 55%-60%, 60%-65%, 70%-75%, 75%-80%, 80%-85%, or 85%-90% decrease, as compared to a control agent described herein. In some embodiments, the GAL9 antigen binding molecule decreases TNF-α secretion in activated immune cells by about 30%-50% decrease, as compared to a control agent described herein.

In some embodiments, the cytokine is IFN-γ. In some embodiments, the GAL9 antigen binding molecule decreases IFN-γ secretion in activated immune cells by at least at least 1%, 5%, 10%, 15%, 20%, 25%, 30%, 35%, 40%, 45%, 50%, 55%, 60%, 65%, 70%, or 75% as compared to a control agent described herein. In some embodiments, the GAL9 antigen binding molecule decreases IFN-γ secretion in activated immune cells by at least 10-15%, 15-20%, 20-25%, 25-30%, 30-35%, 35%-40%, 40%-45%, 45%-50%, 50%-55%, 55%-60%, 60%-65%, or 70%-75% decrease, as compared to a control agent described herein. In some embodiments, the GAL9 antigen binding molecule decreases IFN-γ secretion in activated immune cells by about 20%-40% decrease, as compared to a control agent described herein.

In some embodiments, the cytokine is IL-10. In some embodiments, the GAL9 antigen binding molecule increases IL-10 secretion in activated immune cells by at least 1%, 5%, 10%, 15%, 20%, 25%, 30%, 35%, 40%, 45%, or 50% increase, as compared to a control agent described herein. In some embodiments, the GAL9 antigen binding molecule increases IL-10 secretion in activated immune cells by at least 1%-5%, 5%-10%, 10-15%, 15-20%, 20-25%, 25-30%, 30-35%, 35%-40%, 40%-45%, or 45%-50% increase, as compared to a control agent described herein. In some embodiments, the GAL9 antigen binding molecule increases IL-10 secretion in activated immune cells by about 5%-30% increase, as compared to a control agent described herein.

In some embodiments, upon contact therewith, the GAL9 antigen binding molecule does not modulate surface expression of immune checkpoint molecule(s) (e.g., stimulatory or inhibitory checkpoint molecules) relative to activated immune cells treated with a control agent. The term “does not modulate” means that there is no substantial increase or decrease in the expression of the immune checkpoint molecule after treatment with a GAL9 binding molecule provided herein, compared to a control agent. In some embodiments, no substantial increase in surface expression (e.g., does not modulate expression) is an increase of cell surface expression that is no more than 1.01×, 1.02×, 1.03×, 1.04×, 1.05×, 1.06×, 1.07×, 1.08×, 1.09×, 1.1×, 1.2×, or 1.3× fold change, relative to activated immune cells treated with a control agent. In some embodiments, no substantial decrease in surface expression (e.g., does not modulate expression) is a decrease of cell surface expression that is no more than 0.01×, 0.02×, 0.03×, 0.04×, 0.05×, 0.06×, 0.07×, 0.08×, 0.09×, 0.1×, or 0.2× fold change, relative to activated immune cells treated with a control agent.

In some embodiments, no substantial increase in surface expression (e.g., does not modulate expression) is an increase of surface expression about a 1% increase, 2% increase, 3% increase, 4% increase, 5% increase, 6% increase, 7% increase, 8% increase, 9% increase, 10% increase, 11% increase, 12% increase, 13% increase, 14% increase, or 15% increase, relative to activated immune cells treated with a control agent. In some embodiments, no substantial decrease in surface expression (e.g., does not modulate expression) is a decrease of surface expression about a 1% decrease, 2% decrease, 3% decrease, 4% decrease, 5% decrease, 6% decrease, 7% decrease, 8% decrease, 9% decrease, 10% decrease, 11% decrease, 12% decrease, 13% decrease, 14% decrease, or 15% decrease, relative to activated immune cells treated with a control agent.

In some embodiments, no substantial increase or decrease in surface expression is determined by comparing the level of surface expression to the level of noise in the assay (e.g., in vivo, ex vivo, or in vitro). In some embodiments, no substantial increase or decrease in surface expression is determined by comparing the level of surface expression to the standard deviation in the assay (e.g., in vivo, ex vivo, or in vitro).

The impact of the GAL9 antigen binding molecule on surface expression of the one or more immune checkpoint molecules may be determined by any suitable means. For instance, the impact of the GAL9 antigen binding molecule on surface expression of the one or more costimulatory molecules may be determined in vivo, ex vivo, or in vitro.

In some embodiments, one or more immune checkpoint molecules are selected from PD-1, PD-L1, CTLA-4, TIM3, LAG3, TIGIT, and PVRIG. In some embodiments, one or more checkpoint molecules is selected from PD-1, PD-L1, TIM3, and LAG3. In some embodiments, the immune checkpoint molecule is PD-1 or PD-L1. In various embodiments, the activated (e.g., stimulated) immune cells are T-cells, CD8⁺ T cells, CD4⁺ T cells, CD3⁺ T cells, or PBMCs.

In some embodiments, the immune checkpoint molecule is PD-1. In some embodiments, activated CD8⁺ or CD4⁺ T-cells treated with the GAL9 antigen binding molecule exhibits an increase that is no more than 1.01×, 1.02×, 1.03×, 1.04×, 1.05×, 1.06×, 1.07×, 1.08×, 1.09×, 1.1×, 1.2×, or 1.3× fold change in PD-1 surface expression, relative to activated CD4⁺ or CD8⁺ T-cells treated with a control agent. In some embodiments, activated CD8⁺ or CD4⁺ T-cells treated with the GAL9 antigen binding molecule exhibits a decrease in surface expression that is no more than 0.01×, 0.02×, 0.03×, 0.04×, 0.05×, 0.06×, 0.07×, 0.08×, 0.09×, 0.1×, or 0.2× fold change in PD-1 surface expression, relative to activated CD4⁺ or CD8⁺ T-cells treated with a control agent.

In some embodiments, activated CD8⁺ or CD4⁺ T-cells treated with the GAL9 antigen binding molecule exhibits an increase that is no more than about a 1% increase, 2% increase, 3% increase, 4% increase, 5% increase, 6% increase, 7% increase, 8% increase, 9% increase, 10% increase, 11% increase, 12% increase, 13% increase, 14% increase, or 15% increase in PD-1 surface expression, relative to activated CD4⁺ or CD8⁺ T-cells treated with a control agent. In some embodiments, activated CD8⁺ or CD4⁺ T-cells treated with the GAL9 antigen binding molecule exhibits an decrease that is no more than about a 1% decrease, 2% decrease, 3% decrease, 4% decrease, 5% decrease, 6% decrease, 7% decrease, 8% decrease, 9% decrease, 10% decrease, 11% decrease, 12% decrease, 13% decrease, 14% decrease, or 15% decrease in PD-1 surface expression, relative to activated CD4⁺ or CD8⁺ T-cells treated with a control agent.

In some embodiments, the immune checkpoint molecule is PD-L1. In some embodiments, activated CD8⁺ or CD4⁺ T-cells treated with the GAL9 antigen binding molecule exhibits an increase that is no more than fold change in PD-L1 surface expression, relative to activated CD4⁺ or CD8⁺ T-cells treated with a control agent. In some embodiments, activated CD8⁺ or CD4⁺ T-cells treated with the GAL9 antigen binding molecule exhibits an increase that is no more than 1.01×, 1.02×, 1.03×, 1.04×, 1.05×, 1.06×, 1.07×, 1.08×, 1.09×, 1.1×, 1.2×, or 1.3× fold change in PD-L1 surface expression, relative to activated CD4⁺ or CD8⁺ T-cells treated with a control agent. In some embodiments, activated CD8⁺ or CD4⁺ T-cells treated with the GAL9 antigen binding molecule exhibits a decrease in surface expression that is no more than 0.01×, 0.02×, 0.03×, 0.04×, 0.05×, 0.06×, 0.07×, 0.08×, 0.09×, 0.1×, or 0.2× fold change in PD-L1 surface expression, relative to activated CD4⁺ or CD8⁺ T-cells treated with a control agent.

In some embodiments, activated CD8⁺ or CD4⁺ T-cells treated with the GAL9 antigen binding molecule exhibit an increase that is no more than about a 1% increase, 2% increase, 3% increase, 4% increase, 5% increase, 6% increase, 7% increase, 8% increase, 9% increase, 10% increase, 11% increase, 12% increase, 13% increase, 14% increase, or 15% increase in PD-L1 surface expression relative to activated CD4⁺ or CD8⁺ T-cells treated with a control agent. In some embodiments, activated CD8⁺ or CD4⁺ T-cells treated with the GAL9 antigen binding molecule exhibits a decrease that is no more than about a 1% decrease, 2% decrease, 3% decrease, 4% decrease, 5% decrease, 6% decrease, 7% decrease, 8% decrease, 9% decrease, 10% decrease, 11% decrease, 12% decrease, 13% decrease, 14% decrease, or 15% decrease in PD-L1 surface expression, relative to activated CD4⁺ or CD8⁺ T-cells treated with a control agent.

In some embodiments, the immune checkpoint molecule is CTLA-4. In some embodiments, activated CD8⁺ or CD4⁺ T-cells treated with the GAL9 antigen binding molecule exhibits an increase that is no more than 1.01×, 1.02×, 1.03×, 1.04×, 1.05×, 1.06×, 1.07×, 1.08×, 1.09×, 1.1×, 1.2×, or 1.3× fold change in CTLA-4 surface expression, relative to activated CD4⁺ or CD8⁺ T-cells treated with a control agent. In some embodiments, activated CD8⁺ or CD4⁺ T-cells treated with the GAL9 antigen binding molecule exhibits a decrease in surface expression that is no more than 0.01×, 0.02×, 0.03×, 0.04×, 0.05×, 0.06×, 0.07×, 0.08×, 0.09×, 0.1×, or 0.2× fold change in CTLA-4 surface expression, relative to activated CD4⁺ or CD8⁺ T-cells treated with a control agent.

In some embodiments, activated CD8⁺ or CD4⁺ T-cells treated with the GAL9 antigen binding molecule exhibits an increase that is no more than about a 1% increase, 2% increase, 3% increase, 4% increase, 5% increase, 6% increase, 7% increase, 8% increase, 9% increase, 10% increase, 11% increase, 12% increase, 13% increase, 14% increase, or 15% increase in CTLA-4 surface expression, relative to activated CD4⁺ or CD8⁺ T-cells treated with a control agent. In some embodiments, activated CD8⁺ or CD4⁺ T-cells treated with the GAL9 antigen binding molecule exhibits a decrease that is no more than about a 1% decrease, 2% decrease, 3% decrease, 4% decrease, 5% decrease, 6% decrease, 7% decrease, 8% decrease, 9% decrease, 10% decrease, 11% decrease, 12% decrease, 13% decrease, 14% decrease, or 15% decrease in CTLA-4 surface expression, relative to activated CD4⁺ or CD8⁺ T-cells treated with a control agent.

In some embodiments, the immune checkpoint molecule is TIM3. In some embodiments, activated CD8⁺ or CD4⁺ T-cells treated with the GAL9 antigen binding molecule exhibits an increase that is no more than 1.01×, 1.02×, 1.03×, 1.04×, 1.05×, 1.06×, 1.07×, 1.08×, 1.09×, 1.1×, 1.2×, or 1.3× fold change in TIM3 surface expression, relative to activated CD4⁺ or CD8⁺ T-cells treated with a control agent. In some embodiments, activated CD8⁺ or CD4⁺ T-cells treated with the GAL9 antigen binding molecule exhibits a decrease in surface expression that is no more than 0.01×, 0.02×, 0.03×, 0.04×, 0.05×, 0.06×, 0.07×, 0.08×, 0.09×, 0.1×, or 0.2× fold change in TIM3 surface expression, relative to activated CD4⁺ or CD8⁺ T-cells treated with a control agent.

In some embodiments, activated CD8⁺ or CD4⁺ T-cells treated with the GAL9 antigen binding molecule exhibits an increase that is no more than about a 1% increase, 2% increase, 3% increase, 4% increase, 5% increase, 6% increase, 7% increase, 8% increase, 9% increase, 10% increase, 11% increase, 12% increase, 13% increase, 14% increase, or 15% increase in TIM3 surface expression, relative to activated CD4⁺ or CD8⁺ T-cells treated with a control agent. In some embodiments, activated CD8⁺ or CD4⁺ T-cells treated with the GAL9 antigen binding molecule exhibits a decrease that is no more than about a 1% decrease, 2% decrease, 3% decrease, 4% decrease, 5% decrease, 6% decrease, 7% decrease, 8% decrease, 9% decrease, 10% decrease, 11% decrease, 12% decrease, 13% decrease, 14% decrease, or 15% decrease in TIM3 surface expression, relative to activated CD4⁺ or CD8⁺ T-cells treated with a control agent.

In some embodiments, the immune checkpoint molecule is LAG3. In some embodiments, activated CD8⁺ or CD4⁺ T-cells treated with the GAL9 antigen binding molecule exhibits an increase that is no more than 1.01×, 1.02×, 1.03×, 1.04×, 1.05×, 1.06×, 1.07×, 1.08×, 1.09×, 1.1×, 1.2×, or 1.3× fold change in LAG3 surface expression, relative to activated CD4⁺ or CD8⁺ T-cells treated with a control agent. In some embodiments, activated CD8⁺ or CD4⁺ T-cells treated with the GAL9 antigen binding molecule exhibits a decrease in surface expression that is no more than 0.01×, 0.02×, 0.03×, 0.04×, 0.05×, 0.06×, 0.07×, 0.08×, 0.09×, 0.1×, or 0.2× fold change in LAG3 surface expression, relative to activated CD4⁺ or CD8⁺ T-cells treated with a control agent. In some embodiments, activated CD8⁺ or CD4⁺ T-cells treated with the GAL9 antigen binding molecule exhibits an increase that is no more than about a 1% increase, 2% increase, 3% increase, 4% increase, 5% increase, 6% increase, 7% increase, 8% increase, 9% increase, 10% increase, 11% increase, 12% increase, 13% increase, 14% increase, or 15% increase in LAG3 surface expression, relative to activated CD4⁺ or CD8⁺ T-cells treated with a control agent. In some embodiments, activated CD8⁺ or CD4⁺ T-cells treated with the GAL9 antigen binding molecule exhibits a decrease that is no more than about a 1% decrease, 2% decrease, 3% decrease, 4% decrease, 5% decrease, 6% decrease, 7% decrease, 8% decrease, 9% decrease, 10% decrease, 11% decrease, 12% decrease, 13% decrease, 14% decrease, or 15% decrease in LAG3 surface expression, relative activated to CD4⁺ or CD8⁺ T-cells treated with a control agent.

In some embodiments, the GAL9 antigen binding molecule decreases surface expression of one or more costimulatory molecules on immune cells, e.g., human immune cells. In certain embodiments, the GAL9 antigen binding molecule decreases surface expression of the one or more costimulatory molecules in activated immune cells. In particular embodiments, the activated immune cells are T cells. In specific embodiments, the activated immune cells are CD8⁺ T cells. In some embodiments, the one or more costimulatory molecules is selected from 4-1BB, CD40L, and OX40. In some embodiments, the one or more costimulatory molecules is selected from 4-1BB and CD40L. In some embodiments, the costimulatory molecule is OX40.

The impact of the GAL9 antigen binding molecule on surface expression of the one or more costimulatory molecules may be determined by any suitable means. For instance, the impact of the GAL9 antigen binding molecule on surface expression of the one or more costimulatory molecules may be determined in vivo, ex vivo, or in vitro.

In some embodiments, the GAL9 antigen binding molecule decreases surface expression of the one or more costimulatory molecules on activated immune cells as compared to activated immune cells treated with a control agent. Exemplary control agents are described herein. In particular embodiments, a control agent is an isotype control binding molecule that does not bind GAL9.

In some embodiments, the GAL9 antigen binding molecule decreases 4-1BB surface expression on activated CD8⁺ T-cells, relative to activated CD8⁺ T-cells treated with the control agent. In some embodiments, activated CD8⁺ T-cells treated with the GAL9 antigen binding molecule exhibits at least about a 0.1× decrease, 0.2× decrease, 0.3× decrease, 0.4× decrease, 0.5× decrease, or a 0.6× decrease in 4-1BB surface expression, relative to activated CD8⁺ T-cells treated with the control agent. In some embodiments, activated CD8⁺ T-cells treated with the GAL9 antigen binding molecule exhibits about a 0.1×-0.2× decrease, 0.2×-0.3× decrease, 0.3×-0.4× decrease, 0.4×-0.5× decrease, or a 0.5×-0.6× decrease in 4-1BB surface expression, relative to activated CD8⁺ T-cells treated with the control agent.

In some embodiments, the GAL9 antigen binding molecule decreases CD40L surface expression of activated CD8⁺ T-cells, relative to activated CD8⁺ T-cells treated with the control agent. In some embodiments, activated CD8⁺ T-cells treated with the GAL9 antigen binding molecule exhibits at least about a 0.1× decrease, 0.2× decrease, 0.3× decrease, 0.4× decrease, or a 0.5× decrease in CD40L surface expression relative to activated CD8⁺ T-cells treated with the control agent. In some embodiments, activated CD8⁺ T-cells treated with the GAL9 antigen binding molecule exhibits about a 0.1×-0.2× decrease, 0.2×-0.3× decrease, 0.3×-0.4× decrease, or a 0.4×-0.5× decrease in CD40L surface expression, relative to activated CD8⁺ T-cells treated with the control agent.

In some embodiments, the GAL9 antigen binding molecule decreases OX40 surface expression of activated CD8⁺ T-cells, relative to activated CD8⁺ T-cells treated with the control agent. In some embodiments, activated CD8⁺ T-cells treated with the GAL9 antigen binding molecule exhibits about at least a 0.1× decrease, 0.2× decrease, 0.3× decrease, 0.4× decrease, 0.5× decrease, or a 0.6× decrease in OX40 surface expression relative to activated CD8⁺ T-cells treated with the control agent. In some embodiments, activated CD8⁺ T-cells treated with the GAL9 antigen binding molecule exhibits about a 0.1×-0.2× decrease, 0.2×-0.3× decrease, 0.3×-0.4× decrease, 0.4×-0.5× decrease, or a 0.5×-0.6× decrease in OX40 surface expression, relative to activated CD8⁺ T-cells treated with the control agent.

The disclosure also provides for GAL9 antigen binding molecules that have various clinical benefits that improve the health of a subject with an autoimmune or inflammatory disease. The subject can be a mammal. The mammal can be a mouse. In some embodiments, the mammal is a human.

In some embodiments, the GAL9 antigen binding molecule reduces an autoimmune response in a subject. In some embodiments, the GAL9 antigen binding molecule reduces inflammation in the subject Inflammation can be systemic or localized in an organ or tissue. In some embodiments, the GAL9 antigen binding molecule prolongs remission of a disease or condition in a subject. In some embodiments, the GAL9 antigen binding molecule induces remission in a subject. In some embodiments, the GAL9 antigen binding molecule re-establishes immune tolerance (e.g., improved cytokine profile or environment) in a subject. Re-establishing immune tolerance can be a decrease in a proinflammatory cytokine, an increase in an inhibitory cytokine, or a combination thereof. In some embodiments, the GAL9 antigen binding molecule improves organ function in a subject. In some embodiments, the GAL9 antigen binding molecule reduces the risk/likelihood of disease progression or development of a second disease, such as cancer or an infection. In some embodiments, the GAL9 antigen binding molecule increases the overall survival of a subject.

6.4.2. Variable Regions

In typical embodiments, the GAL9 binding molecules have variable region domain amino acid sequences of an antibody, including VH and VL antibody domain sequences. VH and VL sequences are described in greater detail below in Sections 6.4.2.1 and 6.4.2.2, respectively.

6.4.2.1. VII Regions

In typical embodiments, the GAL9 binding molecules described herein comprise antibody heavy chain variable domain sequences. In a typical antibody arrangement in both nature and in the GAL9 binding molecules described herein, a specific VH amino acid sequence associates with a specific VL amino acid sequence to form an antigen-binding site. In various embodiments, VH amino acid sequences are mammalian sequences, including human sequences, synthesized sequences, or combinations of non-human mammalian, mammalian, and/or synthesized sequences, as described in further detail above in Sections 6.4.2.3 and 6.4.2.4. In various embodiments, VH amino acid sequences are mutated sequences of naturally occurring sequences.

6.4.2.2. VL Regions

The VL amino acid sequences useful in the GAL9 binding molecules described herein are antibody light chain variable domain sequences. In a typical arrangement in both natural antibodies and the antibody constructs described herein, a specific VL amino acid sequence associates with a specific VH amino acid sequence to form an antigen-binding site. In various embodiments, the VL amino acid sequences are mammalian sequences, including human sequences, synthesized sequences, or combinations of human, non-human mammalian, mammalian, and/or synthesized sequences, as described in further detail below in Sections 6.4.2.3 and 6.4.2.4.

In various embodiments, VL amino acid sequences are mutated sequences of naturally occurring sequences. In certain embodiments, the VL amino acid sequences are lambda (λ) light chain variable domain sequences. In certain embodiments, the VL amino acid sequences are kappa (κ) light chain variable domain sequences. In a preferred embodiment, the VL amino acid sequences are kappa (κ) light chain variable domain sequences.

6.4.2.3. Complementarity Determining Regions

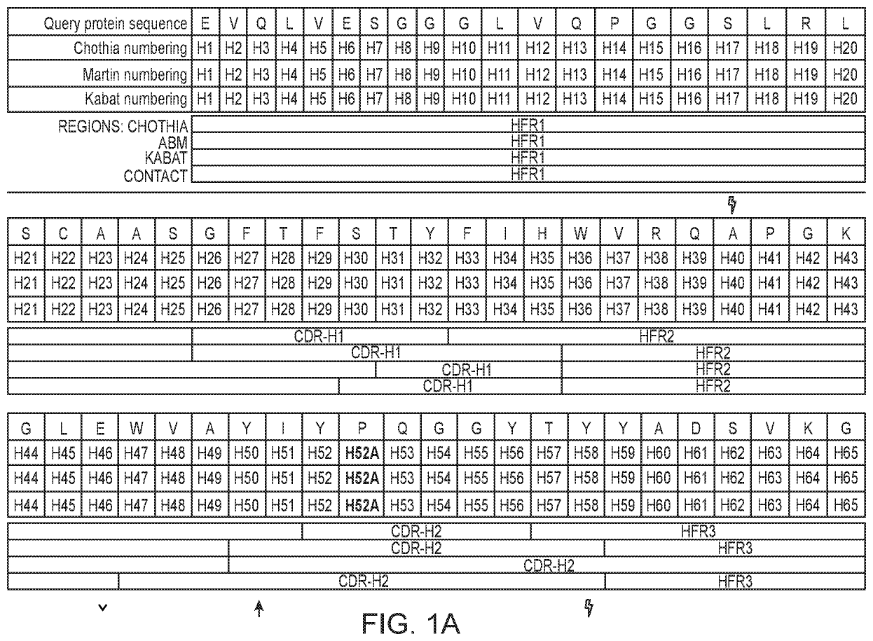

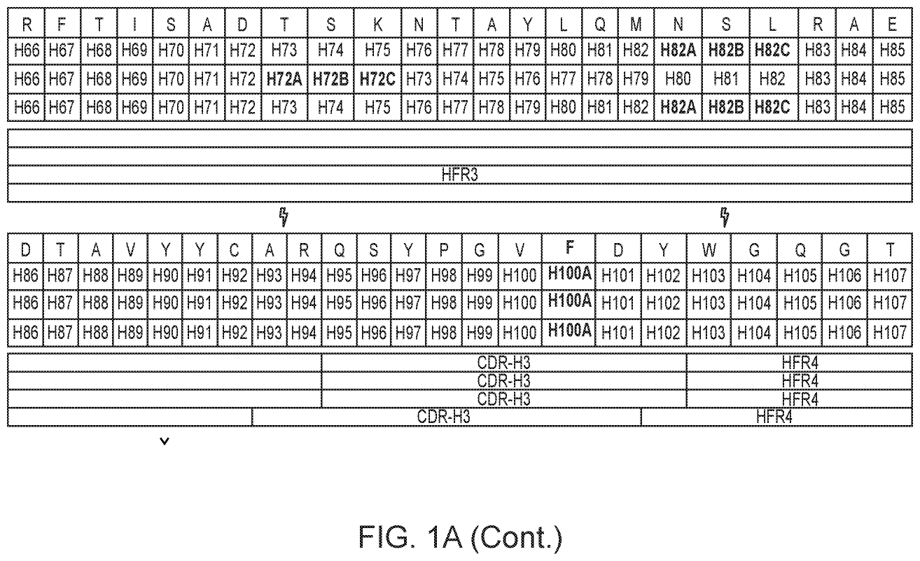

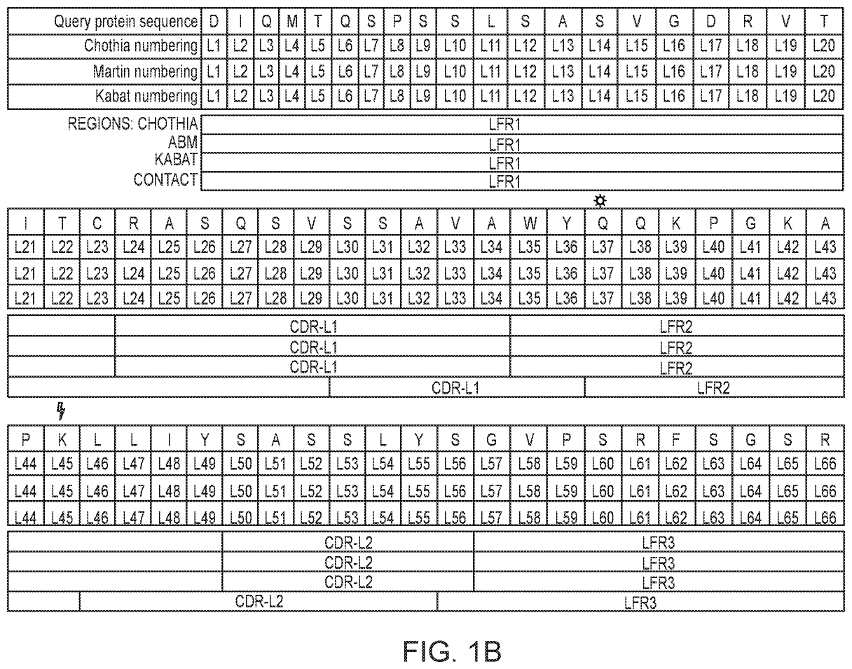

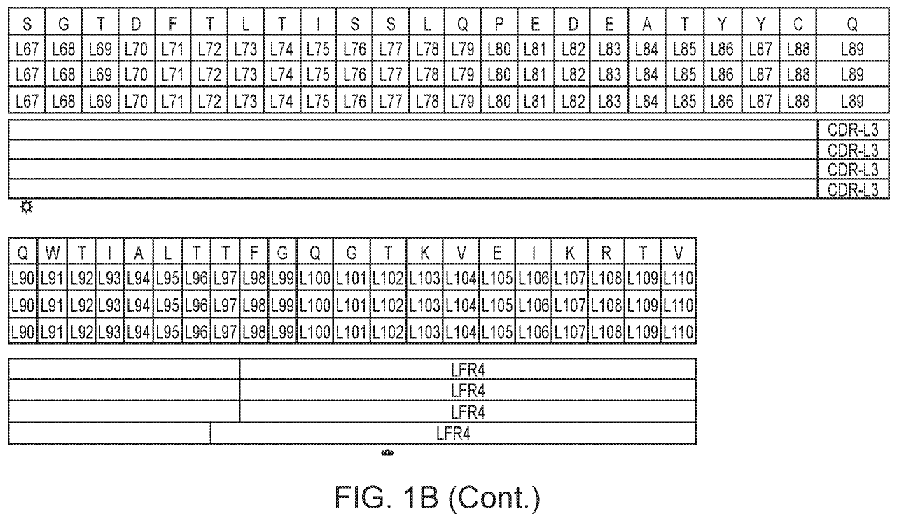

The VH and VL amino acid sequences comprise highly variable sequences termed “complementarity determining regions” (CDRs), typically three CDRs (CDR1, CDR2, and CDR3). In a variety of embodiments, the CDRs are mammalian sequences, including, but not limited to, mouse, rat, hamster, rabbit, camel, donkey, goat, and human sequences. In a preferred embodiment, the CDRs are human sequences. In various embodiments, the CDRs are naturally occurring sequences. In various embodiments, the CDRs are naturally occurring sequences that have been mutated to alter the binding affinity of the antigen-binding site for a particular antigen or epitope. In certain embodiments, the naturally occurring CDRs have been mutated in an in vivo host through affinity maturation and somatic hypermutation. In certain embodiments, the CDRs have been mutated in vitro through methods including, but not limited to, PCR-mutagenesis and chemical mutagenesis. In various embodiments, the CDRs are synthesized sequences including, but not limited to, CDRs obtained from random sequence CDR libraries and rationally designed CDR libraries. Martin numbering scheme was used to determine the CDR boundaries. See FIGS. 1A-1B as applied to the P9-01 anti-human GALS candidate provided herein.

In various embodiments, CDRs identified as binding an antigen of interest are further mutated (i.e., “affinity matured”) to achieve a desired binding characteristic, such as an increased affinity for the antigen of interest relative to the original CDR. For example, targeted introduction of diversity into the CDRs, including those CDRs identified to bind an antigen of interest, can be introduced using degenerate oligonucleotides. Various randomization schemes can be employed. For example, “soft-randomization” can be used that provides a high bias towards the identity of wild-type sequence at a given amino acid position, such as allowing a given position in CDRs to vary among all twenty amino acids while biasing towards the wild-type sequence by doping the four bases at each codon position at non-equivalent level. As an illustrative example of soft-randomization, if achieving approximately 50% of the wild-type sequence is desired, each base of each codon is kept 70% wild-type and 10% each of other nucleotides and the degenerate oligonucleotides are used to make a focused phage library around the selected CDRs with the resulting phage particles used for phage panning under various stringent selection conditions depending on the need.

6.4.2.4. Framework Regions and CDR Grafting

The VH and VL amino acid sequences comprise “framework region” (FR) sequences. FRs are generally conserved sequence regions that act as a scaffold for interspersed CDRs (see Section 6.4.2.3), typically in a FR1-CDR1-FR2-CDR2-FR3-CDR3-FR4 arrangement (from N-terminus to C-terminus). In a variety of embodiments, the FRs are mammalian sequences, including, but not limited to mouse, rat, hamster, rabbit, camel, donkey, goat, and human sequences. In a preferred embodiment, the FRs are human sequences. In various embodiments, the FRs are naturally occurring sequences. In various embodiments, the FRs are synthesized sequences including, but not limited, rationally designed sequences.

In a variety of embodiments, the FRs and the CDRs are both from the same naturally occurring variable domain sequence. In a variety of embodiments, the FRs and the CDRs are from different variable domain sequences, wherein the CDRs are grafted onto the FR scaffold with the CDRs providing specificity for a particular antigen. In certain embodiments, the grafted CDRs are all derived from the same naturally occurring variable domain sequence. In certain embodiments, the grafted CDRs are derived from different variable domain sequences. In certain embodiments, the grafted CDRs are synthesized sequences including, but not limited to, CDRs obtained from random sequence CDR libraries and rationally designed CDR libraries. In certain embodiments, the grafted CDRs and the FRs are from the same species. In certain embodiments, the grafted CDRs and the FRs are from different species. In a preferred grafted CDR embodiment, an antibody is “humanized”, wherein the grafted CDRs are non-human mammalian sequences including, but not limited to, mouse, rat, hamster, rabbit, camel, donkey, and goat sequences, and the FRs are human sequences. Humanized antibodies are discussed in more detail in U.S. Pat. No. 6,407,213, the entirety of which is hereby incorporated by reference for all it teaches. In various embodiments, portions or specific sequences of FRs from one species are used to replace portions or specific sequences of another species' FRs.

6.4.3. Exemplary Amino Acid Sequences of the GAL9 Binding Molecules

In various embodiments, the GAL9 binding molecule comprises a particular VH CDR3 (CDR-H3) sequence and a particular VL CDR3 (CDR-L3) sequence.

In some embodiments, the GAL9 binding molecule comprises the CDR-H3 and the CDR-L3 from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57. VH CDR amino acid sequences of the ABS clones are disclosed in Table 3. VL CDR amino acid sequences of the ABS clones are disclosed in Table 4. For clarity, each GAL9 ABS clone is assigned a unique ABS clone number which is used throughout this disclosure.

In one currently preferred embodiment, the GAL9 binding molecule comprises the CDR-H3 and CDR-L3 of ABS clone P9-11.

In some embodiments, the GAL9 binding molecule comprises all three VH CDRs from one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57. In one currently preferred embodiment, the GAL9 binding molecule comprises all three VH CDRs from ABS clone P9-11.

In some embodiments, the GAL9 binding molecule comprises all three VL CDRs from one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57. In one currently preferred embodiment, the GAL9 binding molecule comprises all three VL CDRs from ABS clone P9-11.

In some embodiments, the GAL9 binding molecule comprises all six CDRs from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57. In one currently preferred embodiment, the GAL9 binding molecule comprises all six CDRs from ABS clone P9-11.

In some embodiments, the GAL9 binding molecule comprises a VH amino acid sequence, a VL amino acid sequence, or a VH and VL amino acid sequence from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57. Full immunoglobulin heavy chain and immunoglobulin light chain sequences, as well as VH and VL amino acid sequences, are provided in Table 6. In one currently preferred embodiment, the GAL9 binding molecule comprises a VH amino acid sequence, a VL amino acid sequence, or a VH and VL amino acid sequence from ABS clone P9-11.

In some embodiments, the GAL9 binding molecule comprises the full IgG heavy chain sequence and the full IgG light chain sequence from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57. In one currently preferred embodiment, the GAL9 binding molecule comprises the full IgG heavy chain sequence and the full IgG light chain sequence from ABS clone P9-11.

6.4.4. Constant Regions

In some embodiments, the GAL9 binding molecules comprise an antibody constant region domain sequence. Constant region domain amino acid sequences, as described herein, are sequences of a constant region domain of an antibody. Constant regions can refer to CH1, CH2, CH3, CH4, or CL constant domain.

In a variety of embodiments, the constant region sequences are mammalian sequences, including, but not limited to, mouse, rat, hamster, rabbit, camel, donkey, goat, and human sequences. In a preferred embodiment, the constant region sequences are human sequences. In certain embodiments, the constant region sequences are from an antibody light chain. In particular embodiments, the constant region sequences are from a lambda or kappa light chain. In certain embodiments, the constant region sequences are from an antibody heavy chain. In particular embodiments, the constant region sequences are an antibody heavy chain sequence that is an IgA1, IgA2, IgD, IgE, IgG1, IgG2, IgG3, IgG4, or IgM isotype. In a specific embodiment, the constant region sequences are from an IgG isotype. In a preferred embodiment, the constant region sequences are from an IgG1 isotype.

Exemplary constant regions and modifications thereof are described in WO2018075692, which is hereby incorporated by reference in its entirety.

6.4.4.1. CH1 and CL Regions

CH1 amino acid sequences, as described herein, are sequences of the second domain of an antibody heavy chain, with reference from the N-terminus to C-terminus of a native antibody heavy chain architecture. In certain embodiments, the CH1 sequences are endogenous sequences. In a variety of embodiments, the CH1 sequences are mammalian sequences, including, but not limited to mouse, rat, hamster, rabbit, camel, donkey, goat, and human sequences. In a preferred embodiment, the CH1 sequences are human sequences. In certain embodiments, the CH1 sequences are from an IgA1, IgA2, IgD, IgE, IgG1, IgG2, IgG3, IgG4, or IgM isotype. In a preferred embodiment, the CH1 sequences are from an IgG1 isotype. In preferred embodiments, the CH1 sequence is UniProt accession number P01857 amino acids 1-98.

The CL amino acid sequences useful in the GALS binding molecules described herein are antibody light chain constant domain sequences, with reference to a native antibody light chain architecture. In certain embodiments, the CL sequences are endogenous sequences. In a variety of embodiments, the CL sequences are mammalian sequences, including, but not limited to mouse, rat, hamster, rabbit, camel, donkey, goat, and human sequences. In a preferred embodiment, CL sequences are human sequences.

In certain embodiments, the CL amino acid sequences are lambda (λ) light chain constant domain sequences. In particular embodiments, the CL amino acid sequences are human lambda light chain constant domain sequences. In preferred embodiments, the lambda (λ) light chain sequence is UniProt accession number P0CG04.

In certain embodiments, the CL amino acid sequences are kappa (κ) light chain constant domain sequences. In a preferred embodiment, the CL amino acid sequences are human kappa (κ) light chain constant domain sequences. In a preferred embodiment, the kappa light chain sequence is UniProt accession number P01834.

In certain embodiments, the CH1 sequence and the CL sequences are both endogenous sequences. In certain embodiments, the CH1 sequence and the CL sequences separately comprise respectively orthogonal modifications in endogenous CH1 and CL sequences, as discussed below in greater detail in Section 6.4.4.1. CH1 and CL sequences can also be portions thereof, either of an endogenous or modified sequence, such that a domain having the CH1 sequence, or portion thereof, can associate with a domain having the CL sequence, or portion thereof.

6.4.4.2. CH1 and CL Orthogonal Modifications

In certain embodiments, the CH1 sequence and the CL sequences separately comprise respectively orthogonal modifications in endogenous CH1 and CL sequences. Orthogonal mutations, in general, are described in more detail below in Sections 6.4.6.1-6.4.6.3.

In particular embodiments, the orthogonal modifications in endogenous CH1 and CL sequences are an engineered disulfide bridge selected from engineered cysteines at position 138 of the CH1 sequence and position 116 of the CL sequence, at position 128 of the CH1 sequence and position 119 of the CL sequence, or at position 129 of the CH1 sequence and position 210 of the CL sequence, as numbered and discussed in more detail in U.S. Pat. Nos. 8,053,562 and 9,527,927, each incorporated herein by reference in its entirety. In a preferred embodiment, the engineered cysteines are at position 128 of the CH1 sequence and position 118 of the CL Kappa sequence, as numbered by the Eu index.

In a series of preferred embodiments, the mutations that provide non-endogenous cysteine amino acids are a F118C mutation in the CL sequence with a corresponding A141C in the CH1 sequence, or a F118C mutation in the CL sequence with a corresponding L128C in the CH1 sequence, or a S162C mutations in the CL sequence with a corresponding P171C mutation in the CH1 sequence, as numbered by the Eu index.

In a variety of embodiments, the orthogonal mutations in the CL sequence and the CH1 sequence are charge-pair mutations. In specific embodiments the charge-pair mutations are a F118S, F118A or F118V mutation in the CL sequence with a corresponding A141L in the CH1 sequence, or a T129R mutation in the CL sequence with a corresponding K147D in the CH1 sequence, as numbered by the Eu index and described in greater detail in Bonisch et al. (Protein Engineering, Design & Selection, 2017, pp. 1-12), herein incorporated by reference for all that it teaches. In a series of preferred embodiments, the charge-pair mutations are a N138K mutation in the CL sequence with a corresponding G166D in the CH1 sequence, or a N138D mutation in the CL sequence with a corresponding G166K in the CH1 sequence, as numbered by the Eu index.

6.4.4.3. CH2 Regions

In the GAL9 binding molecules described herein, the GAL9 binding molecules can have a CH2 amino acid sequence. CH2 amino acid sequences, as described herein, are CH2 amino acid sequences of the third domain of an antibody heavy chain, with reference from the N-terminus to C-terminus of a native antibody heavy chain architecture. In a variety of embodiments, the CH2 sequences are mammalian sequences, including but not limited to mouse, rat, hamster, rabbit, camel, donkey, goat, and human sequences. In a preferred embodiment, the CH2 sequences are human sequences. In certain embodiments, the CH2 sequences are from an IgA1, IgA2, IgD, IgE, IgG1, IgG2, IgG3, IgG4, or IgM isotype. In a preferred embodiment, the CH2 sequences are from an IgG1 isotype.

In certain embodiments, the CH2 sequences are endogenous sequences. In particular embodiments, the sequence is UniProt accession number P01857 amino acids 111-223.

In a series of embodiments, a GAL9 binding molecule has more than one paired set of CH2 domains that have CH2 sequences, wherein a first set has CH2 amino acid sequences from a first isotype and one or more orthologous sets of CH2 amino acid sequences from another isotype. The orthologous CH2 amino acid sequences, as described herein, are able to interact with CH2 amino acid sequences from a shared isotype, but not significantly interact with the CH2 amino acid sequences from another isotype present in the GAL9 binding molecule. In particular embodiments, all sets of CH2 amino acid sequences are from the same species. In preferred embodiments, all sets of CH2 amino acid sequences are human CH2 amino acid sequences. In other embodiments, the sets of CH2 amino acid sequences are from different species. In particular embodiments, the first set of CH2 amino acid sequences is from the same isotype as the other non-CH2 domains in the GAL9 binding molecule. In a specific embodiment, the first set has CH2 amino acid sequences from an IgG isotype and the one or more orthologous sets have CH2 amino acid sequences from an IgM or IgE isotype. In certain embodiments, one or more of the sets of CH2 amino acid sequences are endogenous CH2 sequences. In other embodiments, one or more of the sets of CH2 amino acid sequences are endogenous CH2 sequences that have one or more mutations. In particular embodiments, the one or more mutations are orthogonal knob-hole mutations, orthogonal charge-pair mutations, or orthogonal hydrophobic mutations. Orthologous CH2 amino acid sequences useful for the GAL9 binding molecules are described in more detail in international PCT applications WO2017/011342 and WO2017/106462, herein incorporated by reference in their entirety.

6.4.4.4. CH3 Regions

CH3 amino acid sequences, as described herein, are sequences of the C-terminal domain of an antibody heavy chain, with reference from the N-terminus to C-terminus of a native antibody heavy chain architecture.

In a variety of embodiments, the CH3 sequences are mammalian sequences, including, but not limited to, mouse, rat, hamster, rabbit, camel, donkey, goat, and human sequences. In a preferred embodiment, the CH3 sequences are human sequences. In certain embodiments, the CH3 sequences are from an IgA1, IgA2, IgD, IgE, IgM, IgG1, IgG2, IgG3, IgG4 isotype or CH4 sequences from an IgE or IgM isotype. In a specific embodiment, the CH3 sequences are from an IgG isotype. In a preferred embodiment, the CH3 sequences are from an IgG1 isotype.

In certain embodiments, the CH3 sequences are endogenous sequences. In particular embodiments, the CH3 sequence is UniProt accession number P01857 amino acids 224-330. In various embodiments, a CH3 sequence is a segment of an endogenous CH3 sequence. In particular embodiments, a CH3 sequence has an endogenous CH3 sequence that lacks the N-terminal amino acids G224 and Q225. In particular embodiments, a CH3 sequence has an endogenous CH3 sequence that lacks the C-terminal amino acids P328, G329, and K330. In particular embodiments, a CH3 sequence has an endogenous CH3 sequence that lacks both the N-terminal amino acids G224 and Q225 and the C-terminal amino acids P328, G329, and K330. In preferred embodiments, a GALS binding molecule has multiple domains that have CH3 sequences, wherein a CH3 sequence can refer to both a full endogenous CH3 sequence as well as a CH3 sequence that lacks N-terminal amino acids, C-terminal amino acids, or both.

In certain embodiments, the CH3 sequences are endogenous sequences that have one or more mutations. In particular embodiments, the mutations are one or more orthogonal mutations that are introduced into an endogenous CH3 sequence to guide specific pairing of specific CH3 sequences, as described in more detail below in Sections 6.4.6.1-6.4.6.3.

In certain embodiments, the CH3 sequences are engineered to reduce immunogenicity of the antibody by replacing specific amino acids of one allotype with those of another allotype and referred to herein as isoallotype mutations, as described in more detail in Stickler et al. (Genes Immun. 2011 April; 12(3): 213-221), which is herein incorporated by reference for all that it teaches. In particular embodiments, specific amino acids of the Glml allotype are replaced. In a preferred embodiment, isoallotype mutations D356E and L358M are made in the CH3 sequence.

In some embodiments, an IgG1 CH3 amino acid sequence comprises the following mutational changes: P343V; Y349C; and a tripeptide insertion, 445P, 446G, 447K. In other preferred embodiments, domain B has a human IgG1 CH3 sequence with the following mutational changes: T366K; and a tripeptide insertion, 445K, 446S, 447C. In still other preferred embodiments, domain B has a human IgG1 CH3 sequence with the following mutational changes: Y349C and a tripeptide insertion, 445P, 446G, 447K.

In some embodiments, an IgG1 CH3 amino acid sequence comprises a 447C mutation incorporated into an otherwise endogenous CH3 sequence.

6.4.5. Antigen Binding Sites

In some embodiments, a VL or VH amino acid sequence and a cognate VL or VH amino acid sequence are associated and form a first antigen binding site (ABS). The antigen binding site (ABS) is capable of specifically binding an epitope of an antigen. Antigen binding by an ABS is described in greater detail below in Section 6.4.5.1.

In alternative embodiments, e.g., wherein the GAL9 binding molecule is a single domain antibody, a VH or VL amino acid sequence forms the first ABS.

In some embodiments, the GAL9 antigen binding molecule comprises a second ABS. In some embodiments, the second ABS is specific for the same GAL9 antigen as the first ABS. In some embodiments, the second ABS specifically binds the same epitope of the same GAL9 antigen as the first ABS. In some embodiments, the second ABS is identical to the first ABS.

In some embodiments, the second ABS is specific for a different epitope of the first GAL9 antigen. For example if the first ABS comprises CDRs or variable domains from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57. The second ABS may comprise CDRs or variable domains from another ABS clone selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

In some embodiments, the GAL9 antigen binding molecule is multispecific, e.g., the second ABS of the GAL9 antigen binding molecule specifically binds an antigen that is different than the GAL9 antigen specifically bound by the first ABS.

6.4.5.1. Binding of Antigen by ABS

An ABS, and the GAL9 binding molecule comprising such ABS, is said to “recognize” the epitope (or more generally, the antigen) to which the ABS specifically binds, and the epitope (or more generally, the antigen) is said to be the “recognition specificity” or “binding specificity” of the ABS.

The ABS is said to bind to its specific antigen or epitope with a particular affinity. As described herein, “affinity” refers to the strength of interaction of non-covalent intermolecular forces between one molecule and another. The affinity, i.e. the strength of the interaction, can be expressed as a dissociation equilibrium constant (K_{D}), wherein a lower K_{D} value refers to a stronger interaction between molecules. K_{D} values of antibody constructs are measured by methods well known in the art including, but not limited to, bio-layer interferometry (e.g., Octet/FORTEBIO®), surface plasmon resonance (SPR) technology (e.g., Biacore®), and cell binding assays. For purposes herein, affinities are dissociation equilibrium constants measured by bio-layer interferometry using Octet/FORTEBIO®.

“Specific binding,” as used herein, refers to an affinity between an ABS and its cognate antigen or epitope in which the K_{D} value is below 10⁻⁶M, 10⁻⁷M, 10⁻⁸M, 10⁻⁹M, or 10⁻¹° M.

The number of ABSs in a GAL9 binding molecule as described herein defines the “valency” of the GAL9 binding molecule. A GAL9 binding molecule having a single ABS is “monovalent”. A GAL9 binding molecule having a plurality of ABSs is said to be “multivalent”. A multivalent GAL9 binding molecule having two ABSs is “bivalent.” A multivalent GAL9 binding molecule having three ABSs is “trivalent.” A multivalent GAL9 binding molecule having four ABSs is “tetravalent.”

In various multivalent embodiments, all of the plurality of ABSs have the same recognition specificity. Such a GAL9 binding molecule is a “monospecific” “multivalent” binding construct. In other multivalent embodiments, at least two of the plurality of ABSs have different recognition specificities. Such GAL9 binding molecules are multivalent and “multispecific”. In multivalent embodiments in which the ABSs collectively have two recognition specificities, the GAL9 binding molecule is “bispecific.” In multivalent embodiments in which the ABSs collectively have three recognition specificities, the GAL9 binding molecule is “trispecific.”

In multivalent embodiments in which the ABSs collectively have a plurality of recognition specificities for different epitopes present on the same antigen, the GAL9 binding molecule is “multiparatopic.” Multivalent embodiments in which the ABSs collectively recognize two epitopes on the same antigen are “biparatopic.”

In various multivalent embodiments, multivalency of the GAL9 binding molecule improves the avidity of the GAL9 binding molecule for a specific target. As described herein, “avidity” refers to the overall strength of interaction between two or more molecules, e.g., a multivalent GAL9 binding molecule for a specific target, wherein the avidity is the cumulative strength of interaction provided by the affinities of multiple ABSs. Avidity can be measured by the same methods as those used to determine affinity, as described above. In certain embodiments, the avidity of a GAL9 binding molecule for a specific target is such that the interaction is a specific binding interaction, wherein the avidity between two molecules has a K_{D} value below 10⁻⁶M, 10⁻⁷M, 10⁻⁸M, 10⁻⁹M, or 10⁻¹⁰M. In certain embodiments, the avidity of a GAL9 binding molecule for a specific target has a K_{D} value such that the interaction is a specific binding interaction, wherein the one or more affinities of individual ABSs do not have has a K_{D} value that qualifies as specifically binding their respective antigens or epitopes on their own. In certain embodiments, the avidity is the cumulative strength of interaction provided by the affinities of multiple ABSs for separate antigens on a shared specific target or complex, such as separate antigens found on an individual cell. In certain embodiments, the avidity is the cumulative strength of interaction provided by the affinities of multiple ABSs for separate epitopes on a shared individual antigen.

6.4.6. Orthogonal Modifications

In the GAL9 binding molecules described herein, a GAL9 binding molecule can have constant region domains comprising orthogonal modifications. Constant region domain amino acid sequences are described in greater detail above in Section 6.4.4.

“Orthogonal modifications” or synonymously “orthogonal mutations” as described herein are one or more engineered mutations in an amino acid sequence of an antibody domain that increase the affinity of binding of a first domain having orthogonal modification for a second domain having a complementary orthogonal modification. In certain embodiments, the orthogonal modifications decrease the affinity of a domain having the orthogonal modifications for a domain lacking the complementary orthogonal modifications. In certain embodiments, orthogonal modifications are mutations in an endogenous antibody domain sequence. In a variety of embodiments, orthogonal modifications are modifications of the N-terminus or C-terminus of an endogenous antibody domain sequence including, but not limited to, amino acid additions or deletions. In particular embodiments, orthogonal modifications include, but are not limited to, engineered disulfide bridges, knob-in-hole mutations, and charge-pair mutations, as described in greater detail below in Sections 6.4.6.1-6.4.6.3. In particular embodiments, orthogonal modifications include a combination of orthogonal modifications selected from, but not limited to, engineered disulfide bridges, knob-in-hole mutations, and charge-pair mutations. In particular embodiments, the orthogonal modifications can be combined with amino acid substitutions that reduce immunogenicity, such as isoallotype mutations, as described in greater detail above in Section 6.4.4.4.

6.4.6.1. Orthogonal Engineered Disulfide Bridges

In a variety of embodiments, the orthogonal modifications comprise mutations that generate engineered disulfide bridges between a first and a second domain. As described herein, “engineered disulfide bridges” are mutations that provide non-endogenous cysteine amino acids in two or more domains such that a non-native disulfide bond forms when the two or more domains associate. Engineered disulfide bridges are described in greater detail in Merchant et al. (Nature Biotech (1998) 16:677-681), the entirety of which is hereby incorporated by reference for all it teaches. In certain embodiments, engineered disulfide bridges improve orthogonal association between specific domains. In a particular embodiment, the mutations that generate engineered disulfide bridges are a K392C mutation in one of a first or second CH3 domains, and a D399C in the other CH3 domain. In a preferred embodiment, the mutations that generate engineered disulfide bridges are a S354C mutation in one of a first or second CH3 domains, and a Y349C in the other CH3 domain. In another preferred embodiment, the mutations that generate engineered disulfide bridges are a 447C mutation in both the first and second CH3 domains that are provided by extension of the C-terminus of a CH3 domain incorporating a KSC tripeptide sequence.

6.4.6.2. Orthogonal Knob-Hole Mutations

In a variety of embodiments, orthogonal modifications comprise knob-hole (synonymously, knob-in-hole) mutations. As described herein, knob-hole mutations are mutations that change the steric features of a first domain's surface such that the first domain will preferentially associate with a second domain having complementary steric mutations relative to association with domains without the complementary steric mutations. Knob-hole mutations are described in greater detail in U.S. Pat. Nos. 5,821,333 and 8,216,805, each of which is incorporated herein in its entirety. In various embodiments, knob-hole mutations are combined with engineered disulfide bridges, as described in greater detail in Merchant et al. (Nature Biotech (1998) 16:677-681)), incorporated herein by reference in its entirety. In various embodiments, knob-hole mutations, isoallotype mutations, and engineered disulfide mutations are combined.

In certain embodiments, the knob-in-hole mutations are a T366Y mutation in a first domain, and a Y407T mutation in a second domain. In certain embodiments, the knob-in-hole mutations are a F405A in a first domain, and a T394W in a second domain. In certain embodiments, the knob-in-hole mutations are a T366Y mutation and a F405A in a first domain, and a T394W and a Y407T in a second domain. In certain embodiments, the knob-in-hole mutations are a T366W mutation in a first domain, and a Y407A in a second domain. In certain embodiments, the combined knob-in-hole mutations and engineered disulfide mutations are a S354C and T366W mutations in a first domain, and a Y349C, a T366S, a L368A, and a Y407V mutation in a second domain. In a preferred embodiment, the combined knob-in-hole mutations, isoallotype mutations, and engineered disulfide mutations are a S354C and T366W mutations in a first domain, and a Y349C, D356E, L358M, T366S, L368A, and a Y407V mutation in a second domain.

6.4.6.3. Orthogonal Charge-pair Mutations

In a variety of embodiments, orthogonal modifications are charge-pair mutations. As used herein, charge-pair mutations are mutations that affect the charge of an amino acid in a domain's surface such that the domain will preferentially associate with a second domain having complementary charge-pair mutations relative to association with domains without the complementary charge-pair mutations. In certain embodiments, charge-pair mutations improve orthogonal association between specific domains. Charge-pair mutations are described in greater detail in U.S. Pat. Nos. 8,592,562, 9,248,182, and 9,358,286, each of which is incorporated by reference herein for all they teach. In certain embodiments, charge-pair mutations improve stability between specific domains. In a preferred embodiment, the charge-pair mutations are a T366K mutation in a first domain, and a L351D mutation in the other domain.

In specific embodiments, the orthogonal mutations are charge-pair mutations at the VH/VL interface. In preferred embodiments, the charge-pair mutations at the VH/VL interface are a Q39E in VH with a corresponding Q38K in VL, or a Q39K in VH with a corresponding Q38E in VL, as described in greater detail in Igawa et al. (Protein Eng. Des. Sel., 2010, vol. 23, 667-677), herein incorporated by reference for all it teaches.

6.4.7. Trivalent and Tetravalent GAL9 binding molecules

In another series of embodiments, the GAL9 binding molecules have three antigen binding sites and are therefore termed “trivalent.” In a variety of embodiments, the GAL9 binding molecules have 4 antigen binding sites and are therefore termed “tetravalent.”

6.5. GAL9 binding molecule architecture

The antigen binding sites described herein, including specific CDR subsets, can be formatted into any binding molecule architecture including, but not limited to, full-length antibodies, Fab fragments, Fvs, scFvs, tandem scFvs, Diabodies, scDiabodies, DARTs, tandAbs, minibodies, camelid VHH, and other antibody fragments or formats known to those skilled in the art. Exemplary antibody and antibody fragment formats are described in detail in Brinkmann et al. (MABS, 2017, Vol. 9, No. 2, 182-212), herein incorporated by reference for all that it teaches. The antigen binding sites described herein, including specific CDR subsets, can also be formatted into a “B-body” format, as described in more detail in US pre-grant publication no. US 2018/0118811 and International Application Pub. No. WO 2018/075692, each of which is herein incorporated by reference in their entireties.

6.6. Further modifications

In a further series of embodiments, the GAL9 binding molecule has additional modifications.

6.6.1. Antibody-Drug Conjugates

In various embodiments, the GAL9 binding molecule is conjugated to a therapeutic agent (i.e. drug) to form a GAL9 binding molecule-drug conjugate. Therapeutic agents include, but are not limited to, chemotherapeutic agents, imaging agents (e.g. radioisotopes), immune modulators (e.g. cytokines, chemokines, or checkpoint inhibitors), and toxins (e.g. cytotoxic agents). In certain embodiments, the therapeutic agents are attached to the GAL9 binding molecule through a linker peptide, as discussed in more detail below in Section 6.6.3.

Methods of preparing antibody-drug conjugates (ADCs) that can be adapted to conjugate drugs to the GAL9 binding molecules disclosed herein are described, e.g., in U.S. Pat. No. 8,624,003 (pot method), U.S. Pat. No. 8,163,888 (one-step), U.S. Pat. No. 5,208,020 (two-step method), U.S. Pat. Nos. 8,337,856, 5,773,001, 7,829,531, 5,208,020, 7,745,394, WO 2017/136623, WO 2017/015502, WO 2017/015496, WO 2017/015495, WO 2004/010957, WO 2005/077090, WO 2005/082023, WO 2006/065533, WO 2007/030642, WO 2007/103288, WO 2013/173337, WO 2015/057699, WO 2015/095755, WO 2015/123679, WO 2015/157286, WO 2017/165851, WO 2009/073445, WO 2010/068759, WO 2010/138719, WO 2012/171020, WO 2014/008375, WO 2014/093394, WO 2014/093640, WO 2014/160360, WO 2015/054659, WO 2015/195925, WO 2017/160754, Storz (MAbs. 2015 November-December; 7(6): 989-1009), Lambert et al. (Adv Ther, 2017 34: 1015), Diamantis et al. (British Journal of Cancer, 2016, 114, 362-367), Carrico et al. (Nat Chem Biol, 2007. 3: 321-2), We et al. (Proc Natl Acad Sci USA, 2009. 106: 3000-5), Rabuka et al. (Curr Opin Chem Biol., 2011 14: 790-6), Hudak et al. (Angew Chem Int Ed Engl., 2012: 4161-5), Rabuka et al. (Nat Protoc., 2012 7:1052-67), Agarwal et al. (Proc Natl Acad Sci USA., 2013, 110: 46-51), Agarwal et al. (Bioconjugate Chem., 2013, 24: 846-851), Barfield et al. (Drug Dev. and D., 2014, 14:34-41), Drake et al. (Bioconjugate Chem., 2014, 25:1331-41), Liang et al. (J Am Chem Soc., 2014, 136:10850-3), Drake et al. (Curr Opin Chem Biol., 2015, 28:174-80), and York et al. (BMC Biotechnology, 2016, 16(1):23), each of which is hereby incorporated by reference in its entirety for all that it teaches.

6.6.2. Additional Binding Moieties

In various embodiments, the GAL9 binding molecule has modifications that comprise one or more additional binding moieties. In certain embodiments the binding moieties are antibody fragments or antibody formats including, but not limited to, full-length antibodies, Fab fragments, Fvs, scFvs, tandem scFvs, Diabodies, scDiabodies, DARTs, tandAbs, minibodies, camelid VHH, and other antibody fragments or formats known to those skilled in the art. Exemplary antibody and antibody fragment formats are described in detail in Brinkmann et al. (MABS, 2017, Vol. 9, No. 2, 182-212), herein incorporated by reference for all that it teaches.

In particular embodiments, the one or more additional binding moieties are attached to the C-terminus of the first or third polypeptide chain. In particular embodiments, the one or more additional binding moieties are attached to the C-terminus of both the first and third polypeptide chain. In particular embodiments, the one or more additional binding moieties are attached to the C-terminus of both the first and third polypeptide chains. In certain embodiments, individual portions of the one or more additional binding moieties are separately attached to the C-terminus of the first and third polypeptide chains such that the portions form the functional binding moiety.

In particular embodiments, the one or more additional binding moieties are attached to the N-terminus of any of the polypeptide chains (e.g. the first, second, third, fourth, fifth, or sixth polypeptide chains). In certain embodiments, individual portions of the additional binding moieties are separately attached to the N-terminus of different polypeptide chains such that the portions form the functional binding moiety.

In certain embodiments, the one or more additional binding moieties are specific for a different antigen or epitope of the ABSs within the GAL9 binding molecule. In certain embodiments, the one or more additional binding moieties are specific for the same antigen or epitope of the ABSs within the GAL9 binding molecule. In certain embodiments, wherein the modification is two or more additional binding moieties, the additional binding moieties are specific for the same antigen or epitope. In certain embodiments, wherein the modification is two or more additional binding moieties, the additional binding moieties are specific for different antigens or epitopes.

In certain embodiments, the one or more additional binding moieties are attached to the GAL9 binding molecule using in vitro methods including, but not limited to, reactive chemistry and affinity tagging systems, as discussed in more detail below in Section 6.6.3. In certain embodiments, the one or more additional binding moieties are attached to the GAL9 binding molecule through Fc-mediated binding (e.g. Protein A/G). In certain embodiments, the one or more additional binding moieties are attached to the GAL9 binding molecule using recombinant DNA techniques, such as encoding the nucleotide sequence of the fusion product between the GAL9 binding molecule and the additional binding moieties on the same expression vector (e.g., plasmid).

6.6.3. Functional/Reactive Groups

In various embodiments, the GAL9 binding molecule has modifications that comprise functional groups or chemically reactive groups that can be used in downstream processes, such as linking to additional moieties (e.g., drug conjugates and additional binding moieties, as discussed in more detail above in Sections 6.6.1. and 6.6.2.) and downstream purification processes.

In certain embodiments, the modifications are chemically reactive groups including, but not limited to, reactive thiols (e.g. maleimide based reactive groups), reactive amines (e.g., N-hydroxysuccinimide based reactive groups), “click chemistry” groups (e.g. reactive alkyne groups), and aldehydes bearing formylglycine (FGly). In certain embodiments, the modifications are functional groups including, but not limited to, affinity peptide sequences (e.g., HA, HIS, FLAG, GST, MBP, and Strep systems etc.). In certain embodiments, the functional groups or chemically reactive groups have a cleavable peptide sequence. In particular embodiments, the cleavable peptide is cleaved by means including, but not limited to, photocleavage, chemical cleavage, protease cleavage, reducing conditions, and pH conditions. In particular embodiments, protease cleavage is carried out by intracellular proteases. In particular embodiments, protease cleavage is carried out by extracellular or membrane associated proteases. ADC therapies adopting protease cleavage are described in more detail in Choi et al. (Theranostics, 2012; 2(2): 156-178), which is hereby incorporated by reference for all it teaches.

6.6.4. Reduced Effector Function

In certain embodiments, the GAL9 binding molecule has one or more engineered mutations in an amino acid sequence of an antibody domain that reduce the effector functions naturally associated with antibody binding. Effector functions include, but are not limited to, cellular functions that result from an Fc receptor binding to an Fc portion of an antibody, such as antibody-dependent cellular cytotoxicity (ADCC, also referred to as antibody-dependent cell-mediated cytotoxicity), complement fixation (e.g. C1q binding), antibody dependent cellular-mediated phagocytosis (ADCP), and opsonization. Exemplary engineered mutations that reduce the effector functions are described in more detail in U.S. Pub. No. 2017/0137530, Armour, et al. (Eur. J. Immunol. 29(8) (1999) 2613-2624), Shields, et al. (J. Biol. Chem. 276(9) (2001) 6591-6604), and Oganesyan, et al. (Acta Cristallographica D64 (2008) 700-704), each of which are herein incorporated by reference in its entirety.

6.7. Methods of Purification

Methods of purifying a GAL9 binding molecule are provided herein. Purification steps include, but are not limited to, purifying the GAL9 binding molecules based on protein characteristics, such as size (e.g., size exclusion chromatography), charge (e.g., ion exchange chromatography), or hydrophobicity (e.g., hydrophobicity interaction chromatography). In one embodiment, cation exchange chromatograph is performed. Other purification methods known to those skilled in the art can be performed including, but not limited to, use of Protein A, Protein G, or Protein A/G reagents. Multiple iterations of a single purification method can be performed. A combination of purification methods can be performed.

6.7.1. Assembly and Purity of Complexes

In the embodiments of the present invention, at least four distinct polypeptide chains associate together to form a complete complex, i.e., the GAL9 binding molecule. However, incomplete complexes can also form that do not contain the at least four distinct polypeptide chains. For example, incomplete complexes may form that only have one, two, or three of the polypeptide chains. In other examples, an incomplete complex may contain more than three polypeptide chains, but does not contain the at least four distinct polypeptide chains, e.g., the incomplete complex inappropriately associates with more than one copy of a distinct polypeptide chain. The method of the invention purifies the complex, i.e., the completely assembled GAL9 binding molecule, from incomplete complexes.

Methods to assess the efficacy and efficiency of the purification steps are well known to those skilled in the art and include, but are not limited to, SDS-PAGE analysis, ion exchange chromatography, size exclusion chromatography, and mass spectrometry. Purity can also be assessed according to a variety of criteria. Examples of criterion include, but are not limited to: 1) assessing the percentage of the total protein in an eluate that is provided by the completely assembled GAL9 binding molecule, 2) assessing the fold enrichment or percent increase of the method for purifying the desired products, e.g., comparing the total protein provided by the completely assembled GAL9 binding molecule in the eluate to that in a starting sample, 3) assessing the percentage of the total protein or the percent decrease of undesired products, e.g., the incomplete complexes described above, including determining the percent or the percent decrease of specific undesired products (e.g., unassociated single polypeptide chains, dimers of any combination of the polypeptide chains, or trimers of any combination of the polypeptide chains). Purity can be assessed after any combination of methods described herein.

6.8. Methods of Manufacturing

The GAL9 binding molecules described herein can readily be manufactured by expression using standard cell free translation, transient transfection, and stable transfection approaches currently used for antibody manufacture. In specific embodiments, Expi293 cells (ThermoFisher) can be used for production of the GAL9 binding molecules using protocols and reagents from ThermoFisher, such as ExpiFectamine, or other reagents known to those skilled in the art, such as polyethylenimine as described in detail in Fang et al. (Biological Procedures Online, 2017, 19:11), herein incorporated by reference for all it teaches.

The expressed proteins can be readily separated from undesired proteins and protein complexes using various purification strategies including, but not limited to, use of Protein A, Protein G, or Protein A/G reagents. Further purification can be affected using ion exchange chromatography as is routinely used in the art.

6.9. Pharmaceutical Compositions

In another aspect, pharmaceutical compositions are provided that comprise a GAL9 binding molecule as described herein and a pharmaceutically acceptable carrier or diluent. In typical embodiments, the pharmaceutical composition is sterile.

In various embodiments, the pharmaceutical composition comprises the GAL9 binding molecule at a concentration of 0.1 mg/ml-100 mg/ml. In specific embodiments, the pharmaceutical composition comprises the GAL9 binding molecule at a concentration of 0.5 mg/ml, 1 mg/ml, 1.5 mg/ml, 2 mg/ml, 2.5 mg/ml, 5 mg/ml, 7.5 mg/ml, or 10 mg/ml. In some embodiments, the pharmaceutical composition comprises the GAL9 binding molecule at a concentration of more than 10 mg/ml. In certain embodiments, the GAL9 binding molecule is present at a concentration of 20 mg/ml, 25 mg/ml, 30 mg/ml, 35 mg/ml, 40 mg/ml, 45 mg/ml, or even 50 mg/ml or higher. In particular embodiments, the GAL9 binding molecule is present at a concentration of more than 50 mg/ml.

In various embodiments, the pharmaceutical compositions are described in more detail in U.S. Pat. Nos. 8,961,964, 8,945,865, 8,420,081, 6,685,940, 6,171,586, 8,821,865, 9,216,219, U.S. application Ser. No. 10/813,483, WO 2014/066468, WO 2011/104381, and WO 2016/180941, each of which is incorporated herein in its entirety.

6.10. Methods of Treatment

In another aspect, methods of treatment are provided, the methods comprising administering a GAL9 binding molecule as described herein to a patient (e.g., subject) with a disease or condition in an amount effective (e.g., therapeutically effective amount) to treat the patient.

6.10.1. Subjects

In some embodiments, the subject is a mammal. In some embodiments, the mammal is a mouse. In a preferred embodiment, the mammal is a human. In some embodiments, the subject's immune cells have increased PD-L2 expression, relative to immune cells from healthy individuals (e.g., healthy control), such as blood dendritic cells.

6.10.2. Combination therapy

The GAL9 binding molecule can be used alone or in combination with other therapeutic agents or procedures to treat or prevent a disease or condition. The GAL9 binding molecule can be administered either simultaneously or sequentially dependent upon the disease or condition to be treated.

The anti-GAL9 binding molecules can be used in combination with an agent or procedure that is used in the clinic or is within the current standard of care to treat or prevent a disease or condition.

In some embodiments, the GAL9 binding molecule is administered in combination with a second immunosuppressive agent. In certain embodiments, the second immunosuppressive agent is a glucocorticoid (e.g., prednisone, dexamethasone, or hydrocortisone), a cytostatic, anti-cytokine antibodies including anti-TNFα, anti-IL1, anti-ILS, anti-IL-6, anti-IL-17 antibodies, and anti-IL-23 antibodies, and small molecule drugs that reduce inflammatory cytokine signaling, such as JAK/STAT inhibitors, methotrexate, hydroxychloroquine, chloroquine, an anti-CD25 or anti-CD52 antibody, or drugs acting on immunophilins (e.g., cyclosporine or Sirolimus, or any other drug known to inhibit or prevent activity of the immune system.

In some embodiments, the GAL9 binding molecule is administered in combination with one or more anti-inflammatory drugs.

6.10.3. Autoimmune or Inflammatory Diseases

In some embodiments, the treatment comprises administration of a GAL9 binding molecule as described herein to a subject with an autoimmune or inflammatory disease in an amount effective to treat the subject.

In some embodiments, the autoimmune disease is amyotrophic lateral sclerosis (ALS), achalasia, Addison's disease, adult still's disease, agammaglobulinemia, alopecia areata, amyloidosis, ankylosing spondylitis, anti-GBM/anti-TBM nephritis, Antiphospholipid syndrome, autoimmune angioedema, autoimmune dysautonomia, autoimmune encephalomyelitis, autoimmune hepatitis, autoimmune inner ear disease, autoimmune myocarditis, autoimmune oophoritis, autoimmune orchitis, autoimmune pancreatitis, autoimmune retinopathy, autoimmune urticaria, axonal & neuronal neuropathy (AMAN), Baló disease, Behcet's disease, benign mucosal pemphigoid, bullous pemphigoid, castleman disease, celiac disease, Chagas disease, chronic inflammatory demyelinating polyneuropathy, chronic recurrent multifocal osteomyelitis, Churg-Strauss Syndrome, Eosinophilic Granulomatosis, Cicatricial pemphigoid, Cogan's syndrome, cold agglutinin disease, congenital heart block, coxsackie myocarditis, CREST syndrome, Crohn's disease, dermatitis herpetiformis, dermatomyositis, Devic's disease (neuromyelitis optica), discoid lupus, dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, goodpasture's syndrome, granulomatosis with polyangiitis, Graves' disease, Guillain-Barre syndrome, Hashimoto's thyroiditis, hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis, Interstitial cystitis, Juvenile arthritis, Juvenile diabetes (Type 1 diabetes), Juvenile myositis, Kawasaki disease, Lambert-Eaton syndrome, Leukocytoclastic vasculitis, Lichen planus, Lichen sclerosus, Ligneous conjunctivitis, Linear IgA disease (LAD), lupus, lyme disease chronic, Meniere's disease, microscopic polyangiitis, mixed connective tissue disease (MCTD), Mooren's ulcer, Mucha-Habermann disease, Multifocal Motor Neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, pemphigus, peripheral neuropathy, perivenous encephalomyelitis, pernicious anemia (pa), POEMS syndrome, polyarteritis nodosa, polyglandular syndromes type I, II, or III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia, pyoderma gangrenosum, Raynaud's phenomenon, reactive arthritis, reflex sympathetic dystrophy, relapsing polychondritis, restless legs syndrome, retroperitoneal fibrosis, rheumatic fever, rheumatoid arthritis, sarcoidosis, Schmidt syndrome, scleritis, scleroderma, Sjögren's syndrome, sperm & testicular autoimmunity, stiff person syndrome, subacute bacterial endocarditis, Susac's syndrome, sympathetic ophthalmia, Takayasu's arteritis, temporal arteritis, giant cell arteritis, thrombocytopenic purpura, Tolosa-Hunt syndrome, transverse myelitis, type 1 diabetes, ulcerative colitis, undifferentiated connective tissue disease, uveitis, vasculitis, vitiligo, or Vogt-Koyanagi-Harada disease.

In some embodiments, the autoimmune disease is selected from the group consisting of: inflammatory bowel disease, Crohn's disease, ulcerative colitis, colitis, celiac disease, rheumatoid arthritis, Behçet's disease, amyloidosis, psoriasis, psoriatic arthritis, systemic lupus erythematosus nephritis, graft-versus-host disease (GVHD), nonalcoholic steatohepatitis (NASH), and ankylosing spondylitis. In a preferred embodiment, the disease is Crohn's Disease.

In some embodiments, the treatment comprises administration of a GAL9 binding molecule as described herein to a subject at risk for transplantation rejection in an amount effective to reduce transplant rejection. In some embodiments, the treatment comprises administration of a GAL9 binding molecule as described herein to a subject with graft-versus-host disease in an amount effective to reduce GvHD. In some embodiments, the treatment comprises administration of a GAL9 binding molecule as described herein to a subject with post-traumatic immune responses in an amount effective to reduce inflammation. In some embodiments, the treatment comprises administration of a GAL9 binding molecule as described herein to a subject with ischemia in an amount effective to treat the subject. In some embodiments, the treatment comprises administration of a GAL9 binding molecule as described herein to a subject who has undergone a stroke in an amount effective to treat the subject.

In some embodiments, the treatment comprises administration of a GAL9 binding molecule to a subject who has a viral infection in an amount effective to reduce acute respiratory distress syndrome and/or acute cytokine release syndrome (cytokine storm). In particular embodiments, the viral infection is infection with SARS-CoV-2 virus and the disease is COVID-19.

6.10.4. Administration

The GAL9 binding molecule may be administered to a subject by any route known in the art. For example, the GAL9 binding molecule may be administered to a human subject via, e.g., intraarterial, intramuscular, intradermal, intravenous, intraperitoneal, intranasal, parenteral, pulmonary, subcutaneous administration, topical, oral, sublingual, intratumoral, peritumoral, intralesional, intrasynovial, intrathecal, intra-cerebrospinal, or perilesional administration. The GAL9 binding molecule may be administered to a subject per se or as a pharmaceutical composition. Exemplary pharmaceutical compositions are described herein.

The anti-GAL9 binding molecules disclosed herein can be administered alone or in combination with other therapeutic agents or procedures to treat or prevent a disease or condition.

Depending on the condition or disease to be treated, the treatment with a GAL9 binding molecule can improve one or more clinical endpoints in a subject. Examples of clinical endpoints improved in a subject with a disease or condition include but are not limited to, reducing inflammation, reducing autoimmune response, prolonging remission, inducing remission, re-establishing immune tolerance, improving organ function, reducing the risk of progression or development of a disease or a condition, reducing the risk of progression or development of a second disease, increasing overall survival in the subject or a combination thereof.

### 6.11. EXAMPLES

The following examples are provided by way of illustration, not limitation. In particular, methods for the expression and purification of the various antigen-binding proteins and their use in various assays described below are non-limiting and illustrative.

6.11.1. Methods

6.11.1.1. Expi293 Expression

Various antigen-binding proteins tested were expressed using the Expi293 transient transfection system according to manufacturer's instructions. Briefly, plasmids coding for individual chains were mixed at 1:1 mass ratio, unless otherwise stated, and transfected into Expi 293 cells with ExpiFectamine 293 transfection kit. Cells were cultured at 37° C. with 8% CO₂, 100% humidity and shaking at 125 rpm. Transfected cells were fed once after 16-18 hours of transfections. The cells were harvested at day 5 by centrifugation at 2000 g for 10 minutes. The supernatant was collected for affinity chromatography purification.

6.11.1.2. ExpiCHO Expression

Various GALS antigen-binding proteins are expressed using the ExpiCHO transient transfection system according to manufacturer's instructions. Briefly, plasmids coding for individual chains are mixed at, for example, a 1:1 mass ratio, and transfected with ExpiFectamine CHO transfection kit into ExpiCHO.

Cells are cultured at 37° C. with 8% CO₂, 100% humidity and shaking at 125 rpm. Transfected cells are generally be fed once after 16-18 hours of transfections. The cells are harvested at day 5 by centrifugation at 2000 g for 10 munities. The supernatant is then collected for affinity chromatography purification.

6.11.1.3. Protein A Purification

Cleared supernatants containing the various antigen-binding proteins were separated using either a Protein A (ProtA) resin or an anti-CH1 resin on an Gravity flow purifier. In examples where a head-to-head comparison was performed, supernatants containing the various antigen-binding proteins were split into two equal samples. For ProtA purification, a 1 mL Protein A column (GE Healthcare) was equilibrated with PBS (5 mM sodium potassium phosphate pH 7.4, 150 mM sodium chloride). The sample was loaded onto the column at 5 ml/min. The sample was eluted using 0.1M Sodium acetate pH 3.5. The elution was monitored by absorbance at 280 nm and the elution peaks were pooled for analysis. The elution was monitored by absorbance at 280 nm and the elution peaks were pooled for analysis.

6.11.1.4. SDS-Page Analysis

Samples containing the various separated antigen-binding proteins were analyzed by reducing and non-reducing SDS-PAGE for the presence of complete product, incomplete product, and overall purity. 2 μg of each sample was added to 15 μL SDS loading buffer. Reducing samples were incubated in the presence of 10 mM reducing agent at 75° C. for 10 minutes. Non-reducing samples were incubated at 70° C.—for 5 minutes without reducing agent. The reducing and non-reducing samples were loaded into a 4-15% gradient TGX gel (BioRad) with running buffer and run for 30 minutes at 220 volts. Upon completion of the run, the gel was washed with DI water and stained using GelCode Blue Safe Protein Stain (ThermoFisher). The gels were destained with DI water prior to analysis. Densitometry analysis of scanned images of the destained gels was performed using standard image analysis software to calculate the relative abundance of bands in each sample.

6.11.1.5. IEX Chromatography

Samples containing the various separated antigen-binding proteins were analyzed by cation exchange chromatography for the ratio of complete product to incomplete product and impurities. Cleared supernatants were analyzed with a 5-ml MonoS (GE Lifesciences) on an AKTA Purifier FPLC. The MonoS column was equilibrated with buffer A 10 mM MES pH 6.0. The samples were loaded onto the column at 2 ml/min. The sample was eluted using a 0-30% gradient with buffer B (10 mM MES pH 6.0, 1 M sodium chloride) over 6 CV. The elution was monitored by absorbance at 280 nm and the purity of the samples were calculated by peak integration to identify the abundance of the monomer peak and contaminants peaks. The monomer peak and contaminant peaks were separately pooled for analysis by SDS-PAGE as described above.

Analytical SEC Chromatography of each sample at 1 mg/mL was loaded onto the column at 1 ml/min. The sample was eluted using an isocratic flow of PBS for 1.5 CV. The elution was monitored by absorbance at 280 nm and the elution peaks were analyzed by peak integration.

6.11.1.6. Mass Spectrometry

Samples containing the various separated antigen-binding proteins were analyzed by mass spectrometry to confirm the correct species by molecular weight. All analysis was performed by a third-party research organization. Briefly, samples were treated with a cocktail of enzymes to remove glycosylation. Samples were both tested in the reduced format to specifically identify each chain by molecular weight. Samples were all tested under non-reducing conditions to identify the molecular weights of all complexes in the samples. Mass spec analysis was used to identify the number of unique products based on molecular weight.

6.11.1.7. Antibody discovery by phage display

Phage display of human Fab libraries was carried out using standard protocols. Human GAL9 protein was purchased from Acro Biosystems (Human Gal9 His-tag Cat #LG9-H5244) and biotinylated using EZ-Link NHS-PEG12-Biotin (ThermoScientific Cat #21312) using standard protocols. Phage clones were screened for the ability to bind the GAL9 protein by phage ELISA using standard protocols.

Briefly, Fab-formatted phage libraries were constructed using expression vectors capable of replication and expression in phage (also referred to as a phagemid). Both the heavy chain and the light chain were encoded for in the same expression vector, where the heavy chain was fused to a truncated variant of the phage coat protein pIII. The light chain and heavy chain-pIII fusion were expressed as separate polypeptides and assembled in the bacterial periplasm, where the redox potential enables disulfide bond formation, to form the phage display antibody containing the candidate ABS.

The library was created using sequences derived from a specific human heavy chain variable domain (VH3-23) and a specific human light chain variable domain (W-1). For the screened library, all three CDRs of the VH domain were diversified to match the positional amino acid frequency by CDR length found in the human antibody repertoire. Light chain variable domains within the screened library were generated with diversity introduced solely into the VL CDR3 (L3); the light chain VL CDR1 (L1) and CDR2 (L2) retained the human germline sequence.

The heavy chain scaffold (SEQ ID NO:2), light chain scaffold (SEQ ID NO:4), full heavy chain Fab polypeptide (SEQ ID NO:1), and full light chain Fab polypeptide (SEQ ID NO:3) used in the phage display library are shown below, where a lower case “x” represents CDR amino acids that were varied to create the library.

Phage display VH scaffold [SEQ ID NO: 2]:
EVQLVESGGGLVQPGGSLRLSCAASGFTFxxxxIHWVRQAPGKGLEWVA
xxxxxxxxxxxYADSVKGRFTISADTSKNTAYLQMNSLRAEDTAVYYCA
RxxxxxxxxxxxxxDYWGQGTLVTVSSAS
Phage display VL scaffold [SEQ ID NO: 4]:
DIQMTQSPSSLSASVGDRVTITCRASQSVSSAVAWYQQKPGKAPKLLIY
SASSLYSGVPSRFSGSRSGTDFTLTISSLQPEDFATYYCQQxxxxxxTF
GQGTKVEIKRT
Phage display heavy chain Fab polypeptide [SEQ
ID NO: 1]:
EVQLVESGGGLVQPGGSLRLSCAASGFTFxxxxIHWVRQAPGKGLEWVA
xxxxxxxxxxxYADSVKGRFTISADTSKNTAYLQMNSLRAEDTAVYYCA
RxxxxxxxxxxxxxDYWGQGTLVTVSSASTKGPSVFPLAPSSKSISGGT
AALGCLVKDYFPEPVTVSWNSGALTSGVHTFPAVLQSSGLYSLSSVVTV
PSSSLGTQTYICNVNHKPSNTKVDKKVEPKSCDKTHTC
Phage display light chain Fab polypeptide [SEQ
ID NO: 3]:
DIQMTQSPSSLSASVGDRVTITCRASQSVSSAVAWYQQKPGKAPKLLIY
SASSLYSGVPSRFSGSRSGTDFTLTISSLQPEDFATYYCQQxxxxxxTF
GQGTKVEIKRTVAAPSVFIFPPSDSQLKSGTASVVCLLNNFYPREAKVQ
WKVDNALQSGNSQESVTEQDSKDSTYSLSSTLTLSKADYEKHKVYACEV
THQGLSSPVTKSFNRGEC

Diversity was created through Kunkel mutagenesis using primers to introduce diversity into VH CDR1 (H1), CDR2 (H2) and CDR3 (H3) and VL CDR3 to mimic the diversity found in the natural antibody repertoire, as described in more detail in Kunkel, T A (PNAS Jan. 1, 1985. 82 (2) 488-492), incorporated herein by reference in its entirety. Briefly, single-stranded DNA was prepared from isolated phage using standard procedures and Kunkel mutagenesis carried out. Chemically synthesized DNA was then electroporated into MC1061F-cells. Phagemid obtained from overnight culture was digested with restriction enzymes (Bam HI and Xba I) to remove the wild-type sequence. The digested sample was electroporated into TG1 cells, followed by recovery. Recovered cells were sub-cultured and infected with M13K07 helper phage to produce the phage library.

Phage panning was performed using standard procedures. Briefly, the first round of phage panning was performed with target immobilized on streptavidin magnetic beads which were subjected to ˜5×10¹² phages from the prepared library in a volume of 1 mL in PBST-2% BSA. After a one-hour incubation, the bead-bound phage were separated from the supernatant using a magnetic stand. Beads were washed three times to remove non-specifically bound phage and were then added to ER2738 cells (5 mL) at OD₆₀₀˜0.6. After 20 minutes, infected cells were sub-cultured in 25 mL 2×YT⁺ Ampicillin and M13K07 helper phage (final concentration, ˜10¹⁰ pfu/ml) and allowed to grow overnight at 37° C. with vigorous shaking. The next day, phage were prepared using standard procedures by PEG precipitation. Pre-clearance of phage specific to SAV-coated beads was performed prior to panning. The second round of panning was performed using the KingFisher magnetic bead handler with 100 nM bead-immobilized antigen using standard procedures. In total, 3-4 rounds of phage panning were performed to enrich in phage displaying Fabs specific for the target antigen. Target-specific enrichment was confirmed using polyclonal and monoclonal phage ELISA. DNA sequencing was used to determine isolated Fab clones containing a candidate ABS.

The VL and VH domains identified in the phage screen described above were reformatted into a bivalent monospecific native human full-length IgG1 architecture.

Native human full-length IgG1 heavy chain
architecture [SEQ ID NO: 5]:
[SEQ ID NO: 5]
EVQLVESGGGLVQPGGSLRLSCAASGFTFxxxxIHWVRQAPGKGLEWVA
xxxxxxxxxxxYADSVKGRFTISADTSKNTAYLQMNSLRAEDTAVYYCA
RxxxxxxxxxxxxxDYWGQGTLVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALH
NHYTQKSLSLSPGK

Native Human Full-Length IgG1 Light Chain Architecture:

Equivalent to phage display light chain Fab, see SEQ ID NO:3

6.11.1.8. Octet Determination of Binding Kinetics

To measure qualitative binding affinity in GAL9 binder discovery campaigns, IgG1 reformatted binders were immobilized to a biosensor on an Octet (Pall ForteBio) biolayer interferometer.

Soluble GAL9 antigen was then added to the system and binding measured. Qualitative binding affinity was assessed by visualizing the slope of the dissociation phase of the octet sensogram from weakest (⁺) to strongest (⁺⁺⁺). A slow off rate represented by a negligible drop in the dissociation phase of the sensogram and indicated a tight binding antibody (⁺⁺⁺). To obtain accurate kinetic constants for monovalent affinities, a dilution series involving of at least five concentrations of the GAL9 analyte (ranging from approximately 10 to 20× K_{D} to 0.1× K_{D} value, 2-fold dilutions) were measured in the association step. In the dissociation step, the sensor was dipped into buffer solution that did not contain the GAL9 analyte and where the bound complex on the surface of the sensor dissociates. Octet kinetic analysis software was used to calculate the kinetic and equilibrium binding constants based on the rate of association and dissociation curves. Analysis was performed globally (global fit) where kinetic constants were derived simultaneously from all analyte concentration included in the experiment.

6.11.1.9. Epitope Binning

Anti-GAL9 candidates formatted into a bivalent monospecific native human full-length IgG1, as described above, were tested for GAL9 binding in a pair-wise manner using an octet-based ‘tandem’ assay. Briefly, biotinylated GAL9 was immobilized on a streptavidin sensor and two anti-GAL9 candidates were bound in tandem. A competitive blocking profile was generated determining whether a given anti-GAL9 candidate blocked binding of a panel of other anti-GAL9 candidates to GAL9. Anti-GAL9 candidates that competed for the same or non-overlapping binding regions were grouped together and referred to as belonging to the same bin.

6.11.1.10. PBMC Activation and Galectin 9 Antibody Treatment

Individual aliquots of PepMix HCMVA (pp65) (>90%) Protein ID: P06725 (Cat. No. PM-PP65-2, JPT Peptide Technologies) were prepared according to manufacturer's instructions. PepMix™ HCMVA (pp65) are complete protein-spanning mixtures of overlapping 15mer peptides through 65 kDa phosphoprotein (pp65) (Swiss-Prot ID: P06725) of Human cytomegalovirus (HHV-5), used for immunostimulation of immune cell responses.

Frozen human peripheral blood mononuclear cells (PBMCs) were thawed according to standard conditions, then resuspended in growth media (10% FBS in RPMI).

Resuspended PBMCs were seeded at 5×10⁵ cells in 96-well plates. Cells were incubated with 2 μg/mL PepMix™ HCMVA (pp65) plus 40 μg/mL of candidate GAL9 antibodies or control antibodies in growth media for 24 hours at 37° C., 5% CO₂.

6.11.1.11. LEGENDplex Human Th Cytokine Assay

Following PBMC activation and Galectin 9 antibody treatment as described herein, cytokine secretion by PBMCs and immune cell subpopulations was assessed at 24 hours and 72 hours post-treatment by cytokine bead array as follows.

200 μl cell culture supernatant was collected and centrifuged to pellet cell debris. The resulting supernatants were analyzed using the LEGENDplex™ Human Th1 Panel (5-plex) (Cat. No. 740009, Biolegend). The LEGENDplex™ Human Th1 Panel is a bead-based assay to allows for simultaneous quantification of human cytokines IL-2, IL-6, IL-10, IFN-γ and TNF-α using flow cytometry.

Briefly, cytokine standards and capture bead mixtures were prepared according to manufacturer's instructions. Assay master mixes of 1:1:1 capture bead mixture: biotinylated detection antibodies; assay buffers were prepared.

12.5 μl of supernatant samples or cytokine standards were incubated with 37.5 μl assay master mix. Plates were sealed, covered with foil, and shaken at 600 rpm for 2 hours at room temperature. Wells were then incubated, with shaking at 600 rpm, with streptavidin-phycoerythrin (SA-PE) for 30 minutes at room temperature. Beads were then washed twice and resuspended before proceeding to flow cytometry analysis according to manufacturer's instructions.

6.11.1.12. PBMC Staining with Marker Antibodies

Following PBMC activation and Galectin 9 antibody treatment as described herein, PBMCs immune cells were stained with marker antibodies according to the following procedures.

Cells were resuspended at 5×10⁶ cells/mL in growth media (10% FBS in RPMI). 200 μL of resuspended cells were aliquoted to 96 well plates, then incubated with Fixable Viability Dye eFluor® 780 for 30 minutes at 2-8° C. to irreversibly label dead cells. Cells were then washed and then incubated with human Fc Block solution (Cat. No. 14-9161-73, eBiosciences) for 10 minutes at room temperature.

An antibody cocktail working solution was prepared according to the following table.

TABLE 1
Antibody Staining Working Solutions

| | Antibody | Dilution |
|---|---|---|
| T cell surface markers | BV510 anti-human CD3 (Cat. No. | 1 in 20 |
| | 563109, BD Biosciences) | |
| | PerCP/Cy5.5 anti-human CD56 (Cat. | 1 in 20 |
| | No. 362505, BD Biosciences) | |
| Monocyte surface markers | FITC anti-human CD14 (Cat. No. | 1 in 20 |
| | 367115, BD Biosciences) | |
| | Alexa Fluor ® 700 anti-human CD16 | 1 in 20 |
| | (Cat. No. 302025, Biolegend) | |
| Dendritic cell surface | Brilliant Violet 421 ™ anti-human | 1 in 20 |
| makers | CD11c (Cat. No. 301627, Biolegend) | |
| | Alexa Fluor 647 anti-human CD123 | 1 in 40 |
| | (Cat. No. 306023, Biolegend) | |
| | BV510 anti-human Lineage Cocktail | 1 in 10 |
| | (CD3, CD14, CD16, CD19, CD20, | |
| | CD56) (Cat. No. 348807, Biolegend) | |
| | FITC anti-human HLA-DR (Cat. No. | 1 in 20 |
| | 307603, Biolegend) | |
| B cell surface markers | PerCP/Cy5.5 anti-human CD19 (Cat. | 1 in 20 |
| | No. 363015, Biolegend) | |
| Galectin-9 | PE anti-human galectin 9 (Cat. No. | 1 in 10 |
| | 348905, Biolegend) | |

Wells were incubated with 10 μL of diluted antibody cocktail for 30 minutes at 2-8° C. Cells were then washed and resuspended and analyzed by flow cytometry analysis.

To analyze immune stimulatory markers CD27, CD40L, ICOS, 4-1BB, and OX40, the same protocol provided above was followed, but cells were incubated with the alternative antibody cocktail as detailed in Table 2 below:

TABLE 2
Antibody Staining Working Solutions

| Antibody | Dilution |
|---|---|
| FITC anti-human CD134 (OX40) (Cat. No. | 1 in 50 |
| 350006, BioLegend) | |
| PerCP/Cy5.5 anti-human CD3 (Cat. No. | 1 in 100 |
| 560835, BD Biosciences) | |
| AF700 anti-human CD4 (Cat. No. 344622, | 1 in 100 |
| BioLegend) | |
| eFluor ™ Fixable Viability Dye (Cat. No. | 1 in 2000 |
| 65-0865-14, eBioscienceTM) | |
| BV421 anti-human CD8 (Cat. No. 344748, | 1 in 100 |
| BioLegend) | |
| BV650 anti-human CD137 (4-1BB) (Cat. | 1 in 50 |
| No. 309828, BioLegend) | |
| BV711 anti-human ICOS (Cat. No. 563833, | 1 in 100 |
| BD Biosciences) | |
| PE anti-human CD154 (CD40L) (Cat. No. | 1 in 50 |
| 310806, BioLegend) | |
| PE/Cy7 anti-mouse/rat/human CD27 (Cat. | 1 in 100 |
| No. 124216, BioLegend) | |

### 6.11.2. Example 1: Blood Dendritic Cells from Crohn's Disease Patients have Increased PD-L2 Expression

Programmed death 1 (PD-1)-deficient mice develop a variety of autoimmune-like diseases, which suggests that the PD-1 receptor plays an important role in immunity and autoimmunity. PD-1 has two endogenous ligands, PD-L1 and PD-L2. The PD-1/PD-L1 interaction has been implicated in autoimmunity; however, PD-L2's role in autoimmunity is less understood.

Crohn's disease (CD) is a chronic inflammatory disease of the gastrointestinal tract. While the specific cause of the disease is not well understood, it is clear that CD patients have an overactive immune system that causes inflammation and damage to the gastrointestinal tract. This study was conducted to determine the expression of PD-L2 and PD-L1 on blood dendritic cells from Crohn's Disease patients.

Study Participants

Peripheral blood was drawn from 29 adults confirmed by colonoscopy to have Crohn's disease. Patients were selected at different stages of treatment, but were excluded if they had received anti-TNF-α treatment. For a control, peripheral blood was drawn from 13 healthy adults undergoing colorectal cancer family history screening.

Immunostaining

Single-cell suspensions obtained from 10 ml whole blood were incubated with an Fc receptor binding antibody to block nonspecific Fc binding by specific antibodies. Fixable Viability Dye eFluor780 (ebioscience, San Diego, Calif.) was used to exclude dead cells from analysis. The following anti-human monoclonal antibodies were used to assess cells: HLA-DR PerCP-Cy5.5 (clone G46-6; BD Bioscience, San Jose, Calif.); lineage cocktail BV510 [CD3 (clone OKT3)/CD14 (clone M5E2)/CD16 (clone 3G8)/CD19 (clone HIB19)/CD20 (clone 2H7) and CD56 (clone HCD56)]; CD11c BV605 (clone 3.9; BioLegend, San Diego, Calif.).

Anti-human PD-L2 monoclonal antibody (clone MIH18; BioLegend, San Diego, Calif.) and anti-human PD-L1 monoclonal antibody (clone 29E.2A3; BioLegend, San Diego, Calif.) or control IgGs were labelled in-house using the Lightning-Link Rapid DyLight 647 and Lightning-Link Rapid DyLight 488, respectively (BioNovus Life Sciences, Cherrybrook, NSW, Australia). Cells were stained with anti-HLA-DR, anti-PD-L2, or anti-PD-L1 or IgG control for 30 mins at room temperature, and then washed twice with PBS for 5 mins, and then fixed in 1% paraformaldehyde—PBS, pH 7.25.

Flow Cytometry

Cells were stained with Fixable Viability Dyes (FVD) and gated to capture only viable cells in the mononuclear cell region of a side scatter versus forward scatter plot. Dendritic cells were defined as HLA-DR⁺ and Lint, followed by gating CD11c⁺ within the total peripheral blood population. For each donor at least 1×10⁴ events were collected.

Cells were analyzed using a BD LSR Fortessa flow cytometer and data analyzed using either BD FACSDiva software (Becton & Dickinson, Franklin Lakes, N.J.), FCS express (De Novo software, Glendale, Calif.) or FlowJo software (Tree Star; a subsidiary of Becton, Dickinson and Company, Ashland, Oreg.).

Statistical Analyses

Non-parametric Mann-Whitney U test based on 2-sided tail was conducted using GraphPad Prism (GraphPad Software).

Microscopy

Microscopy samples were made by mounting stained, sorted cells onto a glass slide. Images were collected using a confocal microscope.

Results/Conclusion

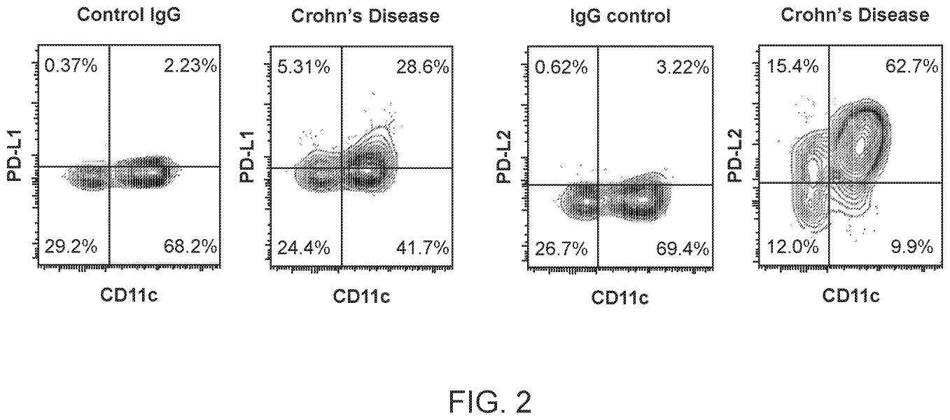

FIG. 2 shows contour plots of CD11c⁺ dendritic cells (DCs) cells from Crohn's patients stained with either IgG control, anti-PD-L1, or anti-PD-L2. We observed that the IgG control had 2.23% non-specific binding to DC cells, whereas the anti-PD-L1 antibody stained 28.6% of DC cells as PD-L1⁺. Likewise, in the second experiment, the IgG control bound to only 3.22% of CD11c⁺ DC, whereas the anti-PD-L2 antibody detected 62.7% of DC cells as PD-L2⁺.

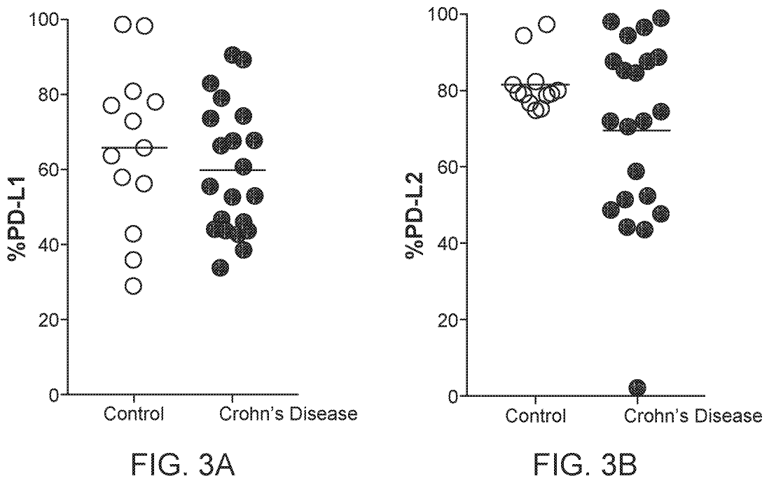

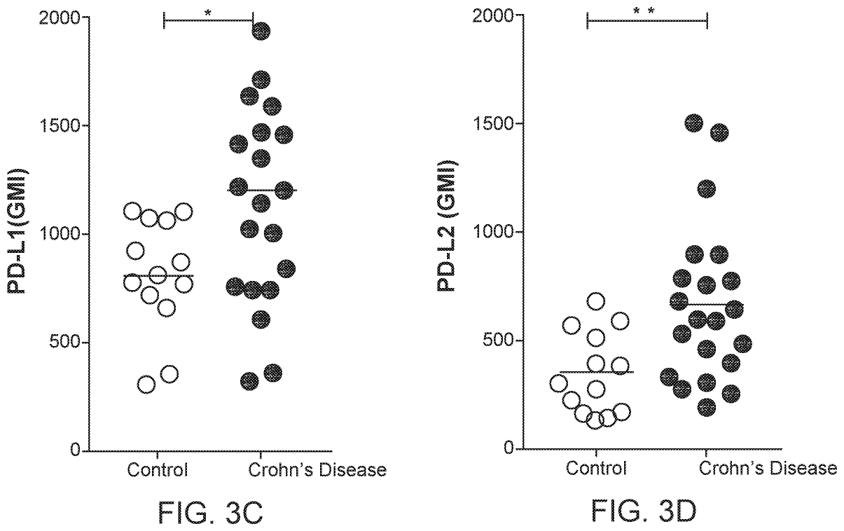

FIGS. 3A-3B show scatter plots of the percentage of PD-L1+ cells among CD11c⁺ blood dendritic cells (FIG. 3A) and the percentage of PD-L2⁺ cells among CD11c⁺ blood dendritic cells (FIG. 3B) from healthy control donors and CD patients. The horizontal bars on the scatter plots show the mean. FIGS. 3C-3D show scatter plots of the amount (GMI) of PD-L1 expression (FIG. 3C) and the amount (GMI) of PD-L2 expression on CD11c⁺ blood dendritic cells from healthy control donors and Crohn's patients (FIG. 3D). The horizontal bars on the scatter plots indicate the mean. A single asterisk “*” indicates a P-value=0.0292. A double asterisk “**” indicates a P-value=0.0032.

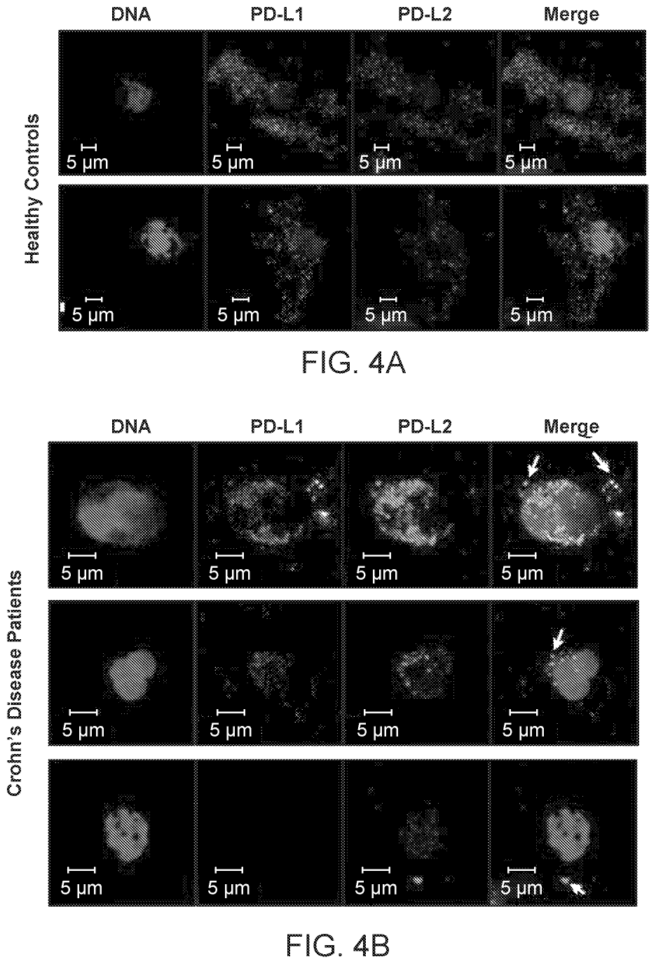

FIGS. 4A-4B show representative immunostaining of dendritic cells (DC) cells from the blood of two healthy control donors and three Crohn's Disease patients. DCs from healthy controls show high PD-L1 (green) and PD-L2 (red) staining throughout the cell; rendered in gray scale in the attached figures. In contrast, dendritic cells from Crohn's patients show low PD-L1 expression and high levels of PD-L2 which appear aggregated. In some cells, we observed high staining of aggregated PD-L1.

The results demonstrate that the PD-L2 protein is more highly expressed in blood dendritic cells from Crohn's patients as compared to healthy control donors (P-value=0.0032), yielding a higher statistical difference than PD-L1 (P-value=0.0292). These results suggest that the PD-L2 pathway may play an important role in Crohn's Disease and other autoimmune diseases.

### 6.11.3. Example 2: Inhibiting PD-L2 in PBMCs from Crohn's Disease Patients Results in a Clinically Favorable Cytokine Profile

This study was conducted to determine the effect of inhibiting PD-L2 protein on the cytokine profile in PBMCs from Crohn's Disease (CD) patients, compared to an IgG control.

Study Participants

Blood samples were obtained from 14 different Crohn's disease patients. Peripheral blood mononuclear cells (PBMC) were isolated using heparinized blood by density centrifugation on Ficoll-Paque (Pharmacia, Freiburg, Germany). Isolated PBMCs from control and CD patients were added to wells (2×10⁵ cells/well) pre-coated with anti-CD3. R10 media, supplemented with penicillin (100 IU/ml), streptomycin (0.1 mg/ml) and L-glutamine (0.29 gm/1). Control IgG or blocking anti-PD-L2 (MIH18) antibodies were added to the culture at 20 μg/ml.

Treatment

Matched PBMCs samples were treated with either IgG control or anti-human PD-L2 antibody clone MIH18 (BioLegend) for 36 hours and then assayed.

Cytokine Assay

The concentration of TNF-α, IFN-γ, and IL-10 were measured using BD™ Cytometric Bead Array (CBA) following manufacturer's instructions.

Statistical Analyses

Wilcoxon matched-pairs signed rank test was conducted using GraphPad Prism (GraphPad Software).

Results/Conclusion

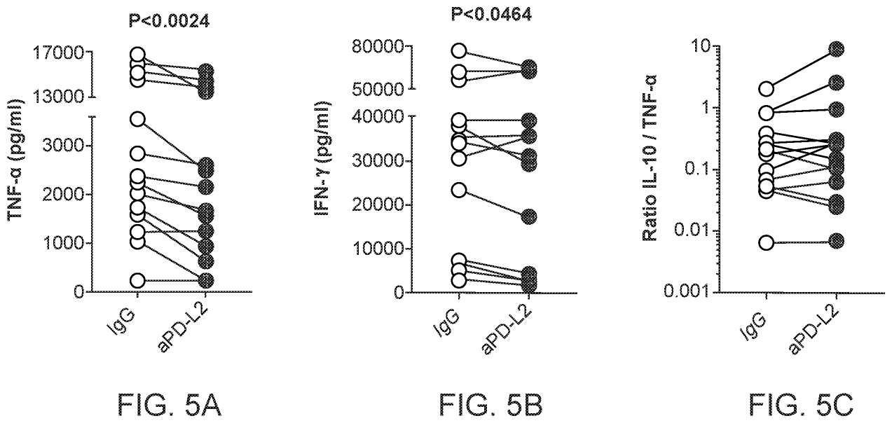

The mean concentrations of TNF-α and IFN-γ from the matched samples are shown in FIGS. 5A-5B, respectively. FIG. 5C shows the mean IL-10:TNF-α ratio. These results demonstrate that inhibiting PD-L2 results in a clinically favorable cytokine profile in PMBCs from CD patients, by decreasing the levels of pro-inflammatory cytokines TNF-α and IFN-γ, and increasing the levels of inhibitory cytokine IL-10.

### 6.11.4. Example 3: Stimulating or Blocking the GAL9/PD-L2 Pathway Modulates TNF-α Secretion in Mouse CD4⁺ T Cells

Previously, we showed that GAL9 can bind soluble PD-L2, and that some of the immunological effects of PD-L2 are mediated through binding of multimeric PD-L2 to GAL9, rather than through PD-1/PD-L1 (WO 2016/008005, which is incorporated herein by reference in its entirety). The current study was conducted to determine if stimulating or blocking the GAL9/PD-L2 pathway can modulate the TNF-α secretion in mouse CD4⁺ T cells.

Animals

C57BL6/J mice were used for the study. All animals used in the study were housed and cared for in accordance with the National Health Medical Research Council (NHMRC) Guidelines for Animal Use.

sPD-L2

Soluble mouse PD-L2 (sPD-L2) with a human IgG1 Fc was custom produced by Geneart (Germany).

Antibodies

For treatment, inhibitory anti-mouse GAL9 antibody clone 108A2 (BioLegend® San Diego, Calif.) or rat IgG2a control antibody was used. The anti-mouse GAL9 clone (108A2) binds the linker peptide of murine Galectin-9 (Oomizu, S. et al., PLoS One 7(11):e48574 (2012); Doi: 10.1371/journal.pone.0048574, which is herein incorporated by reference). Anti-CD3 (clone 145.2C11) (Aviva Systems Biology Corp. San Diego, Calif.) was used for stimulation.

Cell Separation and Stimulation of CD4⁺ T cells

A suspension of mouse spleen cells was made from five mice. CD4⁺ T-cells were isolated using Miltenyi Biotec Inc. (Auburn, Calif.) kit for untouched CD4⁺ T cells. Mouse CD4⁺ T cells were stimulated with anti-CD3 clone 145.2C11 (Aviva Systems Biology Corp. San Diego, Calif.) at 5 μg/ml. Next, the stimulated CD4⁺ T cells were treated either with IgG control or sPD-L2 at 20 μg/ml, or with sPD-L2 and anti-GAL9 mAb clone 108A2, both at 20 μg/ml, and then cultured for 36 hours.

Cytokine Assays

After 36 hrs of treatment, the concentration of TNF-α was measured using BD™ Cytometric Bead Array following manufacturer's instructions.

Statistical Analyses

Non-parametric Mann-Whitney U test was conducted using GraphPad Prism (GraphPad Software).

Results/Conclusion

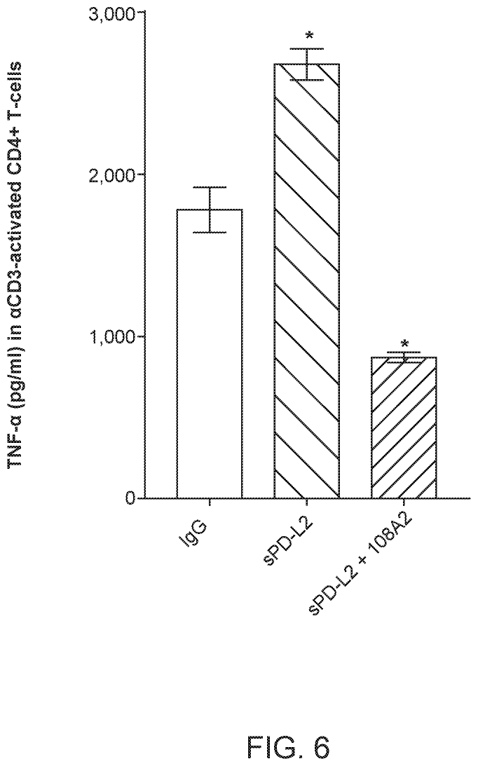

FIG. 6 shows bar graphs of the concentration levels of TNF-α for each treatment group. Treatment of activated CD4⁺ T cells with sPD-L2 alone resulted in significantly increased TNF-α secretion by CD4⁺ T cells, as compared to IgG control, * p-value <0.0001. Addition of inhibitory anti-mouse GAL9 antibody (108A2) significantly decreased TNF-α secretion from activated CD4⁺ T cells, both as compared to activated CD4⁺ T cells treated with 108A2, and as compared to IgG control, * p-value <0.0001.

sPD-L2, which binds GAL9 on T cells, induces TNF-α secretion, while inhibiting GAL9 blocks sPD-L2-mediated TNF-α secretion in CD4⁺ T cells. These results demonstrate that the GAL9/PD-L2 pathway modulates TNF-α levels in stimulated CD4⁺ T cells.

### 6.11.5. Example 4: Inhibitory Anti-Mouse GAL9 (108A2) Antibodies Works Independently from PD-1/PD-L1 in CD4⁺ T Cells from Malaria-Infected Mice, while Activating Anti-GAL9 Antibodies do not

This study was conducted to investigate the dependence of inhibitory and activating GAL9 antibodies on the PD-1/PD-L1 pathway.

Mouse models of malaria-infected mice can be used to study immune mechanisms and susceptibility to drugs. Wykes, M N et al. Eur J Immunol. (2009) 39:2004-7, which is incorporated herein by reference in its entirety. Further, it has been shown that Plasmodium parasites that cause malaria can exploit the PD-1 pathway to ‘deactivate’ T cell functions. A definitive role for PD-1 in malarial pathogenesis was demonstrated when PD-1-deficient mice were shown to rapidly and completely clear P. chabaudi infections. As such, malarial infection models can be used to understand the relative contribution of PD-1 and its ligands, PD-L1 and PD-L2, in immunity.

Antibodies

The inhibitory anti-mouse GAL9 antibody (108A2) and the activating anti-mouse GAL9 antibody (RG9.1) (Cat. No. BE0218, InVivoMab Antibodies) were used for this study.

Malaria-Infected Mouse Model

Cohorts of C57BL/6 mice were infected with non-lethal malaria (P. yoelii 17XNL). After intravenous injection the of 10⁵ P. yoelii infected red cells, the mice were incubated for 7 days to allow infection to take place.

CD4⁺ T Cell Isolation and Treatment

CD4⁺ T cells were isolated from malaria-infected mice using Miltenyi Biotec untouched CD4⁺ T cell isolation kits. Next, the isolated T cells were cultured and treated overnight with either control IgG antibody, inhibitory anti-mouse GAL9 antibody (108A2), or the activating anti-mouse GAL9 antibody (RG9.1).

Immunostaining and Microscopy

After treatment, the cells were stained with DAPI (to detect DNA), and anti-OX40 (CD134), anti-PD-1, and anti-PD-L1 (BioXCell, Lebanon, N.H.) antibodies labelled using Lightning-Link Rapid DyLight 647, 594 or 488 kits. Immunostaining was observed by confocal imaging.

Results/Conclusion

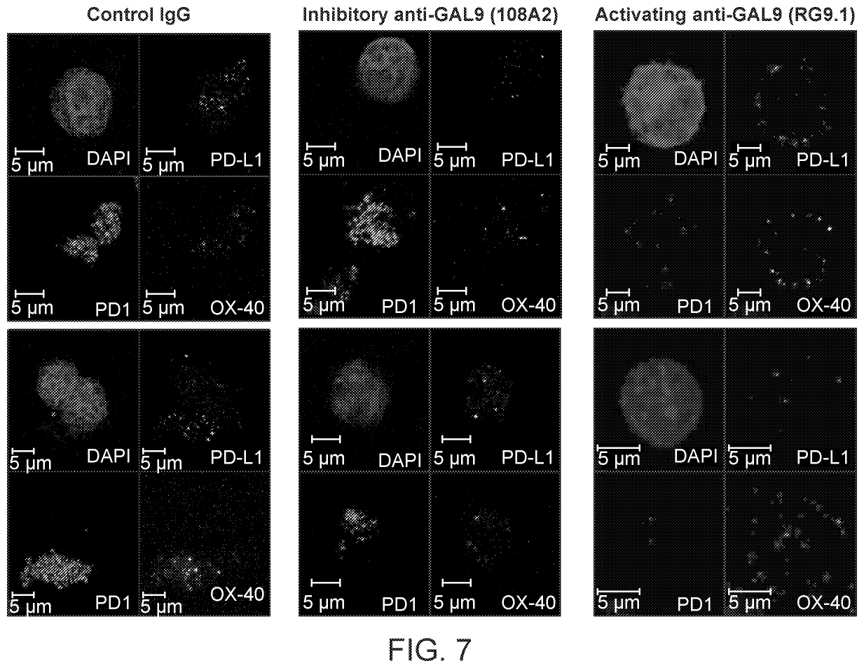

FIG. 7 shows representative confocal images of CD4⁺ T cells treated with either IgG control, inhibitory anti-mouse GAL9 antibody (108A2), or the activating anti-mouse GAL9 antibody (RG9.1). The red staining shows the PD-1 receptor, the green staining shows the PD-L1 ligand, the yellow staining shows the OX40 receptor, and the blue staining shows DNA (DAPI), rendered in gray scale in the attached figures.

We observed that treatment with the activating anti-mouse GAL9 (RG9.1) antibody reduces the expression of PD-1 receptor (low levels of staining) and the PD-L1 ligand (very reduced levels of staining). In contrast, we observed that treatment with inhibitory anti-GAL9 (108A2) had no effect on the expression PD-1 receptor (staining levels similar to IgG control levels) or the PD-L1 ligand (staining levels similar to IgG control levels). In addition, we observed that treatment with inhibitory anti-GAL9 (108A2) resulted in decreased expression of OX40. These results suggest that inhibiting GAL9 antibodies work independently from PD-1/PD-L1 pathway in CD4⁺ T cells.

### 6.11.6. Example 5: Treatment with Inhibitory Anti-Mouse GAL9 (108A2) Decreases PD-L2-Mediated Survival of CD4⁺ and CD8⁺ T Cells from Malaria-Infected Mice

This study was conducted to determine the effect of an inhibitory anti-mouse GAL9 (108A2) antibody on PD-L2-mediated survival of CD4⁺ and CD8⁺ T cells from malaria-infected mice.

PD-L2 has been shown to mediate the survival of CD4⁺ and CD8⁺ T cells in malaria-infected mice, by increasing the numbers of parasite-specific CD4⁺ and CD8⁺ T cells to protect the mice from the lethal malaria infection. See Karunarathne et al. Immunity (2016). Aug. 16; 45(2):333-45), which is incorporated herein by reference in its entirety.

Malaria-Infected Mouse Model

Cohorts of five C57BL/6 mice were infected with non-lethal malaria (P. yoelii 17XNL). After intravenous injection of 10⁵ P. yoelii infected red cells, the mice were incubated for 7 days to allow infection to take place. All animals used in the study were housed and cared for in accordance with the National Health Medical Research Council (NHMRC) Guidelines for Animal Use.

sPD-L2

As a positive control, CD4⁺ and CD8⁺ T cells were treated with soluble PD-L2 “sPD-L2” custom produced by Geneart (Germany).

Cell Isolation, Treatment, and Viability Assay

CD4⁺ and CD8⁺ T cells were isolated from infected mice by FACS using Miltenyi Biotec Inc. (Auburn, Calif.) kits for untouched CD4⁺ and CD8⁺ T cells and then cultured for 36 hours at 37° C. Next, CD4⁺ and CD8⁺ T cells were treated with either 20 mg/ml of sPD-L2 or 20 mg/ml anti-mouse GAL9 (108A2). After treatment, cells were assayed for viability using a viability dye and flow cytometry.

Results/Conclusion

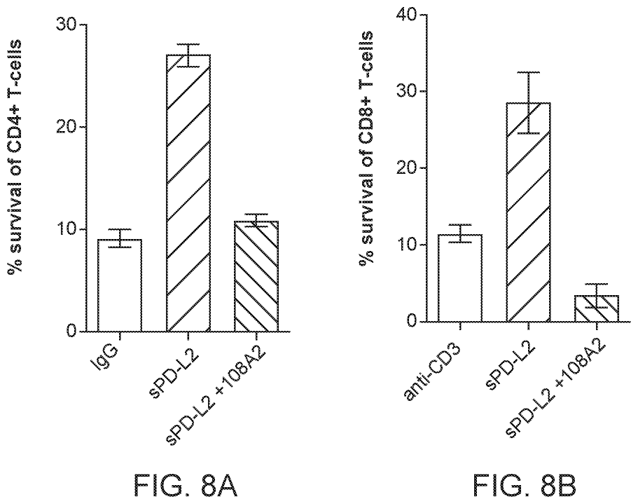

The results for the viability assays for CD4⁺ T cells and CD8⁺ T cell are shown in FIG. 8A and FIG. 8B, respectively. Treatment with sPD-L2 increased PD-L2-mediated survival in CD4⁺ and CD8⁺ T cells. In contrast, treatment with sPD-L2 and anti-GAL9 (108A2) decreased PD-L2-mediated survival in both CD4⁺ and CD8⁺ T cells. These results suggest that PD-L2 works with GAL9 to mediate survival of CD4⁺ and CD8⁺ T cells.

### 6.11.7. Example 6: Blocking the GAL9/PD-L2 Pathway Decreases Proinflammatory Cytokines in Activated CD4⁺ T Cells from Malaria-Infected Mice

This study was conducted to determine if blocking the GAL9/PD-L2 pathway by either a blocking anti-PD-L2 antibody or an inhibitory anti-mouse GAL9 (108A2) antibody can decrease secretion of proinflammatory cytokines in activated CD4⁺ T cells from malaria-infected mice.

Malaria-Infected Mouse Model

Cohorts of five C57BL/6 mice were infected with malaria strain P. yoelii 17XNL and incubated for 7 days, to allow infection to take place. All animals used in the study were housed and cared for in accordance with the NHMRC Guidelines for Animal Use.

Antibodies

The blocking anti-mouse PD-L2 mAb clone TY25 (BioXCell, Lebanon, N.H.) or the inhibitory anti-mouse GAL9 clone 108A2 (BioLegend® San Diego, Calif.) were used.

Cell Isolation and Co-Culture Stimulation

CD4⁺ T cells and DC cells were isolated from malaria-infected mice by using Miltenyi Biotec kits (Auburn, Calif.) for CD4⁺ T cell isolation and CD11c⁺ beads for DC isolation. Next, approximately 1×10⁶ T cells were cultured with 2×10⁵ DCs in at least triplicate wells and then cultured with either 20 ug/ml of anti-PD-L2 mAb or 20 ug/ml of anti-Gal9 mAb for 36 hours.

Cytokine Assays

After treatment, the concentration of INF-γ or TNF-α was measured using BD™ Cytometric Bead Array (CBA) following manufacturer's instructions.

Statistical Analyses

Unpaired t-test with Welch's correction was conducted using GraphPad Prism (GraphPad Software).

Results/Conclusion

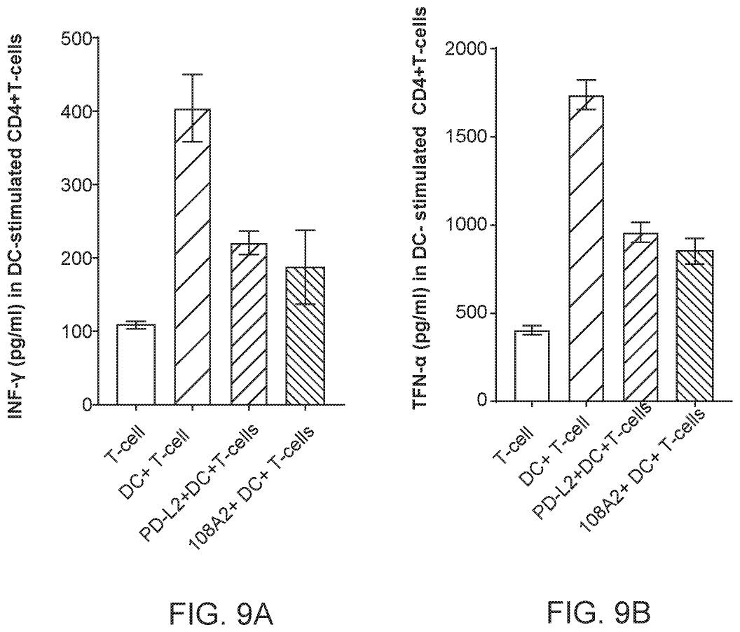

FIG. 9A shows bar graphs of the IFN-γ concentration detected for each treatment group. Treatment with either anti-PD-L2 or anti-GAL9 (108A2) resulted in a significant reduction in IFN-γ levels compared to an untreated co-culture control.

FIG. 9B shows bar graphs of the TNF-α concentration detected for each treatment group. Treatment with either anti-PD-L2 or inhibitory anti-mouse GAL9 antibody (108A2) resulted in a significant reduction of TNF-α levels compared to an untreated co-culture control. The asterisk “*” indicates a statistical significance of p-value <0.05 compared to control. Notably, treatment with anti-PD-L2 and anti-GAL9 (108A2) reduced the IFN-γ and TNF-α to roughly the same concentration level.

### 6.11.8. Example 7: Human GAL9 (Anti-Human GAL9) Binding Arm Discovery Campaign

A chemically synthetic Fab phage library with diversity introduced into the Fab CDRs was screened against GAL9 antigens using a monoclonal phage ELISA format as described above. Phage clones expressing Fabs that recognized GAL9 were sequenced.

The campaign initially identified 52 GAL9 binding candidates (antigen binding site clones). Functional assays conducted after the variable regions of these clones had been reformatted into a bivalent monospecific human IgG1 format identified 30 antibodies having immune inhibiting properties.

Table 3 lists the VH CDR1/2/3 sequences from the 30 inhibiting ABS clones, showing only the residues of the CDRs that had been varied in constructing the library. Table 4 lists the VL CDR1/2/3 sequences from the identified ABS clones; the light chain CDR1 and CDR2 sequences are invariant, and only the residues of CDR3 that were varied in constructing the library are shown.

TABLE 3
Candidate anti-human GAL9 VH Antigen Binding Sites

| | CDR1 | | CDR2 | | CDR3 | |
|---|---|---|---|---|---|---|
| ABS | (variant | SEQ | (variant | SEQ | (variant | SEQ |
| clone | residues) | ID # | residues) | ID # | residues) | ID # |
| P9-01 | SSYW | 7 | WIDPDYGTTS | 59 | AGISYVF | 111 |
| P9-02A | SSYW | 8 | WIDPDYGTTS | 60 | AQYVPGL | 112 |
| P9-03 | SGYY | 10 | VISPYSGYTS | 62 | ATYMVPYGF | 114 |
| P9-06 | AYYG | 13 | YIYPHGYITD | 65 | DSGVPYYWAVL | 117 |
| P9-07 | SSYY | 14 | YISPYGGDTS | 66 | DSYMSYIDGF | 118 |
| P9-11 | SSYY | 18 | YISPSGGYTY | 70 | GAVLYSSAM | 122 |
| P9-12 | SSYW | 19 | SIASYFGQTY | 71 | GFGYAAM | 123 |
| P9-14 | GSYY | 20 | DIYPYFSSTY | 72 | GSHFGF | 124 |
| P9-23 | SQYY | 28 | TIYPRGGYTF | 80 | KSYWGM | 132 |
| P9-24 | SSYF | 29 | SIYPTSHSTS | 81 | LGYPGVM | 133 |
| P9-25 | SSYY | 30 | SIYPYGSYTY | 82 | LGYSSGM | 134 |
| P9-26 | SSYY | 31 | WIESSSSHTD | 83 | LPYKYYYLGVF | 135 |
| P9-29 | SSYA | 34 | YIAPGGSYTY | 86 | LSYPGVM | 138 |
| P9-30 | STYT | 35 | WIYPKGGSTD | 87 | PSGYGF | 139 |
| P9-34 | STYF | 38 | YIYPQGGYTY | 90 | QSYPGVF | 142 |
| P9-37 | WKYG | 40 | YIYPAGGITS | 92 | SDYYSGMGM | 144 |
| P9-38 | SSYW | 41 | WIDPDYGTTS | 93 | SETGAAM | 145 |
| P9-40 | RWYY | 43 | TIYPDWDYTT | 95 | SPVTGPYGF | 147 |
| P9-41 | RYYW | 44 | AIYPSSDSTY | 96 | SSPYPYGQGVF | 148 |
| P9-42 | SSYY | 45 | AIYSAWGTTY | 97 | SYGYVFGYYSGM | 149 |
| P9-43 | HSYW | 46 | RIDSSKFGTY | 98 | SYIDYPVSPAVF | 150 |
| P9-44 | SYYW | 47 | AISPSGSYTS | 99 | SYYRFRTPYTVM | 151 |
| P9-45 | FSYV | 48 | AIYPYSGYTT | 100 | TKYYDYHVF | 152 |
| P9-46 | SRYY | 49 | FISSDSGYTQ | 101 | TMSYSAL | 153 |
| P9-50 | SSYV | 51 | LIYSSGGYTQ | 103 | VGTTYPSRYLEAL | 155 |
| P9-51 | SSYY | 52 | GIYPEGSYTY | 104 | VGYPGVM | 156 |
| P9-52 | STYL | 53 | AITPYSGYTS | 105 | VGYPMVM | 157 |
| P9-53 | SRYQ | 54 | YIASASGTTS | 106 | VPYVAM | 158 |
| P9-56 | SSYY | 56 | YIDSSGKYTD | 108 | YAYPGVM | 160 |
| P9-57 | SSYY | 57 | TIYPSGGYTY | 109 | YSYPGVL | 161 |

TABLE 4
Candidate anti-human GAL9 VL Antigen Binding Sites

| | | | | | CDR3 | |
|---|---|---|---|---|---|---|
| ABS | CDR1 | SEQ | CDR2 | SEQ | (variant | SEQ |
| clone | (Invariant) | ID # | (invariant) | ID # | residues) | ID # |
| P9-01 | RASQSVSSA | 163 | SASSLYS | 215 | QVSDLL | 267 |
| P9-02A | RASQSVSSA | 164 | SASSLYS | 216 | SYPTLG | 268 |
| P9-03 | RASQSVSSA | 166 | SASSLYS | 218 | GGSFPY | 270 |
| P9-06 | RASQSVSSA | 169 | SASSLYS | 221 | HFSSPG | 273 |
| P9-07 | RASQSVSSA | 170 | SASSLYS | 222 | WTSTLW | 274 |
| P9-11 | RASQSVSSA | 174 | SASSLYS | 226 | YYPSPS | 278 |
| P9-12 | RASQSVSSA | 175 | SASSLYS | 227 | EYGRPY | 279 |
| P9-14 | RASQSVSSA | 176 | SASSLYS | 228 | HASGPL | 280 |
| P9-23 | RASQSVSSA | 184 | SASSLYS | 236 | WSVYLE | 288 |
| P9-24 | RASQSVSSA | 185 | SASSLYS | 237 | VDSRLA | 289 |
| P9-25 | RASQSVSSA | 186 | SASSLYS | 238 | WAPDLT | 290 |
| P9-26 | RASQSVSSA | 187 | SASSLYS | 239 | YSSSLY | 291 |
| P9-29 | RASQSVSSA | 190 | SASSLYS | 242 | GYSSLL | 294 |
| P9-30 | RASQSVSSA | 191 | SASSLYS | 243 | YLSSPY | 295 |
| P9-34 | RASQSVSSA | 194 | SASSLYS | 246 | WTIALT | 298 |
| P9-37 | RASQSVSSA | 196 | SASSLYS | 248 | YYPSPS | 300 |
| P9-38 | RASQSVSSA | 197 | SASSLYS | 249 | GSYFLQ | 301 |
| P9-40 | RASQSVSSA | 199 | SASSLYS | 251 | PTYSLW | 303 |
| P9-41 | RASQSVSSA | 200 | SASSLYS | 252 | WYSSLW | 304 |
| P9-42 | RASQSVSSA | 201 | SASSLYS | 253 | WSSDLV | 305 |
| P9-43 | RASQSVSSA | 202 | SASSLYS | 254 | VYFSPY | 306 |
| P9-44 | RASQSVSSA | 203 | SASSLYS | 255 | GIDSPE | 307 |
| P9-45 | RASQSVSSA | 204 | SASSLYS | 256 | GWDSLV | 308 |
| P9-46 | RASQSVSSA | 205 | SASSLYS | 257 | YWWSPE | 309 |
| P9-50 | RASQSVSSA | 207 | SASSLYS | 259 | FGSSLP | 311 |
| P9-51 | RASQSVSSA | 208 | SASSLYS | 260 | WGSSLA | 312 |
| P9-52 | RASQSVSSA | 209 | SASSLYS | 261 | LDYSLA | 313 |
| P9-53 | RASQSVSSA | 210 | SASSLYS | 262 | GYPHPG | 314 |
| P9-56 | RASQSVSSA | 212 | SASSLYS | 264 | YDYSLW | 316 |
| P9-57 | RASQSVSSA | 213 | SASSLYS | 265 | SSSFLW | 317 |

Table 5 presents the full CDR sequences for the human candidate inhibiting anti-GAL9 antibodies according to multiple art-accepted definitions.

TABLE 5
CDR definitions

| | | | | | |
|---|---|---|---|---|---|
| Region | Definition | Sequence | Residues | Length | SEQ ID NO: |

P9-01

| | | | | | |
|---|---|---|---|---|---|
| CDR-H1 | Chothia | GFTFSSY | 26-32 | 7 | 318 |
| | AbM | GFTFSSYWIH | 26-35 | 10 | 319 |
| | Kabat | SYWIH | 31-35 | 5 | 320 |
| | Contact | ----SSYWIH | 30-35 | 6 | 321 |
| | IMGT | GFTFSSYW-- | 26-33 | 8 | 322 |
| CDR-H2 | Chothia | DPDYGT | 52-57 | 6 | 323 |
| | AbM | ---WIDPDYGTTS | 50-59 | 10 | 324 |
| | Kabat | ---WIDPDYGTTSYADSVKG | 50-66 | 17 | 325 |
| | Contact | WVAWIDPDYGTTS | 47-59 | 13 | 326 |
| | IMGT | IDPDYGTT | 51-58 | 8 | 327 |
| CDR-H3 | Chothia | --AGISYVFDY | 99-107 | 9 | 328 |
| | AbM | --AGISYVFDY | 99-107 | 9 | 329 |
| | Kabat | --AGISYVFDY | 99-107 | 9 | 330 |
| | Contact | ARAGISYVFD- | 97-106 | 10 | 331 |
| | IMGT | ARAGISYVFDY | 97-107 | 11 | 332 |
| CDR-L1 | Chothia | RASQSVSSAVA-- | 24-34 | 11 | 333 |
| | AbM | RASQSVSSAVA-- | 24-34 | 11 | 334 |
| | Kabat | RASQSVSSAVA-- | 24-34 | 11 | 335 |
| | Contact | SSAVAWY | 30-36 | 7 | 336 |
| | IMGT | QSVSSA | 27-32 | 6 | 337 |
| CDR-L2 | Chothia | SASSLYS | 50-56 | 7 | 338 |
| | AbM | SASSLYS | 50-56 | 7 | 339 |
| | Kabat | SASSLYS | 50-56 | 7 | 340 |
| | Contact | LLIYSASSLY- | 46-55 | 10 | 341 |
| | IMGT | SA | 50-51 | 2 | 342 |
| CDR-L3 | Chothia | QQQVSDLLT | 89-97 | 9 | 343 |
| | AbM | QQQVSDLLT | 89-97 | 9 | 344 |
| | Kabat | QQQVSDLLT | 89-97 | 9 | 345 |
| | Contact | QQQVSDLL- | 89-96 | 8 | 346 |
| | IMGT | QQQVSDLLT | 89-97 | 9 | 347 |

P9-02A

| | | | | | |
|---|---|---|---|---|---|
| CDR-H1 | Chothia | GFTFSSY--- | 26-32 | 7 | 348 |
| | AbM | GFTFSSYWIH | 26-35 | 10 | 349 |
| | Kabat | SYWIH | 31-35 | 5 | 350 |
| | Contact | SSYWIH | 30-35 | 6 | 351 |
| | IMGT | GFTFSSYW-- | 26-33 | 8 | 352 |
| CDR-H2 | Chothia | DPDYGT | 52-57 | 6 | 353 |
| | AbM | ---WIDPDYGTTS | 50-59 | 10 | 354 |
| | Kabat | ---WIDPDYGTTSYADSVKG | 50-66 | 17 | 355 |
| | Contact | WVAWIDPDYGTTS | 47-59 | 13 | 356 |
| | IMGT | IDPDYGTT | 51-58 | 8 | 357 |
| CDR-H3 | Chothia | --AQYVPGLDY | 99-107 | 9 | 358 |
| | AbM | --AQYVPGLDY | 99-107 | 9 | 359 |
| | Kabat | --AQYVPGLDY | 99-107 | 9 | 360 |
| | Contact | ARAQYVPGLD- | 97-106 | 10 | 361 |
| | IMGT | ARAQYVPGLDY | 97-107 | 11 | 362 |
| CDR-L1 | Chothia | RASQSVSSAVA-- | 24-34 | 11 | 363 |
| | AbM | RASQSVSSAVA-- | 24-34 | 11 | 364 |
| | Kabat | RASQSVSSAVA-- | 24-34 | 11 | 365 |
| | Contact | SSAVAWY | 30-36 | 7 | 366 |
| | IMGT | QSVSSA | 27-32 | 6 | 367 |
| CDR-L2 | Chothia | SASSLYS | 50-56 | 7 | 368 |
| | AbM | ----SASSLYS | 50-56 | 7 | 369 |
| | Kabat | ----SASSLYS | 50-56 | 7 | 370 |
| | Contact | LLTYSASSLY- | 46-55 | 10 | 371 |
| | IMGT | SA | 50-51 | 2 | 372 |
| CDR-L3 | Chothia | QQSYPTLGT | 89-97 | 9 | 373 |
| | AbM | QQSYPTLGT | 89-97 | 9 | 374 |
| | Kabat | QQSYPTLGT | 89-97 | 9 | 375 |
| | Contact | QQSYPTLG- | 89-96 | 8 | 376 |
| | IMGT | QQSYPTLGT | 89-97 | 9 | 377 |

P9-03

| | | | | | |
|---|---|---|---|---|---|
| CDR-H1 | Chothia | GFTFSGY | 26-32 | 7 | 378 |
| | AbM | GFTFSGYYIH | 26-35 | 10 | 379 |
| | Kabat | GYYIH | 31-35 | 5 | 380 |
| | Contact | SGYYIH | 30-35 | 6 | 381 |
| | IMGT | GFTFSGYY-- | 26-33 | 8 | 382 |
| CDR-H2 | Chothia | SPYSGY | 52-57 | 6 | 383 |
| | AbM | ---VISPYSGYTS | 50-59 | 10 | 384 |
| | Kabat | ---VISPYSGYTSYADSVKG | 50-66 | 17 | 385 |
| | Contact | WVAVISPYSGYTS | 47-59 | 13 | 386 |
| | IMGT | ----ISPYSGYT | 51-58 | 8 | 387 |
| CDR-H3 | Chothia | --ATYMVPYGFDY | 99-109 | 11 | 388 |
| | AbM | --ATYMVPYGFDY | 99-109 | 11 | 389 |
| | Kabat | --ATYMVPYGFDY | 99-109 | 11 | 390 |
| | Contact | ARATYMVPYGFD- | 97-108 | 12 | 391 |
| | IMGT | ARATYMVPYGFDY | 97-109 | 13 | 392 |
| CDR-L1 | Chothia | RASQSVSSAVA-- | 24-34 | 11 | 393 |
| | AbM | RASQSVSSAVA-- | 24-34 | 11 | 394 |
| | Kabat | RASQSVSSAVA-- | 24-34 | 11 | 395 |
| | Contact | SSAVAWY | 30-36 | 7 | 396 |
| | IMGT | ---QSVSSA---- | 27-32 | 6 | 397 |
| CDR-L2 | Chothia | SASSLYS | 50-56 | 7 | 398 |
| | AbM | SASSLYS | 50-56 | 7 | 399 |
| | Kabat | SASSLYS | 50-56 | 7 | 400 |
| | Contact | LLIYSASSLY- | 46-55 | 10 | 401 |
| | IMGT | ----SA | 50-51 | 2 | 402 |
| CDR-L3 | Chothia | QQGGSFPYT | 89-97 | 9 | 403 |
| | AbM | QQGGSFPYT | 89-97 | 9 | 404 |
| | Kabat | QQGGSFPYT | 89-97 | 9 | 405 |
| | Contact | QQGGSFPY- | 89-96 | 8 | 406 |
| | IMGT | QQGGSFPYT | 89-97 | 9 | 407 |

P9-06

| | | | | | |
|---|---|---|---|---|---|
| CDR-H1 | Chothia | GFTFAYY | 26-32 | 7 | 408 |
| | AbM | GFTFAYYGIH | 26-35 | 10 | 409 |
| | Kabat | YYGIH | 31-35 | 5 | 410 |
| | Contact | AYYGIH | 30-35 | 6 | 411 |
| | IMGT | GFTFAYYG-- | 26-33 | 8 | 412 |
| CDR-H2 | Chothia | YPHCYI | 52-57 | 6 | 413 |
| | AbM | ---YIYPHGYITD | 50-59 | 10 | 414 |
| | Kabat | ---YIYPHGYITDYADSVKG | 50-66 | 17 | 415 |
| | Contact | WVAYIYPHGYITD | 47-59 | 13 | 416 |
| | IMGT | IYPHGYIT | 51-58 | 8 | 417 |
| CDR-H3 | Chothia | --DSGVPYYWAVLDY | 99-111 | 13 | 418 |
| | AbM | --DSGVPYYWAVLDY | 99-111 | 13 | 419 |
| | Kabat | --DSGVPYYWAVLDY | 99-111 | 13 | 420 |
| | Contact | ARDSGVPYYWAVLD- | 97-110 | 14 | 421 |
| | IMGT | ARDSGVPYYWAVLDY | 97-111 | 15 | 422 |
| CDR-L1 | Chothia | RASQSVSSAVA-- | 24-34 | 11 | 423 |
| | AbM | RASQSVSSAVA-- | 24-34 | 11 | 424 |
| | Kabat | RASQSVSSAVA-- | 24-34 | 11 | 425 |
| | Contact | SSAVAWY | 30-36 | 7 | 426 |
| | IMGT | QSVSSA | 27-32 | 6 | 427 |
| CDR-L2 | Chothia | ----SASSLYS | 50-56 | 7 | 428 |
| | AbM | ----SASSLYS | 50-56 | 7 | 429 |
| | Kabat | SASSLYS | 50-56 | 7 | 430 |
| | Contact | LLIYSASSLY- | 46-55 | 10 | 431 |
| | IMGT | SA | 50-51 | 2 | 432 |
| CDR-L3 | Chothia | QQHFSSPGT | 89-97 | 9 | 433 |
| | AbM | QQHFSSPGT | 89-97 | 9 | 434 |
| | Kabat | QQHFSSPGT | 89-97 | 9 | 435 |
| | Contact | QQHFSSPG- | 89-96 | 8 | 436 |
| | IMGT | QQHFSSPGT | 89-97 | 9 | 437 |

P9-07

| | | | | | |
|---|---|---|---|---|---|
| CDR-H1 | Chothia | GFTFSSY | 26-32 | 7 | 438 |
| | AbM | GFTFSSYYIH | 26-35 | 10 | 439 |
| | Kabat | SYYIH | 31-35 | 5 | 440 |
| | Contact | SSYYIH | 30-35 | 6 | 441 |
| | IMGT | GFTFSSYY-- | 26-33 | 8 | 442 |
| CDR-H2 | Chothia | SPYGGD | 52-57 | 6 | 443 |
| | AbM | ---YISPYGGDTS | 50-59 | 10 | 444 |
| | Kabat | ---YISPYGGDTSYADSVKG | 50-66 | 17 | 445 |
| | Contact | WVAYISPYGGDTS | 47-59 | 13 | 446 |
| | IMGT | ----ISPYGGDT | 51-58 | 8 | 447 |
| CDR-H3 | Chothia | --DSYMSYIDGFDY | 99-110 | 12 | 448 |
| | AbM | --DSYMSYIDGFDY | 99-110 | 12 | 449 |
| | Kabat | --DSYMSYIDGFDY | 99-110 | 12 | 450 |
| | Contact | ARDSYMSYIDGFD- | 97-109 | 13 | 451 |
| | IMGT | ARDSYMSYIDGFDY | 97-110 | 14 | 452 |
| CDR-L1 | Chothia | RASQSVSSAVA-- | 24-34 | 11 | 453 |
| | AbM | RASQSVSSAVA-- | 24-34 | 11 | 454 |
| | Kabat | RASQSVSSAVA-- | 24-34 | 11 | 455 |
| | Contact | SSAVAWY | 30-36 | 7 | 456 |
| | IMGT | ---QSVSSA---- | 27-32 | 6 | 457 |
| CDR-L2 | Chothia | SASSLYS | 50-56 | 7 | 458 |
| | AbM | SASSLYS | 50-56 | 7 | 459 |
| | Kabat | SASSLYS | 50-56 | 7 | 460 |
| | Contact | LLIYSASSLY- | 46-55 | 10 | 461 |
| | IMGT | ----SA | 50-51 | 2 | 462 |
| CDR-L3 | Chothia | QQWTSTLWT | 89-97 | 9 | 463 |
| | AbM | QQWTSTLWT | 89-97 | 9 | 464 |
| | Kabat | QQWTSTLWT | 89-97 | 9 | 465 |
| | Contact | QQWTSTLW- | 89-96 | 8 | 466 |
| | IMGT | QQWTSTLWT | 89-97 | 9 | 467 |

P9-11

| | | | | | |
|---|---|---|---|---|---|
| CDR-H1 | Chothia | GFTFSSY | 26-32 | 7 | 468 |
| | AbM | GFTFSSYYIH | 26-35 | 10 | 469 |
| | Kabat | SYYIH | 31-35 | 5 | 470 |
| | Contact | SSYYIH | 30-35 | 6 | 471 |
| | IMGT | GFTFSSYY-- | 26-33 | 8 | 472 |
| CDR-H2 | Chothia | SPSGGY | 52-57 | 6 | 473 |
| | AbM | ---YISPSGGYTY | 50-59 | 10 | 474 |
| | Kabat | ---YISPSGGYTYYADSVKG | 50-66 | 17 | 475 |
| | Contact | WVAYISPSGGYTY | 47-59 | 13 | 476 |
| | IMGT | ----ISPSGGYT | 51-58 | 8 | 477 |
| CDR-H3 | Chothia | --GAVLYSSAMDY | 99-109 | 11 | 478 |
| | AbM | --GAVLYSSAMDY | 99-109 | 11 | 479 |
| | Kabat | --GAVLYSSAMDY | 99-109 | 11 | 480 |
| | Contact | ARGAVLYSSAMD- | 97-108 | 12 | 481 |
| | IMGT | ARGAVLYSSAMDY | 97-109 | 13 | 482 |
| CDR-L1 | Chothia | RASQSVSSAVA-- | 24-34 | 11 | 483 |
| | AbM | RASQSVSSAVA-- | 24-34 | 11 | 484 |
| | Kabat | RASQSVSSAVA-- | 24-34 | 11 | 485 |
| | Contact | SSAVAWY | 30-36 | 7 | 486 |
| | IMGT | QSVSSA | 27-32 | 6 | 487 |
| CDR-L2 | Chothia | SASSLYS | 50-56 | 7 | 488 |
| | AbM | SASSLYS | 50-56 | 7 | 489 |
| | Kabat | SASSLYS | 50-56 | 7 | 490 |
| | Contact | LLIYSASSLY- | 46-55 | 10 | 491 |
| | IMGT | ----SA | 50-51 | 2 | 492 |
| CDR-L3 | Chothia | QQYYPSPST | 89-97 | 9 | 493 |
| | AbM | QQYYPSPST | 89-97 | 9 | 494 |
| | Kabat | QQYYPSPST | 89-97 | 9 | 495 |
| | Contact | QQYYPSPS- | 89-96 | 8 | 496 |
| | IMGT | QQYYPSPST | 89-97 | 9 | 497 |

P9-12

| | | | | | |
|---|---|---|---|---|---|
| CDR-H1 | Chothia | GFTFSSY--- | 26-32 | 7 | 498 |
| | AbM | GFTFSSYWTH | 26-35 | 10 | 499 |
| | Kabat | SYWTH | 31-35 | 5 | 500 |
| | Contact | SSYWIH | 30-35 | 6 | 501 |
| | IMGT | GFTFSSYW-- | 26-33 | 8 | 502 |
| CDR-H2 | Chothia | ASYFGQ | 52-57 | 6 | 503 |
| | AbM | ---SIASYFGQTY | 50-59 | 10 | 504 |
| | Kabat | ---SIASYFGQTYYADSVKG | 50-66 | 17 | 505 |
| | Contact | WVASIASYFGQTY | 47-59 | 13 | 506 |
| | IMGT | ----IASYFGQT | 51-58 | 8 | 507 |
| CDR-H3 | Chothia | --GFGYAAMDY | 99-107 | 9 | 508 |
| | AbM | --GFGYAAMDY | 99-107 | 9 | 509 |
| | Kabat | --GFGYAAMDY | 99-107 | 9 | 510 |
| | Contact | ARGFGYAAMD- | 97-106 | 10 | 511 |
| | IMGT | ARGFGYAAMDY | 97-107 | 11 | 512 |
| CDR-L1 | Chothia | RASQSVSSAVA-- | 24-34 | 11 | 513 |
| | AbM | RASQSVSSAVA-- | 24-34 | 11 | 514 |
| | Kabat | RASQSVSSAVA-- | 24-34 | 11 | 515 |
| | Contact | SSAVAWY | 30-36 | 7 | 516 |
| | IMGT | QSVSSA | 27-32 | 6 | 517 |
| CDR-L2 | Chothia | SASSLYS | 50-56 | 7 | 518 |
| | AbM | SASSLYS | 50-56 | 7 | 519 |
| | Kabat | SASSLYS | 50-56 | 7 | 520 |
| | Contact | LLIYSASSLY- | 46-55 | 10 | 521 |
| | IMGT | SA | 50-51 | 2 | 522 |
| CDR-L3 | Chothia | QQEYGRPYT | 89-97 | 9 | 523 |
| | AbM | QQEYGRPYT | 89-97 | 9 | 524 |
| | Kabat | QQEYGRPYT | 89-97 | 9 | 525 |
| | Contact | QQEYGRPY- | 89-96 | 8 | 526 |
| | IMGT | QQEYGRPYT | 89-97 | 9 | 527 |

P9-14

| | | | | | |
|---|---|---|---|---|---|
| CDR-H1 | Chothia | GFTFGSY | 26-32 | 7 | 528 |
| | AbM | GFTFGSYYIH | 26-35 | 10 | 529 |
| | Kabat | SYYIH | 31-35 | 5 | 530 |
| | Contact | ----GSYYIH | 30-35 | 6 | 531 |
| | IMGT | GFTFGSYY-- | 26-33 | 8 | 532 |
| CDR-H2 | Chothia | YPYFSS | 52-57 | 6 | 533 |
| | AbM | ---DIYPYFSSTY | 50-59 | 10 | 534 |
| | Kabat | ---DIYPYFSSTYYADSVKG | 50-66 | 17 | 535 |
| | Contact | WVADIYPYFSSTY | 47-59 | 13 | 536 |
| | IMGT | ----IYPYFSST | 51-58 | 8 | 537 |
| CDR-H3 | Chothia | --GSHFGFDY | 99-106 | 8 | 538 |
| | AbM | --GSHFGFDY | 99-106 | 8 | 539 |
| | Kabat | --GSHFGFDY | 99-106 | 8 | 540 |
| | Contact | ARGSHFGFD- | 97-105 | 9 | 541 |
| | IMGT | ARGSHFGFDY | 97-106 | 10 | 542 |
| CDR-L1 | Chothia | RASQSVSSAVA-- | 24-34 | 11 | 543 |
| | AbM | RASQSVSSAVA-- | 24-34 | 11 | 544 |
| | Kabat | RASQSVSSAVA-- | 24-34 | 11 | 545 |
| | Contact | SSAVAWY | 30-36 | 7 | 546 |
| | IMGT | ---QSVSSA---- | 27-32 | 6 | 547 |
| CDR-L2 | Chothia | ----SASSLYS | 50-56 | 7 | 548 |
| | AbM | ----SASSLYS | 50-56 | 7 | 549 |
| | Kabat | SASSLYS | 50-56 | 7 | 550 |
| | Contact | LLIYSASSLY- | 46-55 | 10 | 551 |
| | IMGT | SA | 50-51 | 2 | 552 |
| CDR-L3 | Chothia | QQHASGPLT | 89-97 | 9 | 553 |
| | AbM | QQHASGPLT | 89-97 | 9 | 554 |
| | Kabat | QQHASGPLT | 89-97 | 9 | 555 |
| | Contact | QQHASGPL- | 89-96 | 8 | 556 |
| | IMGT | QQHASGPLT | 89-97 | 9 | 557 |

P9-23

| | | | | | |
|---|---|---|---|---|---|
| CDR-H1 | Chothia | GFTFSQY--- | 26-32 | 7 | 558 |
| | AbM | GFTFSQYYIH | 26-35 | 10 | 559 |
| | Kabat | QYYIH | 31-35 | 5 | 560 |
| | Contact | SQYYIH | 30-35 | 6 | 561 |
| | IMGT | GFTFSQYY-- | 26-33 | 8 | 562 |
| CDR-H2 | Chothia | YPRGGY | 52-57 | 6 | 563 |
| | AbM | ---TIYPRGGYTF | 50-59 | 10 | 564 |
| | Kabat | ---TIYPRGGYTFYADSVKG | 50-66 | 17 | 565 |
| | Contact | WVATIYPRGGYTF | 47-59 | 13 | 566 |
| | IMGT | IYPRGGYT | 51-58 | 8 | 567 |
| CDR-H3 | Chothia | --KSYWGMDY | 99-106 | 8 | 568 |
| | AbM | --KSYWGMDY | 99-106 | 8 | 569 |
| | Kabat | --KSYWGMDY | 99-106 | 8 | 570 |
| | Contact | ARKSYWGMD- | 97-105 | 9 | 571 |
| | IMGT | ARKSYWGMDY | 97-106 | 10 | 572 |
| CDR-L1 | Chothia | RASQSVSSAVA-- | 24-34 | 11 | 573 |
| | AbM | RASQSVSSAVA-- | 24-34 | 11 | 574 |
| | Kabat | RASQSVSSAVA-- | 24-34 | 11 | 575 |
| | Contact | SSAVAWY | 30-36 | 7 | 576 |
| | IMGT | QSVSSA | 27-32 | 6 | 577 |
| CDR-L2 | Chothia | SASSLYS | 50-56 | 7 | 578 |
| | AbM | SASSLYS | 50-56 | 7 | 579 |
| | Kabat | ----SASSLYS | 50-56 | 7 | 580 |
| | Contact | LLIYSASSLY- | 46-55 | 10 | 581 |
| | IMGT | ----SA | 50-51 | 2 | 582 |
| CDR-L3 | Chothia | QQWSVYLET | 89-97 | 9 | 583 |
| | AbM | QQWSVYLET | 89-97 | 9 | 584 |
| | Kabat | QQWSVYLET | 89-97 | 9 | 585 |
| | Contact | QQWSVYLE- | 89-96 | 8 | 586 |
| | IMGT | QQWSVYLET | 89-97 | 9 | 587 |

P9-24

| | | | | | |
|---|---|---|---|---|---|
| CDR-H1 | Chothia | GFTFSSY--- | 26-32 | 7 | 588 |
| | AbM | GFTFSSYFIH | 26-35 | 10 | 589 |
| | Kabat | SYFIH | 31-35 | 5 | 590 |
| | Contact | SSYFIH | 30-35 | 6 | 591 |
| | IMGT | GFTFSSYF-- | 26-33 | 8 | 592 |
| CDR-H2 | Chothia | YPTSHS | 52-57 | 6 | 593 |
| | AbM | ---SIYPTSHSTS | 50-59 | 10 | 594 |
| | Kabat | ---SIYPTSHSTSYADSVKG | 50-66 | 17 | 595 |
| | Contact | WVASIYPTSHSTS | 47-59 | 13 | 596 |
| | IMGT | IYPTSHST | 51-58 | 8 | 597 |
| CDR-H3 | Chothia | --LGYPGVMDY | 99-107 | 9 | 598 |
| | AbM | --LGYPGVMDY | 99-107 | 9 | 599 |
| | Kabat | --LGYPGVMDY | 99-107 | 9 | 600 |
| | Contact | ARLGYPGVMD- | 97-106 | 10 | 601 |
| | IMGT | ARLGYPGVMDY | 97-107 | 11 | 602 |
| CDR-L1 | Chothia | RASQSVSSAVA-- | 24-34 | 11 | 603 |
| | AbM | RASQSVSSAVA-- | 24-34 | 11 | 604 |
| | Kabat | RASQSVSSAVA-- | 24-34 | 11 | 605 |
| | Contact | SSAVAWY | 30-36 | 7 | 606 |
| | IMGT | ---QSVSSA---- | 27-32 | 6 | 607 |
| CDR-L2 | Chothia | ----SASSLYS | 50-56 | 7 | 608 |
| | AbM | SASSLYS | 50-56 | 7 | 609 |
| | Kabat | SASSLYS | 50-56 | 7 | 610 |
| | Contact | LLIYSASSLY- | 46-55 | 10 | 611 |
| | IMGT | SA | 50-51 | 2 | 612 |
| CDR-L3 | Chothia | QQVDSRLAT | 89-97 | 9 | 613 |
| | AbM | QQVDSRLAT | 89-97 | 9 | 614 |
| | Kabat | QQVDSRLAT | 89-97 | 9 | 615 |
| | Contact | QQVDSRLA- | 89-96 | 8 | 616 |
| | IMGT | QQVDSRLAT | 89-97 | 9 | 617 |

P9-25

| | | | | | |
|---|---|---|---|---|---|
| CDR-H1 | Chothia | GFTFSSY | 26-32 | 7 | 618 |
| | AbM | GFTFSSYYIH | 26-35 | 10 | 619 |
| | Kabat | SYYIH | 31-35 | 5 | 620 |
| | Contact | SSYYIH | 30-35 | 6 | 621 |
| | IMGT | GFTFSSYY-- | 26-33 | 8 | 622 |
| CDR-H2 | Chothia | YPYGSY | 52-57 | 6 | 623 |
| | AbM | ---SIYPYGSYTY | 50-59 | 10 | 624 |
| | Kabat | ---SIYPYGSYTYYADSVKG | 50-66 | 17 | 625 |
| | Contact | WVASIYPYGSYTY | 47-59 | 13 | 626 |
| | IMGT | IYPYGSYT | 51-58 | 8 | 627 |
| CDR-H3 | Chothia | --LGYSSGMDY | 99-107 | 9 | 628 |
| | AbM | --LGYSSGMDY | 99-107 | 9 | 629 |
| | Kabat | --LGYSSGMDY | 99-107 | 9 | 630 |
| | Contact | ARLGYSSGMD- | 97-106 | 10 | 631 |
| | IMGT | ARLGYSSGMDY | 97-107 | 11 | 632 |
| CDR-L1 | Chothia | RASQSVSSAVA-- | 24-34 | 11 | 633 |
| | AbM | RASQSVSSAVA-- | 24-34 | 11 | 634 |
| | Kabat | RASQSVSSAVA-- | 24-34 | 11 | 635 |
| | Contact | SSAVAWY | 30-36 | 7 | 636 |
| | IMGT | QSVSSA | 27-32 | 6 | 637 |
| CDR-L2 | Chothia | SASSLYS | 50-56 | 7 | 638 |
| | AbM | ----SASSLYS | 50-56 | 7 | 639 |
| | Kabat | ----SASSLYS | 50-56 | 7 | 640 |
| | Contact | LLIYSASSLY- | 46-55 | 10 | 641 |
| | IMGT | SA | 50-51 | 2 | 642 |
| CDR-L3 | Chothia | QQWAPDLTT | 89-97 | 9 | 643 |
| | AbM | QQWAPDLTT | 89-97 | 9 | 644 |
| | Kabat | QQWAPDLTT | 89-97 | 9 | 645 |
| | Contact | QQWAPDLT- | 89-96 | 8 | 646 |
| | IMGT | QQWAPDLTT | 89-97 | 9 | 647 |

P9-26

| | | | | | |
|---|---|---|---|---|---|
| CDR-H1 | Chothia | GFTFSSY | 26-32 | 7 | 648 |
| | AbM | GFTFSSYYIH | 26-35 | 10 | 649 |
| | Kabat | SYYIH | 31-35 | 5 | 650 |
| | Contact | SSYYIH | 30-35 | 6 | 651 |
| | IMGT | GFTFSSYY-- | 26-33 | 8 | 652 |
| CDR-H2 | Chothia | ESSSSH | 52-57 | 6 | 653 |
| | AbM | ---WIESSSSHTD | 50-59 | 10 | 654 |
| | Kabat | ---WIESSSSHTDYADSVKG | 50-66 | 17 | 655 |
| | Contact | WVAWIESSSSHTD | 47-59 | 13 | 656 |
| | IMGT | ----IESSSSHT | 51-58 | 8 | 657 |
| CDR-H3 | Chothia | --LPYKYYYLGVFDY | 99-111 | 13 | 658 |
| | AbM | --LPYKYYYLGVFDY | 99-111 | 13 | 659 |
| | Kabat | --LPYKYYYLGVFDY | 99-111 | 13 | 660 |
| | Contact | ARLPYKYYYLGVFD- | 97-110 | 14 | 661 |
| | IMGT | ARLPYKYYYLGVFDY | 97-111 | 15 | 662 |
| CDR-L1 | Chothia | RASQSVSSAVA-- | 24-34 | 11 | 663 |
| | AbM | RASQSVSSAVA-- | 24-34 | 11 | 664 |
| | Kabat | RASQSVSSAVA-- | 24-34 | 11 | 665 |
| | Contact | SSAVAWY | 30-36 | 7 | 666 |
| | IMGT | QSVSSA | 27-32 | 6 | 667 |
| CDR-L2 | Chothia | SASSLYS | 50-56 | 7 | 668 |
| | AbM | SASSLYS | 50-56 | 7 | 669 |
| | Kabat | ----SASSLYS | 50-56 | 7 | 670 |
| | Contact | LLIYSASSLY- | 46-55 | 10 | 671 |
| | IMGT | ----SA | 50-51 | 2 | 672 |
| CDR-L3 | Chothia | QQYSSSLYT | 89-97 | 9 | 673 |
| | AbM | QQYSSSLYT | 89-97 | 9 | 674 |
| | Kabat | QQYSSSLYT | 89-97 | 9 | 675 |
| | Contact | QQYSSSLY- | 89-96 | 8 | 676 |
| | IMGT | QQYSSSLYT | 89-97 | 9 | 677 |

P9-29

| | | | | | |
|---|---|---|---|---|---|
| CDR-H1 | Chothia | GFTFSSY | 26-32 | 7 | 678 |
| | AbM | GFTFSSYAIH | 26-35 | 10 | 679 |
| | Kabat | SYAIH | 31-35 | 5 | 680 |
| | Contact | SSYAIH | 30-35 | 6 | 681 |
| | IMGT | GFTFSSYA-- | 26-33 | 8 | 682 |
| CDR-H2 | Chothia | APGGSY | 52-57 | 6 | 683 |
| | AbM | ---YIAPGGSYTY | 50-59 | 10 | 684 |
| | Kabat | ---YIAPGGSYTYYADSVKG | 50-66 | 17 | 685 |
| | Contact | WVAYIAPGGSYTY | 47-59 | 13 | 686 |
| | IMGT | IAPGGSYT | 51-58 | 8 | 687 |
| CDR-H3 | Chothia | --LSYPGVMDY | 99-107 | 9 | 688 |
| | AbM | --LSYPGVMDY | 99-107 | 9 | 689 |
| | Kabat | --LSYPGVMDY | 99-107 | 9 | 690 |
| | Contact | ARLSYPGVMD- | 97-106 | 10 | 691 |
| | IMGT | ARLSYPGVMDY | 97-107 | 11 | 692 |
| CDR-L1 | Chothia | RASQSVSSAVA-- | 24-34 | 11 | 693 |
| | AbM | RASQSVSSAVA-- | 24-34 | 11 | 694 |
| | Kabat | RASQSVSSAVA-- | 24-34 | 11 | 695 |
| | Contact | SSAVAWY | 30-36 | 7 | 696 |
| | IMGT | ---QSVSSA---- | 27-32 | 6 | 697 |
| CDR-L2 | Chothia | SASSLYS | 50-56 | 7 | 698 |
| | AbM | SASSLYS | 50-56 | 7 | 699 |
| | Kabat | SASSLYS | 50-56 | 7 | 700 |
| | Contact | LLIYSASSLY- | 46-55 | 10 | 701 |
| | IMGT | SA | 50-51 | 2 | 702 |
| CDR-L3 | Chothia | QQGYSSLLT | 89-97 | 9 | 703 |
| | AbM | QQGYSSLLT | 89-97 | 9 | 704 |
| | Kabat | QQGYSSLLT | 89-97 | 9 | 705 |
| | Contact | QQGYSSLL- | 89-96 | 8 | 706 |
| | IMGT | QQGYSSLLT | 89-97 | 9 | 707 |

P9-30

| | | | | | |
|---|---|---|---|---|---|
| CDR-H1 | Chothia | GFTFSTY--- | 26-32 | 7 | 708 |
| | AbM | GFTFSTYTIH | 26-35 | 10 | 709 |
| | Kabat | TYTIH | 31-35 | 5 | 710 |
| | Contact | STYTIH | 30-35 | 6 | 711 |
| | IMGT | GFTFSTYT-- | 26-33 | 8 | 712 |
| CDR-H2 | Chothia | YPKGGS | 52-57 | 6 | 713 |
| | AbM | ---WIYPKGGSTD | 50-59 | 10 | 714 |
| | Kabat | ---WIYPKGGSTDYADSVKG | 50-66 | 17 | 715 |
| | Contact | WVAWIYPKGGSTD | 47-59 | 13 | 716 |
| | IMGT | ----IYPKGGST | 51-58 | 8 | 717 |
| CDR-H3 | Chothia | --PSGYGFDY | 99-106 | 8 | 718 |
| | AbM | --PSGYGFDY | 99-106 | 8 | 719 |
| | Kabat | --PSGYGFDY | 99-106 | 8 | 720 |
| | Contact | ARPSGYGFD- | 97-105 | 9 | 721 |
| | IMGT | ARPSGYGFDY | 97-106 | 10 | 722 |
| CDR-L1 | Chothia | RASQSVSSAVA-- | 24-34 | 11 | 723 |
| | AbM | RASQSVSSAVA-- | 24-34 | 11 | 724 |
| | Kabat | RASQSVSSAVA-- | 24-34 | 11 | 725 |
| | Contact | SSAVAWY | 30-36 | 7 | 726 |
| | IMGT | QSVSSA | 27-32 | 6 | 727 |
| CDR-L2 | Chothia | SASSLYS | 50-56 | 7 | 728 |
| | AbM | SASSLYS | 50-56 | 7 | 729 |
| | Kabat | SASSLYS | 50-56 | 7 | 730 |
| | Contact | LLIYSASSLY- | 46-55 | 10 | 731 |
| | IMGT | ----SA | 50-51 | 2 | 732 |
| CDR-L3 | Chothia | QQYLSSPYT | 89-97 | 9 | 733 |
| | AbM | QQYLSSPYT | 89-97 | 9 | 734 |
| | Kabat | QQYLSSPYT | 89-97 | 9 | 735 |
| | Contact | QQYLSSPY- | 89-96 | 8 | 736 |
| | IMGT | QQYLSSPYT | 89-97 | 9 | 737 |

P9-34

| | | | | | |
|---|---|---|---|---|---|
| CDR-H1 | Chothia | GFTFSTY | 26-32 | 7 | 738 |
| | AbM | GFTFSTYFIH | 26-35 | 10 | 739 |
| | Kabat | TYFIH | 31-35 | 5 | 740 |
| | Contact | ----STYFIH | 30-35 | 6 | 741 |
| | IMGT | GFTFSTYF-- | 26-33 | 8 | 742 |
| CDR-H2 | Chothia | YPQGGY | 52-57 | 6 | 743 |
| | AbM | ---YIYPQGGYTY | 50-59 | 10 | 744 |
| | Kabat | ---YIYPQGGYTYYADSVKG | 50-66 | 17 | 745 |
| | Contact | WVAYIYPQGGYTY | 47-59 | 13 | 746 |
| | IMGT | IYPQGGYT | 51-58 | 8 | 747 |
| CDR-H3 | Chothia | --QSYPGVFDY | 99-107 | 9 | 748 |
| | AbM | --QSYPCVFDY | 99-107 | 9 | 749 |
| | Kabat | --QSYPGVFDY | 99-107 | 9 | 750 |
| | Contact | ARQSYPGVFD- | 97-106 | 10 | 751 |
| | IMGT | ARQSYPGVFDY | 97-107 | 11 | 752 |
| CDR-L1 | Chothia | RASQSVSSAVA-- | 24-34 | 11 | 753 |
| | AbM | RASQSVSSAVA-- | 24-34 | 11 | 754 |
| | Kabat | RASQSVSSAVA-- | 24-34 | 11 | 755 |
| | Contact | SSAVAWY | 30-36 | 7 | 756 |
| | IMGT | ---QSVSSA---- | 27-32 | 6 | 757 |
| CDR-L2 | Chothia | ----SASSLYS | 50-56 | 7 | 758 |
| | AbM | ----SASSLYS | 50-56 | 7 | 759 |
| | Kabat | SASSLYS | 50-56 | 7 | 760 |
| | Contact | LLIYSASSLY- | 46-55 | 10 | 761 |
| | IMGT | SA | 50-51 | 2 | 762 |
| CDR-L3 | Chothia | QQWTIALTT | 89-97 | 9 | 763 |
| | AbM | QQWTIALTT | 89-97 | 9 | 764 |
| | Kabat | QQWTIALTT | 89-97 | 9 | 765 |
| | Contact | QQWTIALT- | 89-96 | 8 | 766 |
| | IMGT | QQWTIALTT | 89-97 | 9 | 767 |

P9-37

| | | | | | |
|---|---|---|---|---|---|
| CDR-H1 | Chothia | GFTFSSY--- | 26-32 | 7 | 768 |
| | AbM | GFTFSSYWIH | 26-35 | 10 | 769 |
| | Kabat | SYWIH | 31-35 | 5 | 770 |
| | Contact | SSYWIH | 30-35 | 6 | 771 |
| | IMGT | GFTFSSYW-- | 26-33 | 8 | 772 |
| CDR-H2 | Chothia | DPDYGT | 52-57 | 6 | 773 |
| | AbM | ---WIDPDYGTTS | 50-59 | 10 | 774 |
| | Kabat | ---WIDPDYGTTSYADSVKG | 50-66 | 17 | 775 |
| | Contact | WVAWIDPDYGTTS | 47-59 | 13 | 776 |
| | IMGT | IDPDYGTT | 51-58 | 8 | 777 |
| CDR-H3 | Chothia | --SETGAAMDY | 99-107 | 9 | 778 |
| | AbM | --SETGAAMDY | 99-107 | 9 | 779 |
| | Kabat | --SETGAAMDY | 99-107 | 9 | 780 |
| | Contact | ARSETGAAMD- | 97-106 | 10 | 781 |
| | IMGT | ARSETGAAMDY | 97-107 | 11 | 782 |
| CDR-L1 | Chothia | RASQSVSSAVA-- | 24-34 | 11 | 783 |
| | AbM | RASQSVSSAVA-- | 24-34 | 11 | 784 |
| | Kabat | RASQSVSSAVA-- | 24-34 | 11 | 785 |
| | Contact | SSAVAWY | 30-36 | 7 | 786 |
| | IMGT | QSVSSA | 27-32 | 6 | 787 |
| CDR-L2 | Chothia | SASSLYS | 50-56 | 7 | 788 |
| | AbM | SASSLYS | 50-56 | 7 | 789 |
| | Kabat | ----SASSLYS | 50-56 | 7 | 790 |
| | Contact | LLIYSASSLY- | 46-55 | 10 | 791 |
| | IMGT | ----SA | 50-51 | 2 | 792 |
| CDR-L3 | Chothia | QQGSYFLQT | 89-97 | 9 | 793 |
| | AbM | QQGSYFLQT | 89-97 | 9 | 794 |
| | Kabat | QQGSYFLQT | 89-97 | 9 | 795 |
| | Contact | QQGSYFLQ- | 89-96 | 8 | 796 |
| | IMGT | QQGSYFLQT | 89-97 | 9 | 797 |

P9-40

| | | | | | |
|---|---|---|---|---|---|
| CDR-H1 | Chothia | GFTFRWY | 26-32 | 7 | 798 |
| | AbM | GFTFRWYYIH | 26-35 | 10 | 799 |
| | Kabat | WYYIH | 31-35 | 5 | 800 |
| | Contact | ----RWYYIH | 30-35 | 6 | 801 |
| | IMGT | GFTFRWYY-- | 26-33 | 8 | 802 |
| CDR-H2 | Chothia | YPDWDY | 52-57 | 6 | 803 |
| | AbM | ---TIYPDWDYTT | 50-59 | 10 | 804 |
| | Kabat | ---TIYPDWDYTTYADSVKG | 50-66 | 17 | 805 |
| | Contact | WVATIYPDWDYTT | 47-59 | 13 | 806 |
| | IMGT | IYPDWDYT | 51-58 | 8 | 807 |
| CDR-H3 | Chothia | --SPVTGPYGFDY | 99-109 | 11 | 808 |
| | AbM | --SPVTGPYGFDY | 99-109 | 11 | 809 |
| | Kabat | --SPVTGPYGFDY | 99-109 | 11 | 810 |
| | Contact | ARSPVTGPYGFD- | 97-108 | 12 | 811 |
| | IMGT | ARSPVTGPYGFDY | 97-109 | 13 | 812 |
| CDR-L1 | Chothia | RASQSVSSAVA-- | 24-34 | 11 | 813 |
| | AbM | RASQSVSSAVA-- | 24-34 | 11 | 814 |
| | Kabat | RASQSVSSAVA-- | 24-34 | 11 | 815 |
| | Contact | SSAVAWY | 30-36 | 7 | 816 |
| | IMGT | ---QSVSSA---- | 27-32 | 6 | 817 |
| CDR-L2 | Chothia | ----SASSLYS | 50-56 | 7 | 818 |
| | AbM | ----SASSLYS | 50-56 | 7 | 819 |
| | Kabat | SASSLYS | 50-56 | 7 | 820 |
| | Contact | LLIYSASSLY- | 46-55 | 10 | 821 |
| | IMGT | SA | 50-51 | 2 | 822 |
| CDR-L3 | Chothia | QQPTYSLWT | 89-97 | 9 | 823 |
| | AbM | QQPTYSLWT | 89-97 | 9 | 824 |
| | Kabat | QQPTYSLWT | 89-97 | 9 | 825 |
| | Contact | QQPTYSLW- | 89-96 | 8 | 826 |
| | IMGT | QQPTYSLWT | 89-97 | 9 | 827 |

P9-41

| | | | | | |
|---|---|---|---|---|---|
| CDR-H1 | Chothia | GFTFRYY | 26-32 | 7 | 828 |
| | AbM | GFTFRYYWIH | 26-35 | 10 | 829 |
| | Kabat | YYWIH | 31-35 | 5 | 830 |
| | Contact | RYYWIH | 30-35 | 6 | 831 |
| | IMGT | GFTFRYYW-- | 26-33 | 8 | 832 |
| CDR-H2 | Chothia | YPSSDS | 52-57 | 6 | 833 |
| | AbM | ---AIYPSSDSTY | 50-59 | 10 | 834 |
| | Kabat | ---AIYPSSDSTYYADSVKG | 50-66 | 17 | 835 |
| | Contact | WVAAIYPSSDSTY | 47-59 | 13 | 836 |
| | IMGT | ----IYPSSDST | 51-58 | 8 | 837 |
| CDR-H3 | Chothia | --SSPYPYGQGVFDY | 99-111 | 13 | 838 |
| | AbM | --SSPYPYGQGVFDY | 99-111 | 13 | 839 |
| | Kabat | --SSPYPYGQGVFDY | 99-111 | 13 | 840 |
| | Contact | ARSSPYPYGQGVFD- | 97-110 | 14 | 841 |
| | IMGT | ARSSPYPYGQGVFDY | 97-111 | 15 | 842 |
| CDR-L1 | Chothia | RASQSVSSAVA-- | 24-34 | 11 | 843 |
| | AbM | RASQSVSSAVA-- | 24-34 | 11 | 844 |
| | Kabat | RASQSVSSAVA-- | 24-34 | 11 | 845 |
| | Contact | SSAVAWY | 30-36 | 7 | 846 |
| | IMGT | QSVSSA | 27-32 | 6 | 847 |
| CDR-L2 | Chothia | SASSLYS | 50-56 | 7 | 848 |
| | AbM | SASSLYS | 50-56 | 7 | 849 |
| | Kabat | ----SASSLYS | 50-56 | 7 | 850 |
| | Contact | LLIYSASSLY- | 46-55 | 10 | 851 |
| | IMGT | ----SA | 50-51 | 2 | 852 |
| CDR-L3 | Chothia | QQWYSSLWT | 89-97 | 9 | 853 |
| | AbM | QQWYSSLWT | 89-97 | 9 | 854 |
| | Kabat | QQWYSSLWT | 89-97 | 9 | 855 |
| | Contact | QQWYSSLW- | 89-96 | 8 | 856 |
| | IMGT | QQWYSSLWT | 89-97 | 9 | 857 |

P9-42

| | | | | | |
|---|---|---|---|---|---|
| CDR-H1 | Chothia | GFTFSSY | 26-32 | 7 | 858 |
| | AbM | GFTFSSYYIH | 26-35 | 10 | 859 |
| | Kabat | SYYIH | 31-35 | 5 | 860 |
| | Contact | ----SSYYTH | 30-35 | 6 | 861 |
| | IMGT | GFTFSSYY-- | 26-33 | 8 | 862 |
| CDR-H2 | Chothia | YSAWGT | 52-57 | 6 | 863 |
| | AbM | ---AIYSAWGTTY | 50-59 | 10 | 864 |
| | Kabat | ---AIYSAWGTTYYADSVKG | 50-66 | 17 | 865 |
| | Contact | WVAAIYSAWGTTY | 47-59 | 13 | 866 |
| | IMGT | ----IYSAWGTT | 51-58 | 8 | 867 |
| CDR-H3 | Chothia | --SYGYVFGYYSGMDY | 99-112 | 14 | 868 |
| | AbM | --SYGYVFGYYSGMDY | 99-112 | 14 | 869 |
| | Kabat | --SYGYVFGYYSGMDY | 99-112 | 14 | 870 |
| | Contact | ARSYGYVFGYYSGMD- | 97-111 | 15 | 871 |
| | IMGT | ARSYGYVFGYYSGMDY | 97-112 | 16 | 872 |
| CDR-L1 | Chothia | RASQSVSSAVA-- | 24-34 | 11 | 873 |
| | AbM | RASQSVSSAVA-- | 24-34 | 11 | 874 |
| | Kabat | RASQSVSSAVA-- | 24-34 | 11 | 875 |
| | Contact | SSAVAWY | 30-36 | 7 | 876 |
| | IMGT | ---QSVSSA---- | 27-32 | 6 | 877 |
| CDR-L2 | Chothia | SASSLYS | 50-56 | 7 | 878 |
| | AbM | SASSLYS | 50-56 | 7 | 879 |
| | Kabat | SASSLYS | 50-56 | 7 | 880 |
| | Contact | LLIYSASSLY- | 46-55 | 10 | 881 |
| | IMGT | ----SA | 50-51 | 2 | 882 |
| CDR-L3 | Chothia | QQWSSDLVT | 89-97 | 9 | 883 |
| | AbM | QQWSSDLVT | 89-97 | 9 | 884 |
| | Kabat | QQWSSDLVT | 89-97 | 9 | 885 |
| | Contact | QQWSSDLV- | 89-96 | 8 | 886 |
| | IMGT | QQWSSDLVT | 89-97 | 9 | 887 |

P9-43

| | | | | | |
|---|---|---|---|---|---|
| CDR-H1 | Chothia | GFTFHSY--- | 26-32 | 7 | 888 |
| | AbM | GFTFHSYWIH | 26-35 | 10 | 889 |
| | Kabat | SYWIH | 31-35 | 5 | 890 |
| | Contact | HSYWIH | 30-35 | 6 | 891 |
| | IMGT | GFTFHSYW-- | 26-33 | 8 | 892 |
| CDR-H2 | Chothia | DSSKFG | 52-57 | 6 | 893 |
| | AbM | ---RIDSSKFGTY | 50-59 | 10 | 894 |
| | Kabat | ---RIDSSKFGTYYADSVKG | 50-66 | 17 | 895 |
| | Contact | WVARIDSSKFGTY | 47-59 | 13 | 896 |
| | IMGT | IDSSKFGT | 51-58 | 8 | 897 |
| CDR-H3 | Chothia | --SYIDYPVSPAVFDY | 99-112 | 14 | 898 |
| | AbM | --SYIDYPVSPAVFDY | 99-112 | 14 | 899 |
| | Kabat | --SYIDYPVSPAVFDY | 99-112 | 14 | 900 |
| | Contact | ARSYIDYPVSPAVFD- | 97-111 | 15 | 901 |
| | IMGT | ARSYIDYPVSPAVFDY | 97-112 | 16 | 902 |
| CDR-L1 | Chothia | RASQSVSSAVA-- | 24-34 | 11 | 903 |
| | AbM | RASQSVSSAVA-- | 24-34 | 11 | 904 |
| | Kabat | RASQSVSSAVA-- | 24-34 | 11 | 905 |
| | Contact | SSAVAWY | 30-36 | 7 | 906 |
| | IMGT | QSVSSA | 27-32 | 6 | 907 |
| CDR-L2 | Chothia | ----SASSLYS | 50-56 | 7 | 908 |
| | AbM | SASSLYS | 50-56 | 7 | 909 |
| | Kabat | SASSLYS | 50-56 | 7 | 910 |
| | Contact | LLIYSASSLY- | 46-55 | 10 | 911 |
| | IMGT | SA | 50-51 | 2 | 912 |
| CDR-L3 | Chothia | QQVYFSPYT | 89-97 | 9 | 913 |
| | AbM | QQVYFSPYT | 89-97 | 9 | 914 |
| | Kabat | QQVYFSPYT | 89-97 | 9 | 915 |
| | Contact | QQVYFSPY- | 89-96 | 8 | 916 |
| | IMGT | QQVYFSPYT | 89-97 | 9 | 917 |

P9-44

| | | | | | |
|---|---|---|---|---|---|
| CDR-H1 | Chothia | GFTFSYY | 26-32 | 7 | 918 |
| | AbM | GFTFSYYWIH | 26-35 | 10 | 919 |
| | Kabat | YYWIH | 31-35 | 5 | 920 |
| | Contact | ----SYYWIH | 30-35 | 6 | 921 |
| | IMGT | GFTFSYYW-- | 26-33 | 8 | 922 |
| CDR-H2 | Chothia | SPSGSY | 52-57 | 6 | 923 |
| | AbM | ---AISPSGSYTS | 50-59 | 10 | 924 |
| | Kabat | ---AISPSGSYTSYADSVKG | 50-66 | 17 | 925 |
| | Contact | WVAAISPSGSYTS | 47-59 | 13 | 926 |
| | IMGT | ----ISPSGSYT | 51-58 | 8 | 927 |
| CDR-H3 | Chothia | --SYYRFRTPYTVMDY | 99-112 | 14 | 928 |
| | AbM | --SYYRFRTPYTVMDY | 99-112 | 14 | 929 |
| | Kabat | --SYYRFRTPYTVMDY | 99-112 | 14 | 930 |
| | Contact | ARSYYRFRTPYTVMD- | 97-111 | 15 | 931 |
| | IMGT | ARSYYRFRTPYTVMDY | 97-112 | 16 | 932 |
| CDR-L1 | Chothia | RASQSVSSAVA-- | 24-34 | 11 | 933 |
| | AbM | RASQSVSSAVA-- | 24-34 | 11 | 934 |
| | Kabat | RASQSVSSAVA-- | 24-34 | 11 | 935 |
| | Contact | SSAVAWY | 30-36 | 7 | 936 |
| | IMGT | QSVSSA | 27-32 | 6 | 937 |
| CDR-L2 | Chothia | SASSLYS | 50-56 | 7 | 938 |
| | AbM | SASSLYS | 50-56 | 7 | 939 |
| | Kabat | SASSLYS | 50-56 | 7 | 940 |
| | Contact | LLIYSASSLY- | 46-55 | 10 | 941 |
| | IMGT | ----SA | 50-51 | 2 | 942 |
| CDR-L3 | Chothia | QQGIDSPET | 89-97 | 9 | 943 |
| | AbM | QQGIDSPET | 89-97 | 9 | 944 |
| | Kabat | QQGIDSPET | 89-97 | 9 | 945 |
| | Contact | QQGIDSPE- | 89-96 | 8 | 946 |
| | IMGT | QQGIDSPET | 89-97 | 9 | 947 |

P9-45

| | | | | | |
|---|---|---|---|---|---|
| CDR-H1 | Chothia | GFTFFSY--- | 26-32 | 7 | 948 |
| | AbM | GFTFFSYVIH | 26-35 | 10 | 949 |
| | Kabat | SYVIH | 31-35 | 5 | 950 |
| | Contact | FSYVIH | 30-35 | 6 | 951 |
| | IMGT | GFTFFSYV-- | 26-33 | 8 | 952 |
| CDR-H2 | Chothia | YPYSGY | 52-57 | 6 | 953 |
| | AbM | ---AIYPYSGYTT | 50-59 | 10 | 954 |
| | Kabat | ---AIYPYSGYTTYADSVKG | 50-66 | 17 | 955 |
| | Contact | WVAAIYPYSGYTT | 47-59 | 13 | 956 |
| | IMGT | IYPYSGYT | 51-58 | 8 | 957 |
| CDR-H3 | Chothia | --TKYYDYHVFDY | 99-109 | 11 | 958 |
| | AbM | --TKYYDYHVFDY | 99-109 | 11 | 959 |
| | Kabat | --TKYYDYHVFDY | 99-109 | 11 | 960 |
| | Contact | ARTKYYDYHVFD- | 97-108 | 12 | 961 |
| | IMGT | ARTKYYDYHVFDY | 97-109 | 13 | 962 |
| CDR-L1 | Chothia | RASQSVSSAVA-- | 24-34 | 11 | 963 |
| | AbM | RASQSVSSAVA-- | 24-34 | 11 | 964 |
| | Kabat | RASQSVSSAVA-- | 24-34 | 11 | 965 |
| | Contact | SSAVAWY | 30-36 | 7 | 966 |
| | IMGT | QSVSSA | 27-32 | 6 | 967 |
| CDR-L2 | Chothia | ----SASSLYS | 50-56 | 7 | 968 |
| | AbM | ----SASSLYS | 50-56 | 7 | 969 |
| | Kabat | ----SASSLYS | 50-56 | 7 | 970 |
| | Contact | LLIYSASSLY- | 46-55 | 10 | 971 |
| | IMGT | SA | 50-51 | 2 | 972 |
| CDR-L3 | Chothia | QQGWDSLVT | 89-97 | 9 | 973 |
| | AbM | QQGWDSLVT | 89-97 | 9 | 974 |
| | Kabat | QQGWDSLVT | 89-97 | 9 | 975 |
| | Contact | QQGWDSLV- | 89-96 | 8 | 976 |
| | IMGT | QQGWDSLVT | 89-97 | 9 | 977 |

P9-46

| | | | | | |
|---|---|---|---|---|---|
| CDR-H1 | Chothia | GFTFSRY | 26-32 | 7 | 978 |
| | AbM | GFTFSRYYIH | 26-35 | 10 | 979 |
| | Kabat | RYYIH | 31-35 | 5 | 980 |
| | Contact | ----SRYYIH | 30-35 | 6 | 981 |
| | IMGT | GFTFSRYY-- | 26-33 | 8 | 982 |
| CDR-H2 | Chothia | SSDSGY | 52-57 | 6 | 983 |
| | AbM | ---FISSDSGYTQ | 50-59 | 10 | 984 |
| | Kabat | ---FISSDSGYTQYADSVKG | 50-66 | 17 | 985 |
| | Contact | WVAFISSDSGYTQ | 47-59 | 13 | 986 |
| | IMGT | ISSDSGYT | 51-58 | 8 | 987 |
| CDR-H3 | Chothia | --TMSYSALDY | 99-107 | 9 | 988 |
| | AbM | --TMSYSALDY | 99-107 | 9 | 989 |
| | Kabat | --TMSYSALDY | 99-107 | 9 | 990 |
| | Contact | ARTMSYSALD- | 97-106 | 10 | 991 |
| | IMGT | ARTMSYSALDY | 97-107 | 11 | 992 |
| CDR-L1 | Chothia | RASQSVSSAVA-- | 24-34 | 11 | 993 |
| | AbM | RASQSVSSAVA-- | 24-34 | 11 | 994 |
| | Kabat | RASQSVSSAVA-- | 24-34 | 11 | 995 |
| | Contact | SSAVAWY | 30-36 | 7 | 996 |
| | IMGT | QSVSSA | 27-32 | 6 | 997 |
| CDR-L2 | Chothia | SASSLYS | 50-56 | 7 | 998 |
| | AbM | SASSLYS | 50-56 | 7 | 999 |
| | Kabat | ----SASSLYS | 50-56 | 7 | 1000 |
| | Contact | LLIYSASSLY- | 46-55 | 10 | 1001 |
| | IMGT | ----SA | 50-51 | 2 | 1002 |
| CDR-L3 | Chothia | QQYWWSPET | 89-97 | 9 | 1003 |
| | AbM | QQYWWSPET | 89-97 | 9 | 1004 |
| | Kabat | QQYWWSPET | 89-97 | 9 | 1005 |
| | Contact | QQYWWSPE- | 89-96 | 8 | 1006 |
| | IMGT | QQYWWSPET | 89-97 | 9 | 1007 |

P9-50

| | | | | | |
|---|---|---|---|---|---|
| CDR-H1 | Chothia | GFTFSSY | 26-32 | 7 | 1008 |
| | AbM | GFTFSSYVIH | 26-35 | 10 | 1009 |
| | Kabat | SYVIH | 31-35 | 5 | 1010 |
| | Contact | ----SSYVIH | 30-35 | 6 | 1011 |
| | IMGT | GFTFSSYV-- | 26-33 | 8 | 1012 |
| CDR-H2 | Chothia | YSSGGY | 52-57 | 6 | 1013 |
| | AbM | ---LIYSSGGYTQ | 50-59 | 10 | 1014 |
| | Kabat | ---LIYSSGGYTQYADSVKG | 50-66 | 17 | 1015 |
| | Contact | WVALIYSSGGYTQ | 47-59 | 13 | 1016 |
| | IMGT | IYSSGGYT | 51-58 | 8 | 1017 |
| CDR-H3 | Chothia | --VGTTYPSRYLEALDY | 99-113 | 15 | 1018 |
| | AbM | --VGTTYPSRYLEALDY | 99-113 | 15 | 1019 |
| | Kabat | --VGTTYPSRYLEALDY | 99-113 | 15 | 1020 |
| | Contact | ARVGTTYPSRYLEALD- | 97-112 | 16 | 1021 |
| | IMGT | ARVGTTYPSRYLEALDY | 97-113 | 17 | 1022 |
| CDR-L1 | Chothia | RASQSVSSAVA-- | 24-34 | 11 | 1023 |
| | AbM | RASQSVSSAVA-- | 24-34 | 11 | 1024 |
| | Kabat | RASQSVSSAVA-- | 24-34 | 11 | 1025 |
| | Contact | SSAVAWY | 30-36 | 7 | 1026 |
| | IMGT | ---QSVSSA---- | 27-32 | 6 | 1027 |
| CDR-L2 | Chothia | ----SASSLYS | 50-56 | 7 | 1028 |
| | AbM | ----SASSLYS | 50-56 | 7 | 1029 |
| | Kabat | SASSLYS | 50-56 | 7 | 1030 |
| | Contact | LLIYSASSLY- | 46-55 | 10 | 1031 |
| | IMGT | SA | 50-51 | 2 | 1032 |
| CDR-L3 | Chothia | QQFGSSLPT | 89-97 | 9 | 1033 |
| | AbM | QQFGSSLPT | 89-97 | 9 | 1034 |
| | Kabat | QQFGSSLPT | 89-97 | 9 | 1035 |
| | Contact | QQFGSSLP- | 89-96 | 8 | 1036 |
| | IMGT | QQFGSSLPT | 89-97 | 9 | 1037 |

P9-51

| | | | | | |
|---|---|---|---|---|---|
| CDR-H1 | Chothia | GFTFSSY | 26-32 | 7 | 1038 |
| | AbM | GFTFSSYYIH | 26-35 | 10 | 1039 |
| | Kabat | SYYIH | 31-35 | 5 | 1040 |
| | Contact | SSYYIH | 30-35 | 6 | 1041 |
| | IMGT | GFTFSSYY-- | 26-33 | 8 | 1042 |
| CDR-H2 | Chothia | YPEGSY | 52-57 | 6 | 1043 |
| | AbM | ---GIYPEGSYTY | 50-59 | 10 | 1044 |
| | Kabat | ---GIYPEGSYTYYADSVKG | 50-66 | 17 | 1045 |
| | Contact | WVAGIYPEGSYTY | 47-59 | 13 | 1046 |
| | IMGT | IYPEGSYT | 51-58 | 8 | 1047 |
| CDR-H3 | Chothia | --VGYPGVMDY | 99-107 | 9 | 1048 |
| | AbM | --VGYPGVMDY | 99-107 | 9 | 1049 |
| | Kabat | --VGYPGVMDY | 99-107 | 9 | 1050 |
| | Contact | ARVGYPGVMD- | 97-106 | 10 | 1051 |
| | IMGT | ARVGYPGVMDY | 97-107 | 11 | 1052 |
| CDR-L1 | Chothia | RASQSVSSAVA-- | 24-34 | 11 | 1053 |
| | AbM | RASQSVSSAVA-- | 24-34 | 11 | 1054 |
| | Kabat | RASQSVSSAVA-- | 24-34 | 11 | 1055 |
| | Contact | SSAVAWY | 30-36 | 7 | 1056 |
| | IMGT | QSVSSA | 27-32 | 6 | 1057 |
| CDR-L2 | Chothia | SASSLYS | 50-56 | 7 | 1058 |
| | AbM | ----SASSLYS | 50-56 | 7 | 1059 |
| | Kabat | ----SASSLYS | 50-56 | 7 | 1060 |
| | Contact | LLIYSASSLY- | 46-55 | 10 | 1061 |
| | IMGT | SA | 50-51 | 2 | 1062 |
| CDR-L3 | Chothia | QQWGSSLAT | 89-97 | 9 | 1063 |
| | AbM | QQWGSSLAT | 89-97 | 9 | 1064 |
| | Kabat | QQWGSSLAT | 89-97 | 9 | 1065 |
| | Contact | QQWGSSLA- | 89-96 | 8 | 1066 |
| | IMGT | QQWGSSLAT | 89-97 | 9 | 1067 |

P9-52

| | | | | | |
|---|---|---|---|---|---|
| CDR-H1 | Chothia | GFTFSTY | 26-32 | 7 | 1068 |
| | AbM | GFTFSTYLIH | 26-35 | 10 | 1069 |
| | Kabat | TYLIH | 31-35 | 5 | 1070 |
| | Contact | ----STYLIH | 30-35 | 6 | 1071 |
| | IMGT | GFTFSTYL-- | 26-33 | 8 | 1072 |
| CDR-H2 | Chothia | TPYSGY | 52-57 | 6 | 1073 |
| | AbM | ---AITPYSGYTS | 50-59 | 10 | 1074 |
| | Kabat | ---AITPYSGYTSYADSVKG | 50-66 | 17 | 1075 |
| | Contact | WVAAITPYSGYTS | 47-59 | 13 | 1076 |
| | IMGT | ----ITPYSGYT | 51-58 | 8 | 1077 |
| CDR-H3 | Chothia | --VGYPMVMDY | 99-107 | 9 | 1078 |
| | AbM | --VGYPMVMDY | 99-107 | 9 | 1079 |
| | Kabat | --VGYPMVMDY | 99-107 | 9 | 1080 |
| | Contact | ARVGYPMVMD- | 97-106 | 10 | 1081 |
| | IMGT | ARVGYPMVMDY | 97-107 | 11 | 1082 |
| CDR-L1 | Chothia | RASQSVSSAVA-- | 24-34 | 11 | 1083 |
| | AbM | RASQSVSSAVA-- | 24-34 | 11 | 1084 |
| | Kabat | RASQSVSSAVA-- | 24-34 | 11 | 1085 |
| | Contact | SSAVAWY | 30-36 | 7 | 1086 |
| | IMGT | QSVSSA | 27-32 | 6 | 1087 |
| CDR-L2 | Chothia | SASSLYS | 50-56 | 7 | 1088 |
| | AbM | SASSLYS | 50-56 | 7 | 1089 |
| | Kabat | SASSLYS | 50-56 | 7 | 1090 |
| | Contact | LLIYSASSLY- | 46-55 | 10 | 1091 |
| | IMGT | SA | 50-51 | 2 | 1092 |
| CDR-L3 | Chothia | QQLDYSLAT | 89-97 | 9 | 1093 |
| | AbM | QQLDYSLAT | 89-97 | 9 | 1094 |
| | Kabat | QQLDYSLAT | 89-97 | 9 | 1095 |
| | Contact | QQLDYSLA- | 89-96 | 8 | 1096 |
| | IMGT | QQLDYSLAT | 89-97 | 9 | 1097 |

P9-53

| | | | | | |
|---|---|---|---|---|---|
| CDR-H1 | Chothia | GFTFSRY--- | 26-32 | 7 | 1098 |
| | AbM | GFTFSRYQIH | 26-35 | 10 | 1099 |
| | Kabat | RYQIH | 31-35 | 5 | 1100 |
| | Contact | SRYQIH | 30-35 | 6 | 1101 |
| | IMGT | GFTFSRYQ-- | 26-33 | 8 | 1102 |
| CDR-H2 | Chothia | ASASGT | 52-57 | 6 | 1103 |
| | AbM | ---YIASASGTTS | 50-59 | 10 | 1104 |
| | Kabat | ---YIASASGTTSYADSVKG | 50-66 | 17 | 1105 |
| | Contact | WVAYIASASGTTS | 47-59 | 13 | 1106 |
| | IMGT | ----IASASGTT | 51-58 | 8 | 1107 |
| CDR-H3 | Chothia | --VPYVAMDY | 99-106 | 8 | 1108 |
| | AbM | --VPYVAMDY | 99-106 | 8 | 1109 |
| | Kabat | --VPYVAMDY | 99-106 | 8 | 1110 |
| | Contact | ARVPYVAMD- | 97-105 | 9 | 1111 |
| | IMGT | ARVPYVAMDY | 97-106 | 10 | 1112 |
| CDR-L1 | Chothia | RASQSVSSAVA-- | 24-34 | 11 | 1113 |
| | AbM | RASQSVSSAVA-- | 24-34 | 11 | 1114 |
| | Kabat | RASQSVSSAVA-- | 24-34 | 11 | 1115 |
| | Contact | SSAVAWY | 30-36 | 7 | 1116 |
| | IMGT | QSVSSA | 27-32 | 6 | 1117 |
| CDR-L2 | Chothia | SASSLYS | 50-56 | 7 | 1118 |
| | AbM | SASSLYS | 50-56 | 7 | 1119 |
| | Kabat | SASSLYS | 50-56 | 7 | 1120 |
| | Contact | LLIYSASSLY- | 46-55 | 10 | 1121 |
| | IMGT | SA | 50-51 | 2 | 1122 |
| CDR-L3 | Chothia | QQGYPHPGT | 89-97 | 9 | 1123 |
| | AbM | QQGYPHPGT | 89-97 | 9 | 1124 |
| | Kabat | QQGYPHPGT | 89-97 | 9 | 1125 |
| | Contact | QQGYPHPG- | 89-96 | 8 | 1126 |
| | IMGT | QQGYPHPGT | 89-97 | 9 | 1127 |

P9-56

| | | | | | |
|---|---|---|---|---|---|
| CDR-H1 | Chothia | GFTFSSY--- | 26-32 | 7 | 1128 |
| | AbM | GFTFSSYYIH | 26-35 | 10 | 1129 |
| | Kabat | SYYIH | 31-35 | 5 | 1130 |
| | Contact | SSYYIH | 30-35 | 6 | 1131 |
| | IMGT | GFTFSSYY-- | 26-33 | 8 | 1132 |
| CDR-H2 | Chothia | DSSGKY | 52-57 | 6 | 1133 |
| | AbM | ---YIDSSGKYTD | 50-59 | 10 | 1134 |
| | Kabat | ---YIDSSGKYTDYADSVKG | 50-66 | 17 | 1135 |
| | Contact | WVAYIDSSGKYTD | 47-59 | 13 | 1136 |
| | IMGT | ----IDSSGKYT | 51-58 | 8 | 1137 |
| CDR-H3 | Chothia | --YAYPGVMDY | 99-107 | 9 | 1138 |
| | AbM | --YAYPGVMDY | 99-107 | 9 | 1139 |
| | Kabat | --YAYPGVMDY | 99-107 | 9 | 1140 |
| | Contact | ARYAYPGVMD- | 97-106 | 10 | 1141 |
| | IMGT | ARYAYPGVMDY | 97-107 | 11 | 1142 |
| CDR-L1 | Chothia | RASQSVSSAVA-- | 24-34 | 11 | 1143 |
| | AbM | RASQSVSSAVA-- | 24-34 | 11 | 1144 |
| | Kabat | RASQSVSSAVA-- | 24-34 | 11 | 1145 |
| | Contact | SSAVAWY | 30-36 | 7 | 1146 |
| | IMGT | QSVSSA | 27-32 | 6 | 1147 |
| CDR-L2 | Chothia | ----SASSLYS | 50-56 | 7 | 1148 |
| | AbM | ----SASSLYS | 50-56 | 7 | 1149 |
| | Kabat | ----SASSLYS | 50-56 | 7 | 1150 |
| | Contact | LLIYSASSLY- | 46-55 | 10 | 1151 |
| | IMGT | SA | 50-51 | 2 | 1152 |
| CDR-L3 | Chothia | QQYDYSLWT | 89-97 | 9 | 1153 |
| | AbM | QQYDYSLWT | 89-97 | 9 | 1154 |
| | Kabat | QQYDYSLWT | 89-97 | 9 | 1155 |
| | Contact | QQYDYSLW- | 89-96 | 8 | 1156 |
| | IMGT | QQYDYSLWT | 89-97 | 9 | 1157 |

P9-57

| | | | | | |
|---|---|---|---|---|---|
| CDR-H1 | Chothia | GFTFSSY | 26-32 | 7 | 1158 |
| | AbM | GFTFSSYYIH | 26-35 | 10 | 1159 |
| | Kabat | SYYIH | 31-35 | 5 | 1160 |
| | Contact | ----SSYYIH | 30-35 | 6 | 1161 |
| | IMGT | GFTFSSYY-- | 26-33 | 8 | 1162 |
| CDR-H2 | Chothia | YPSGGY | 52-57 | 6 | 1163 |
| | AbM | ---TIYPSGGYTY | 50-59 | 10 | 1164 |
| | Kabat | ---TIYPSGGYTYYADSVKG | 50-66 | 17 | 1165 |
| | Contact | WVATIYPSGGYTY | 47-59 | 13 | 1166 |
| | IMGT | IYPSGGYT | 51-58 | 8 | 1167 |
| CDR-H3 | Chothia | --YSYPGVLDY | 99-107 | 9 | 1168 |
| | AbM | --YSYPGVLDY | 99-107 | 9 | 1169 |
| | Kabat | --YSYPGVLDY | 99-107 | 9 | 1170 |
| | Contact | ARYSYPGVLD- | 97-106 | 10 | 1171 |
| | IMGT | ARYSYPGVLDY | 97-107 | 11 | 1172 |
| CDR-L1 | Chothia | RASQSVSSAVA-- | 24-34 | 11 | 1173 |
| | AbM | RASQSVSSAVA-- | 24-34 | 11 | 1174 |
| | Kabat | RASQSVSSAVA-- | 24-34 | 11 | 1175 |
| | Contact | SSAVAWY | 30-36 | 7 | 1176 |
| | IMGT | QSVSSA | 27-32 | 6 | 1177 |
| CDR-L2 | Chothia | SASSLYS | 50-56 | 7 | 1178 |
| | AbM | SASSLYS | 50-56 | 7 | 1179 |
| | Kabat | ----SASSLYS | 50-56 | 7 | 1180 |
| | Contact | LLIYSASSLY- | 46-55 | 10 | 1181 |
| | IMGT | ----SA | 50-51 | 2 | 1182 |
| CDR-L3 | Chothia | QQSSSFLWT | 89-97 | 9 | 1183 |
| | AbM | QQSSSFLWT | 89-97 | 9 | 1184 |
| | Kabat | QQSSSFLWT | 89-97 | 9 | 1185 |
| | Contact | QQSSSFLW- | 89-96 | 8 | 1186 |
| | IMGT | QQSSSFLWT | 89-97 | 9 | 1187 |

Table 6 presents full immunoglobulin heavy and full immunoglobulin light chain sequences, and the VH and VL sequences, of various ABS candidates formatted into a bivalent monospecific human full-length IgG1 architecture.

TABLE 6
Full chain sequences and VH/VL sequences of candidate GAL9 ABS clones and IgG
formatted antibodies comprising GAL9 ABSs

| ABS | | | | |
|---|---|---|---|---|
| clone | Full IgG Light Chain | Full IgG Heavy Chain | VH sequence | VL sequence |
| P9-01 | EVQLVESGGGLVQPGGS | DIQMTQSPSSLSASVGDRV | EVQLVESGGGL | DIQMTQSPSS |
| | LRLSCAASGFTFSSYWIH | TITCRASQSVSSAVAWYQQ | VQPGGSLRLSC | LSASVGDRV |
| | WVRQAPGKGLEWVAWI | KPGKAPKLLIYSASSLYSG | AASGFTFSSYWI | TITCRASQSV |
| | DPDYGTTSYADSVKGRF | VPSRFSGSRSGTDFTLTISS | HWVRQAPGKG | SSAVAWYQQ |
| | TISADTSKNTAYLQMNS | LQPEDFATYYCQQQVSDL | LEWVAWIDPD | KPGKAPKLLI |
| | LRAEDTAVYYCARAGIS | LTFGQGTKVEIKRTVAAPS | YGTTSYADSVK | YSASSLYSGV |
| | YVFDYWGQGTLVTVSS | VFIFPPSDSQLKSGTASVVC | GRFTISADTSKN | PSRFSGSRSG |
| | ASTKGPSVFPLAPSSKST | LLNNFYPREAKVQWKVDN | TAYLQMNSLRA | TDFTLTISSLQ |
| | SGGTAALGCLVKDYFPE | ALQSGNSQESVTEQDSKDS | EDTAVYYCAR | PEDFATYYC |
| | PVTVSWNSGALTSGVHT | TYSLSSTLTLSKADYEKHK | AGISYVFDYWG | QQQVSDLLT |
| | FPAVLQSSGLYSLSSVVT | VYACEVTHQGLSSPVTKSF | QGTLVTVSS | FGQGTKVEIK |
| | VPSSSLGTQTYICNVNH | NRGEC | | RTV |
| | KPSNTKVDKKVEPKSCD | | | |
| | KTHTCPPCPAPELLGGPS | | | |
| | VFLFPPKPKDTLMISRTP | | | |
| | EVTCVVVDVSHEDPEVK | | | |
| | FNWYVDGVEVHNAKTK | | | |
| | PREEQYNSTYRVVSVLT | | | |
| | VLHQDWLNGKEYKCKV | | | |
| | SNKALPAPIEKTISKAKG | | | |
| | QPREPQVYTLPPSRDELT | | | |
| | KNQVSLTCLVKGFYPSD | | | |
| | IAVEWESNGQPENNYKT | | | |
| | TPPVLDSDGSFFLYSKLT | | | |
| | VDKSRWQQGNVFSCSV | | | |
| | MHEALHNHYTQKSLSLS | | | |
| | PGK | | | |
| P9-02A | EVQLVESGGGLVQPGGS | DIQMTQSPSSLSASVGDRV | EVQLVESGGGL | DIQMTQSPSS |
| | LRLSCAASGFTFSSYWIH | TITCRASQSVSSAVAWYQQ | VQPGGSLRLSC | LSASVGDRV |
| | WVRQAPGKGLEWVAWI | KPGKAPKLLIYSASSLYSG | AASGFTFSSYWI | TITCRASQSV |
| | DPDYGTTSYADSVKGRF | VPSRFSGSRSGTDFTLTISS | HWVRQAPGKG | SSAVAWYQQ |
| | TISADTSKNTAYLQMNS | LQPEDFATYYCQQSYPTLG | LEWVAWIDPD | KPGKAPKLLI |
| | LRAEDTAVYYCARAQY | TFGQGTKVEIKRTVAAPSV | YGTTSYADSVK | YSASSLYSGV |
| | VPGLDYWGQGTLVTVS | FIFPPSDSQLKSGTASVVCL | GRFTISADTSKN | PSRFSGSRSG |
| | SASTKGPSVFPLAPSSKS | LNNFYPREAKVQWKVDN | TAYLQMNSLRA | TDFTLTISSLQ |
| | TSGGTAALGCLVKDYFP | ALQSGNSQESVTEQDSKDS | EDTAVYYCAR | PEDFATYYC |
| | EPVTVSWNSGALTSGVH | TYSLSSTLTLSKADYEKHK | AQYVPGLDYW | QQSYPTLGTF |
| | TFPAVLQSSGLYSLSSVV | VYACEVTHQGLSSPVTKSF | GQGTLVTVSS | GQGTKVEIKR |
| | TVPSSSLGTQTYICNVNH | NRGEC | | TV |
| | KPSNTKVDKKVEPKSCD | | | |
| | KTHTCPPCPAPELLGGPS | | | |
| | VFLFPPKPKDTLMISRTP | | | |
| | EVTCVVVDVSHEDPEVK | | | |
| | FNWYVDGVEVHNAKTK | | | |
| | PREEQYNSTYRVVSVLT | | | |
| | VLHQDWLNGKEYKCKV | | | |
| | SNKALPAPIEKTISKAKG | | | |
| | QPREPQVYTLPPSRDELT | | | |
| | KNQVSLTCLVKGFYPSD | | | |
| | IAVEWESNGQPENNYKT | | | |
| | TPPVLDSDGSFFLYSKLT | | | |
| | VDKSRWQQGNVFSCSV | | | |
| | MHEALHNHYTQKSLSLS | | | |
| | PGK | | | |
| P9-03 | EVQLVESGGGLVQPGGS | DIQMTQSPSSLSASVGDRV | EVQLVESGGGL | DIQMTQSPSS |
| | LRLSCAASGFTFSGYYIH | TITCRASQSVSSAVAWYQQ | VQPGGSLRLSC | LSASVGDRV |
| | WVRQAPGKGLEWVAVI | KPGKAPKLLIYSASSLYSG | AASGFTFSGYYI | TITCRASQSV |
| | SPYSGYTSYADSVKGRF | VPSRFSGSRSGTDFTLTISS | HWVRQAPGKG | SSAVAWYQQ |
| | TISADTSKNTAYLQMNS | LQPEDFATYYCQQGGSFPY | LEWVAVISPYS | KPGKAPKLLI |
| | LRAEDTAVYYCARATY | TFGQGTKVEIKRTVAAPSV | GYTSYADSVKG | YSASSLYSGV |
| | MVPYGFDYWGQGTLVT | FIFPPSDSQLKSGTASVVCL | RFTISADTSKNT | PSRFSGSRSG |
| | VSSASTKGPSVFPLAPSS | LNNFYPREAKVQWKVDN | AYLQMNSLRAE | TDFTLTISSLQ |
| | KSTSGGTAALGCLVKDY | ALQSGNSQESVTEQDSKDS | DTAVYYCARA | PEDFATYYC |
| | FPEPVTVSWNSGALTSG | TYSLSSTLTLSKADYEKHK | TYMVPYGFDY | QQGGSFPYTF |
| | VHTFPAVLQSSGLYSLSS | VYACEVTHQGLSSPVTKSF | WGQGTLVTVSS | GQGTKVEIKR |
| | VVTVPSSSLGTQTYICNV | NRGEC | | TV |
| | NHKPSNTKVDKKVEPKS | | | |
| | CDKTHTCPPCPAPELLG | | | |
| | GPSVFLFPPKPKDTLMIS | | | |
| | RTPEVTCVVVDVSHEDP | | | |
| | EVKFNWYVDGVEVHNA | | | |
| | KTKPREEQYNSTYRVVS | | | |
| | VLTVLHQDWLNGKEYK | | | |
| | CKVSNKALPAPIEKTISK | | | |
| | AKGQPREPQVYTLPPSR | | | |
| | DELTKNQVSLTCLVKGF | | | |
| | YPSDIAVEWESNGQPEN | | | |
| | NYKTTPPVLDSDGSFFL | | | |
| | YSKLTVDKSRWQQGNV | | | |
| | FSCSVMHEALHNHYTQ | | | |
| | KSLSLSPGK | | | |
| P9-06 | EVQLVESGGGLVQPGGS | DIQMTQSPSSLSASVGDRV | ′EVQLVESGGGL | DIQMTQSPSS |
| | LRLSCAASGFTFAYYGIH | TITCRASQSVSSAVAWYQQ | VQPGGSLRLSC | LSASVGDRV |
| | WVRQAPGKGLEWVAYI | KPGKAPKLLIYSASSLYSG | AASGFTFAYYG | TITCRASQSV |
| | YPHGYITDYADSVKGRF | VPSRFSGSRSGTDFTLTISS | IHWVRQAPGKG | SSAVAWYQQ |
| | TISADTSKNTAYLQMNS | LQPEDFATYYCQQHFSSPG | LEWVAYIYPHG | KPGKAPKLLI |
| | LRAEDTAVYYCARDSG | TFGQGTKVEIKRTVAAPSV | YITDYADSVKG | YSASSLYSGV |
| | VPYYWAVLDYWGQGTL | FIFPPSDSQLKSGTASVVCL | RFTISADTSKNT | PSRFSGSRSG |
| | VTVSSASTKGPSVFPLAP | LNNFYPREAKVQWKVDN | AYLQMNSLRAE | TDFTLTISSLQ |
| | SSKSTSGGTAALGCLVK | ALQSGNSQESVTEQDSKDS | DTAVYYCARDS | PEDFATYYC |
| | DYFPEPVTVSWNSGALT | TYSLSSTLTLSKADYEKHK | GVPYYWAVLD | QQHFSSPGTF |
| | SGVHTFPAVLQSSGLYS | VYACEVTHQGLSSPVTKSF | YWGQGTLVTV | GQGTKVEIKR |
| | LSSVVTVPSSSLGTQTYI | NRGEC | SS | TV |
| | CNVNHKPSNTKVDKKV | | | |
| | EPKSCDKTHTCPPCPAPE | | | |
| | LLGGPSVFLFPPKPKDTL | | | |
| | MISRTPEVTCVVVDVSH | | | |
| | EDPEVKFNWYVDGVEV | | | |
| | HNAKTKPREEQYNSTYR | | | |
| | VVSVLTVLHQDWLNGK | | | |
| | EYKCKVSNKALPAPIEK | | | |
| | TISKAKGQPREPQVYTLP | | | |
| | PSRDELTKNQVSLTCLV | | | |
| | KGFYPSDIAVEWESNGQ | | | |
| | PENNYKTTPPVLDSDGS | | | |
| | FFLYSKLTVDKSRWQQG | | | |
| | NVFSCSVMHEALHNHY | | | |
| | TQKSLSLSPGK | | | |
| P9-07 | EVQLVESGGGLVQPGGS | DIQMTQSPSSLSASVGDRV | EVQLVESGGGL | DIQMTQSPSS |
| | LRLSCAASGFTFSSYYIH | TITCRASQSVSSAVAWYQQ | VQPGGSLRLSC | LSASVGDRV |
| | WVRQAPGKGLEWVAYI | KPGKAPKLLIYSASSLYSG | AASGFTFSSYYI | TITCRASQSV |
| | SPYGGDTSYADSVKGRF | VPSRFSGSRSGTDFTLTISS | HWVRQAPGKG | SSAVAWYQQ |
| | TISADTSKNTAYLQMNS | LQPEDFATYYCQQWTSTL | LEWVAYISPYG | KPGKAPKLLI |
| | LRAEDTAVYYCARDSY | WTFGQGTKVEIKRTVAAPS | GDTSYADSVKG | YSASSLYSGV |
| | MSYIDGFDYWGQGTLV | VFIFPPSDSQLKSGTASVVC | RFTISADTSKNT | PSRFSGSRSG |
| | TVSSASTKGPSVFPLAPS | LLNNFYPREAKVQWKVDN | AYLQMNSLRAE | TDFTLTISSLQ |
| | SKSTSGGTTALGCLVKD | ALQSGNSQESVTEQDSKDS | DTAVYYCARDS | PEDFATYYC |
| | YFPEPVTVSWNSGALTS | TYSLSSTLTLSKADYEKHK | YMSYIDGFDY | QQWTSTLWT |
| | GVHTFPAVLQSSGLYSL | VYACEVTHQGLSSPVTKSF | WGQGTLVTVSS | FGQGTKVEIK |
| | SSVVTVPSSSLGTQTYIC | NRGEC | | RTV |
| | NVNHKPSNTKVDKKVE | | | |
| | PKSCDKTHTCPPCPAPEL | | | |
| | LGGPSVFLFPPKPKDTL | | | |
| | MISRTPEVTCVVVDVSH | | | |
| | EDPEVKFNWYVDGVEV | | | |
| | HNAKTKPREEQYNSTYR | | | |
| | VVSVLTVLHQDWLNGK | | | |
| | EYKCKVSNKALPAPIEK | | | |
| | TISKAKGQPREPQVYTLP | | | |
| | PSRDELTKNQVSLTCLV | | | |
| | KGFYPSDIAVEWESNGQ | | | |
| | PENNYKTTPPVLDSDGS | | | |
| | FFLYSKLTVDKSRWQQG | | | |
| | NVFSCSVMHEALHNHY | | | |
| | TQKSLSLSPGK | | | |
| P9-11 | EVQLVESGGGLVQPGGS | DIQMTQSPSSLSASVGDRV | EVQLVESGGGL | DIQMTQSPSS |
| | LRLSCAASGFTFSSYYIH | TITCRASQSVSSAVAWYQQ | VQPGGSLRLSC | LSASVGDRV |
| | WVRQAPGKGLEWVAYI | KPGKAPKLLIYSASSLYSG | AASGFTFSSYYI | TITCRASQSV |
| | SPSGGYTYYADSVKGRF | VPSRFSGSRSGTDFTLTISS | HWVRQAPGKG | SSAVAWYQQ |
| | TISADTSKNTAYLQMNS | LQPEDFATYYCQQYYPSPS | LEWVAYISPSG | KPGKAPKLLI |
| | LRAEDTAVYYCARGAV | TFGQGTKVEIKRTVAAPSV | GYTYYADSVK | YSASSLYSGV |
| | LYSSAMDYWGQGTLVT | FIFPPSDSQLKSGTASVVCL | GRFTISADTSKN | PSRFSGSRSG |
| | VSSASTKGPSVFPLAPSS | LNNFYPREAKVQWKVDN | TAYLQMNSLRA | TDFTLTISSLQ |
| | KSTSGGTAALGCLVKDY | ALQSGNSQESVTEQDSKDS | EDTAVYYCAR | PEDFATYYC |
| | FPEPVTVSWNSGALTSG | TYSLSSTLTLSKADYEKHK | GAVLYSSAMD | QQYYPSPSTF |
| | VHTFPAVLQSSGLYSLSS | VYACEVTHQGLSSPVTKSF | YWGQGTLVTV | GQGTKVEIKR |
| | VVTVPSSSLGTQTYICNV | NRGEC | SS | TV |
| | NHKPSNTKVDKKVEPKS | | | |
| | CDKTHTCPPCPAPELLG | | | |
| | GPSVFLFPPKPKDTLMIS | | | |
| | RTPEVTCVVVDVSHEDP | | | |
| | EVKFNWYVDGVEVHNA | | | |
| | KTKPREEQYNSTYRVVS | | | |
| | VLTVLHQDWLNGKEYK | | | |
| | CKVSNKALPAPIEKTISK | | | |
| | AKGQPREPQVYTLPPSR | | | |
| | DELTKNQVSLTCLVKGF | | | |
| | YPSDIAVEWESNGQPEN | | | |
| | NYKTTPPVLDSDGSFFL | | | |
| | YSKLTVDKSRWQQGNV | | | |
| | FSCSVMHEALHNHYTQ | | | |
| | KSLSLSPGK | | | |
| P9-12 | EVQLVESGGGLVQPGGS | DIQMTQSPSSLSASVGDRV | EVQLVESGGGL | DIQMTQSPSS |
| | LRLSCAASGFTFSSYWIH | TITCRASQSVSSAVAWYQQ | VQPGGSLRLSC | LSASVGDRV |
| | WVRQAPGKGLEWVASI | KPGKAPKLLIYSASSLYSG | AASGFTFSSYWI | TITCRASQSV |
| | ASYFGQTYYADSVKGRF | VPSRFSGSRSGTDFTLTISS | HWVRQAPGKG | SSAVAWYQQ |
| | TISADTSKNTAYLQMNS | LQPEDFATYYCQQEYGRP | LEWVASIASYF | KPGKAPKLLI |
| | LRAEDTAVYYCARGFG | YTFGQGTKVEIKRTVAAPS | GQTYYADSVK | YSASSLYSGV |
| | YAAMDYWGQGTLVTVS | VFIFPPSDSQLKSGTASVVC | GRFTISADTSKN | PSRFSGSRSG |
| | SASTKGPSVFPLAPSSKS | LLNNFYPREAKVQWKVDN | TAYLQMNSLRA | TDFTLTISSLQ |
| | TSGGTAALGCLVKDYFP | ALQSGNSQESVTEQDSKDS | EDTAVYYCAR | PEDFATYYC |
| | EPVTVSWNSGALTSGVH | TYSLSSTLTLSKADYEKHK | GFGYAAMDYW | QQEYGRPYT |
| | TFPAVLQSSGLYSLSSVV | VYACEVTHQGLSSPVTKSF | GQGTLVTVSS | FGQGTKVEIK |
| | TVPSSSLGTQTYICNVNH | NRGEC | | RTV |
| | KPSNTKVDKKVEPKSCD | | | |
| | KTHTCPPCPAPELLGGPS | | | |
| | VFLFPPKPKDTLMISRTP | | | |
| | EVTCVVVDVSHEDPEVK | | | |
| | FNWYVDGVEVHNAKTK | | | |
| | PREEQYNSTYRVVSVLT | | | |
| | VLHQDWLNGKEYKCKV | | | |
| | SNKALPAPIEKTISKAKG | | | |
| | QPREPQVYTLPPSRDELT | | | |
| | KNQVSLTCLVKGFYPSD | | | |
| | IAVEWESNGQPENNYKT | | | |
| | TPPVLDSDGSFFLYSKLT | | | |
| | VDKSRWQQGNVFSCSV | | | |
| | MHEALHNHYTQKSLSLS | | | |
| | PGK | | | |
| P9-14 | EVQLVESGGGLVQPGGS | DIQMTQSPSSLSASVGDRV | EVQLVESGGGL | DIQMTQSPSS |
| | LRLSCAASGFTFGSYYIH | TITCRASQSVSSAVAWYQQ | VQPGGSLRLSC | LSASVGDRV |
| | WVRQAPGKGLEWVADI | KPGKAPKLLIYSASSLYSG | AASGFTFGSYYI | TITCRASQSV |
| | YPYFSSTYYADSVKGRF | VPSRFSGSRSGTDFTLTISS | HWVRQAPGKG | SSAVAWYQQ |
| | TISADTSKNTAYLQMNS | LQPEDFATYYCQQHASGPL | LEWVADIYPYF | KPGKAPKLLI |
| | LRAEDTAVYYCARGSHF | TFGQGTKVEIKRTVAAPSV | SSTYYADSVKG | YSASSLYSGV |
| | GFDYWGQGTLVTVSSAS | FIFPPSDSQLKSGTASVVCL | RFTISADTSKNT | PSRFSGSRSG |
| | TKGPSVFPLAPSSKSTSG | LNNFYPREAKVQWKVDN | AYLQMNSLRAE | TDFTLTISSLQ |
| | GTAALGCLVKDYFPEPV | ALQSGNSQESVTEQDSKDS | DTAVYYCARGS | PEDFATYYC |
| | TVSWNSGALTSGVHTFP | TYSLSSTLTLSKADYEKHK | HFGFDYWGQG | QQHASGPLTF |
| | AVLQSSGLYSLSSVVTV | VYACEVTHQGLSSPVTKSF | TLVTVSS | GQGTKVEIKR |
| | PSSSLGTQTYICNVNHKP | NRGEC | | TV |
| | SNTKVDKKVEPKSCDKT | | | |
| | HTCPPCPAPELLGGPSVF | | | |
| | LFPPKPKDTLMISRTPEV | | | |
| | TCVVVDVSHEDPEVKFN | | | |
| | WYVDGVEVHNAKTKPR | | | |
| | EEQYNSTYRVVSVLTVL | | | |
| | HQDWLNGKEYKCKVSN | | | |
| | KALPAPIEKTISKAKGQP | | | |
| | REPQVYTLPPSRDELTK | | | |
| | NQVSLTCLVKGFYPSDI | | | |
| | AVEWESNGQPENNYKT | | | |
| | TPPVLDSDGSFFLYSKLT | | | |
| | VDKSRWQQGNVFSCSV | | | |
| | MHEALHNHYTQKSLSL | | | |
| P9-23 | EVQLVESGGGLVQPGGS | DIQMTQSPSSLSASVGDRV | EVQLVESGGGL | DIQMTQSPSS |
| | LRLSCAASGFTFSQYYIH | TITCRASQSVSSAVAWYQQ | VQPGGSLRLSC | LSASVGDRV |
| | WVRQAPGKGLEWVATI | KPGKAPKLLIYSASSLYSG | AASGFTFSQYYI | TITCRASQSV |
| | YPRGGYTFYADSVKGRF | VPSRFSGSRSGTDFTLTISS | HWVRQAPGKG | SSAVAWYQQ |
| | TISADTSKNTAYLQMNS | LQPEDFATYYCQQWSVYL | LEWVATIYPRG | KPGKAPKLLI |
| | LRAEDTAVYYCARKSY | ETFGQGTKVEIKRTVAAPS | GYTFYADSVKG | YSASSLYSGV |
| | WGMDYWGQGTLVTVSS | VFIFPPSDSQLKSGTASVVC | RFTISADTSKNT | PSRFSGSRSG |
| | ASTKGPSVFPLAPSSKST | LLNNFYPREAKVQWKVDN | AYLQMNSLRAE | TDFTLTISSLQ |
| | SGGTAALGCLVKDYFPE | ALQSGNSQESVTEQDSKDS | DTAVYYCARKS | PEDFATYYC |
| | PVTVSWNSGALTSGVHT | TYSLSSTLTLSKADYEKHK | YWGMDYWGQ | QQWSVYLET |
| | FPAVLQSSGLYSLSSVVT | VYACEVTHQGLSSPVTKSF | GTLVTVSS | FGQGTKVEIK |
| | VPSSSLGTQTYICNVNH | NRGEC | | RTV |
| | KPSNTKVDKKVEPKSCD | | | |
| | KTHTCPPCPAPELLGGPS | | | |
| | VFLFPPKPKDTLMISRTP | | | |
| | EVTCVVVDVSHEDPEVK | | | |
| | FNWYVDGVEVHNAKTK | | | |
| | PREEQYNSTYRVVSVLT | | | |
| | VLHQDWLNGKEYKCKV | | | |
| | SNKALPAPIEKTISKAKG | | | |
| | QPREPQVYTLPPSRDELT | | | |
| | KNQVSLTCLVKGFYPSD | | | |
| | IAVEWESNGQPENNYKT | | | |
| | TPPVLDSDGSFFLYSKLT | | | |
| | VDKSRWQQGNVFSCSV | | | |
| | MHEALHNHYTQKSLSLS | | | |
| | PGK | | | |
| P9-24 | EVQLVESGGGLVQPGGS | DIQMTQSPSSLSASVGDRV | EVQLVESGGGL | DIQMTQSPSS |
| | LRLSCAASGFTFSSYFIH | TITCRASQSVSSAVAWYQQ | VQPGGSLRLSC | LSASVGDRV |
| | WVRQAPGKGLEWVASI | KPGKAPKLLIYSASSLYSG | AASGFTFSSYFI | TITCRASQSV |
| | YPTSHSTSYADSVKGRF | VPSRFSGSRSGTDFTLTISS | HWVRQAPGKG | SSAVAWYQQ |
| | TISADTSKNTAYLQMNS | LQPEDFATYYCQQVDSRL | LEWVASIYPTS | KPGKAPKLLI |
| | LRAEDTAVYYCARLGYP | ATFGQGTKVEIKRTVAAPS | HSTSYADSVKG | YSASSLYSGV |
| | GVMDYWGQGTLVTVSS | VFIFPPSDSQLKSGTASVVC | RFTISADTSKNT | PSRFSGSRSG |
| | ASTKGPSVFPLAPSSKST | LLNNFYPREAKVQWKVDN | AYLQMNSLRAE | TDFTLTISSLQ |
| | SGGTAALGCLVKDYFPE | ALQSGNSQESVTEQDSKDS | DTAVYYCARL | PEDFATYYC |
| | PVTVSWNSGALTSGVHT | TYSLSSTLTLSKADYEKHK | GYPGVMDYWG | QQVDSRLAT |
| | FPAVLQSSGLYSLSSVVT | VYACEVTHQGLSSPVTKSF | QGTLVTVSS | FGQGTKVEIK |
| | VPSSSLGTQTYICNVNH | NRGEC | | RTV |
| | KPSNTKVDKKVEPKSCD | | | |
| | KTHTCPPCPAPELLGGPS | | | |
| | VFLFPPKPKDTLMISRTP | | | |
| | EVTCVVVDVSHEDPEVK | | | |
| | FNWYVDGVEVHNAKTK | | | |
| | PREEQYNSTYRVVSVLT | | | |
| | VLHQDWLNGKEYKCKV | | | |
| | SNKALPAPIEKTISKAKG | | | |
| | QPREPQVYTLPPSRDELT | | | |
| | KNQVSLTCLVKGFYPSD | | | |
| | IAVEWESNGQPENNYKT | | | |
| | TPPVLDSDGSFFLYSKLT | | | |
| | VDKSRWQQGNVFSCSV | | | |
| | MHEALHNHYTQKSLSLS | | | |
| | PGK | | | |
| P9-25 | EVQLVESGGGLVQPGGS | DIQMTQSPSSLSASVGDRV | EVQLVESGGGL | DIQMTQSPSS |
| | LRLSCAASGFTFSSYYIH | TITCRASQSVSSAVAWYQQ | VQPGGSLRLSC | LSASVGDRV |
| | WVRQAPGKGLEWVASI | KPGKAPKLLIYSASSLYSG | AASGFTFSSYYI | TITCRASQSV |
| | YPYGSYTYYADSVKGRF | VPSRFSGSRSGTDFTLTISS | HWVRQAPGKG | SSAVAWYQQ |
| | TISADTSKNTAYLQMNS | LQPEDFATYYCQQWAPDL | LEWVASIYPYG | KPGKAPKLLI |
| | LRAEDTAVYYCARLGYS | TTFGQGTKVEIKRTVAAPS | SYTYYADSVKG | YSASSLYSGV |
| | SGMDYWGQGTLVTVSS | VFIFPPSDSQLKSGTASVVC | RFTISADTSKNT | PSRFSGSRSG |
| | ASTKGPSVFPLAPSSKST | LLNNFYPREAKVQWKVDN | AYLQMNSLRAE | TDFTLTISSLQ |
| | SGGTAALGCLVKDYFPE | ALQSGNSQESVTEQDSKDS | DTAVYYCARL | PEDFATYYC |
| | PVTVSWNSGALTSGVHT | TYSLSSTLTLSKADYEKHK | GYSSGMDYWG | QQWAPDLTT |
| | FPAVLQSSGLYSLSSVVT | VYACEVTHQGLSSPVTKSF | QGTLVTVSS | FGQGTKVEIK |
| | VPSSSLGTQTYICNVNH | NRGEC | | RTV |
| | KPSNTKVDKKVEPKSCD | | | |
| | KTHTCPPCPAPELLGGPS | | | |
| | VFLFPPKPKDTLMISRTP | | | |
| | EVTCVVVDVSHEDPEVK | | | |
| | FNWYVDGVEVHNAKTK | | | |
| | PREEQYNSTYRVVSVLT | | | |
| | VLHQDWLNGKEYKCKV | | | |
| | SNKALPAPIEKTISKAKG | | | |
| | QPREPQVYTLPPSRDELT | | | |
| | KNQVSLTCLVKGFYPSD | | | |
| | IAVEWESNGQPENNYKT | | | |
| | TPPVLDSDGSFFLYSKLT | | | |
| | VDKSRWQQGNVFSCSV | | | |
| | MHEALHNHYTQKSLSLS | | | |
| | PGK | | | |
| P9-26 | EVQLVESGGGLVQPGGS | DIQMTQSPSSLSASVGDRV | EVQLVESGGGL | DIQMTQSPSS |
| NEG. | LRLSCAASGFTFSSYYIH | TITCRASQSVSSAVAWYQQ | VQPGGSLRLSC | LSASVGDRV |
| CON | WVRQAPGKGLEWVAWI | KPGKAPKLLIYSASSLYSG | AASGFTFSSYYI | TITCRASQSV |
| | ESSSSHTDYADSVKGRF | VPSRFSGSRSGTDFTLTISS | HWVRQAPGKG | SSAVAWYQQ |
| | TISADTSKNTAYLQMNS | LQPEDFATYYCQQYSSSLY | LEWVAWIESSS | KPGKAPKLLI |
| | LRAEDTAVYYCARLPYK | TFGQGTKVEIKRTVAAPSV | SHTDYADSVKG | YSASSLYSGV |
| | YYYLGVFDYWGQGTLV | FIFPPSDSQLKSGTASVVCL | RFTISADTSKNT | PSRFSGSRSG |
| | TVSSASTKGPSVFPLAPS | LNNFYPREAKVQWKVDN | AYLQMNSLRAE | TDFTLTISSLQ |
| | SKSTSGGTAALGCLVKD | ALQSGNSQESVTEQDSKDS | DTAVYYCARLP | PEDFATYYC |
| | YFPEPVTVSWNSGALTS | TYSLSSTLTLSKADYEKHK | YKYYYLGVFD | QQYSSSLYTF |
| | GVHTFPAVLQSSGLYSL | VYACEVTHQGLSSPVTKSF | YWGQGTLVTV | GQGTKVEIKR |
| | SSVVTVPSSSLGTQTYIC | NRGEC | SS | TVA |
| | NVNHKPSNTKVDKKVE | | | |
| | PKSCDKTHTCPPCPAPEL | | | |
| | LGGPSVFLFPPKPKDTL | | | |
| | MISRTPEVTCVVVDVSH | | | |
| | EDPEVKFNWYVDGVEV | | | |
| | HNAKTKPREEQYNSTYR | | | |
| | VVSVLTVLHQDWLNGK | | | |
| | EYKCKVSNKALPAPIEK | | | |
| | TISKAKGQPREPQVYTLP | | | |
| | PSRDELTKNQVSLTCLV | | | |
| | KGFYPSDIAVEWESNGQ | | | |
| | PENNYKTTPPVLDSDGS | | | |
| | FFLYSKLTVDKSRWQQG | | | |
| | NVFSCSVMHEALHNHY | | | |
| | TQKSLSLSPGK | | | |
| P9-29 | EVQLVESGGGLVQPGGS | DIQMTQSPSSLSASVGDRV | EVQLVESGGGL | DIQMTQSPSS |
| | LRLSCAASGFTFSSYAIH | TITCRASQSVSSAVAWYQQ | VQPGGSLRLSC | LSASVGDRV |
| | WVRQAPGKGLEWVAYI | KPGKAPKLLIYSASSLYSG | AASGFTFSSYAI | TITCRASQSV |
| | APGGSYTYYADSVKGRF | VPSRFSGSRSGTDFTLTISS | HWVRQAPGKG | SSAVAWYQQ |
| | TISADTSKNTAYLQMNS | LQPEDFATYYCQQGYSSLL | LEWVAYIAPGG | KPGKAPKLLI |
| | LRAEDTAVYYCARLSYP | TFGQGTKVEIKRTVAAPSV | SYTYYADSVKG | YSASSLYSGV |
| | GVMDYWGQGTLVTVSS | FIFPPSDSQLKSGTASVVCL | RFTISADTSKNT | PSRFSGSRSG |
| | ASTKGPSVFPLAPSSKST | LNNFYPREAKVQWKVDN | AYLQMNSLRAE | TDFTLTISSLQ |
| | SGGTAALGCLVKDYFPE | ALQSGNSQESVTEQDSKDS | DTAVYYCARLS | PEDFATYYC |
| | PVTVSWNSGALTSGVHT | TYSLSSTLTLSKADYEKHK | YPGVMDYWGQ | QQGYSSLLTF |
| | FPAVLQSSGLYSLSSVVT | VYACEVTHQGLSSPVTKSF | GTLVTVSS | GQGTKVEIKR |
| | VPSSSLGTQTYICNVNH | NRGEC | | TV |
| | KPSNTKVDKKVEPKSCD | | | |
| | KTHTCPPCPAPELLGGPS | | | |
| | VFLFPPKPKDTLMISRTP | | | |
| | EVTCVVVDVSHEDPEVK | | | |
| | FNWYVDGVEVHNAKTK | | | |
| | PREEQYNSTYRVVSVLT | | | |
| | VLHQDWLNGKEYKCKV | | | |
| | SNKALPAPIEKTISKAKG | | | |
| | QPREPQVYTLPPSRDELT | | | |
| | KNQVSLTCLVKGFYPSD | | | |
| | IAVEWESNGQPENNYKT | | | |
| | TPPVLDSDGSFFLYSKLT | | | |
| | VDKSRWQQGNVFSCSV | | | |
| | MHEALHNHYTQKSLSLS | | | |
| | PGK | | | |
| P9-30 | EVQLVESGGGLVQPGGS | DIQMTQSPSSLSASVGDRV | EVQLVESGGGL | DIQMTQSPSS |
| | LRLSCAASGFTFSTYTIH | TITCRASQSVSSAVAWYQQ | VQPGGSLRLSC | LSASVGDRV |
| | WVRQAPGKGLEWVAWI | KPGKAPKLLIYSASSLYSG | AASGFTFSTYTI | TITCRASQSV |
| | YPKGGSTDYADSVKGRF | VPSRFSGSRSGTDFTLTISS | HWVRQAPGKG | SSAVAWYQQ |
| | TISADTSKNTAYLQMNS | LQPEDFATYYCQQYLSSPY | LEWVAWIYPK | KPGKAPKLLI |
| | LRAEDTAVYYCARPSGY | TFGQGTKVEIKRTVAAPSV | GGSTDYADSVK | YSASSLYSGV |
| | GFDYWGQGTLVTVSSAS | FIFPPSDSQLKSGTASVVCL | GRFTISADTSKN | PSRFSGSRSG |
| | TKGPSVFPLAPSSKSTSG | LNNFYPREAKVQWKVDN | TAYLQMNSLRA | TDFTLTISSLQ |
| | GTAALGCLVKDYFPEPV | ALQSGNSQESVTEQDSKDS | EDTAVYYCARP | PEDFATYYC |
| | TVSWNSGALTSGVHTFP | TYSLSSTLTLSKADYEKHK | SGYGFDYWGQ | QQYLSSPYTF |
| | AVLQSSGLYSLSSVVTV | VYACEVTHQGLSSPVTKSF | GTLVTVSS | GQGTKVEIKR |
| | PSSSLGTQTYICNVNHKP | NRGEC | | TV |
| | SNTKVDKKVEPKSCDKT | | | |
| | HTCPPCPAPELLGGPSVF | | | |
| | LFPPKPKDTLMISRTPEV | | | |
| | TCVVVDVSHEDPEVKFN | | | |
| | WYVDGVEVHNAKTKPR | | | |
| | EEQYNSTYRVVSVLTVL | | | |
| | HQDWLNGKEYKCKVSN | | | |
| | KALPAPIEKTISKAKGQP | | | |
| | REPQVYTLPPSRDELTK | | | |
| | NQVSLTCLVKGFYPSDI | | | |
| | AVEWESNGQPENNYKT | | | |
| | TPPVLDSDGSFFLYSKLT | | | |
| | VDKSRWQQGNVFSCSV | | | |
| | MHEALHNHYTQKSQSLS | | | |
| | PGK | | | |
| P9-34 | EVQLVESGGGLVQPGGS | DIQMTQSPSSLSASVGDRV | EVQLVESGGGL | DIQMTQSPSS |
| | LRLSCAASGFTFSTYFIH | TITCRASQSVSSAVAWYQQ | VQPGGSLRLSC | LSASVGDRV |
| | WVRQAPGKGLEWVAYI | KPGKAPKLLIYSASSLYSG | AASGFTFSTYFI | TITCRASQSV |
| | YPQGGYTYYADSVKGR | VPSRFSGSRSGTDFTLTISS | HWVRQAPGKG | SSAVAWYQQ |
| | FTISADTSKNTAYLQMN | LQPEDFATYYCQQWTIALT | LEWVAYIYPQG | KPGKAPKLLI |
| | SLRAEDTAVYYCARQSY | TFGQGTKVEIKRTVAAPSV | GYTYYADSVK | YSASSLYSGV |
| | PGVFDYWGQGTLVTVSS | FIFPPSDSQLKSGTASVVCL | GRFTISADTSKN | PSRFSGSRSG |
| | ASTKGPSVFPLAPSSKST | LNNFYPREAKVQWKVDN | TAYLQMNSLRA | TDFTLTISSLQ |
| | SGGTAALGCLVKDYFPE | ALQSGNSQESVTEQDSKDS | EDTAVYYCAR | PEDFATYYC |
| | PVTVSWNSGALTSGVHT | TYSLSSTLTLSKADYEKHK | QSYPGVFDYW | QQWTIALTTF |
| | FPAVLQSSGLYSLSSVVT | VYACEVTHQGLSSPVTKSF | GQGTLVTVSS | GQGTKVEIKR |
| | VPSSSLGTQTYICNVNH | NRGEC | | TV |
| | KPSNTKVDKKVEPKSCD | | | |
| | KTHTCPPCPAPELLGGPS | | | |
| | VFLFPPKPKDTLMISRTP | | | |
| | EVTCVVVDVSHEDPEVK | | | |
| | FNWYVDGVEVHNAKTK | | | |
| | PREEQYNSTYRVVSVLT | | | |
| | VLHQDWLNGKEYKCKV | | | |
| | SNKALPAPIEKTISKAKG | | | |
| | QPREPQVYTLPPSRDELT | | | |
| | KNQVSLTCLVKGFYPSD | | | |
| | IAVEWESNGQPENNYKT | | | |
| | TPPVLDSDGSFFLYSKLT | | | |
| | VDKSRWQQGNVFSCSV | | | |
| | MHEALHNHYTQKSLSLS | | | |
| | PGK | | | |
| P9-37 | EVQLVESGGGLVQPGGS | DIQMTQSPSSLSASVGDRV | EVQLVESGGGL | DIQMTQSPSS |
| | LRLSCAASGFTFWKYGI | TITCRASQSVSSAVAWYQQ | VQPGGSLRLSC | LSASVGDRV |
| | HWVRQAPGKGLEWVA | KPGKAPKLLIYSASSLYSG | AASGFTFWKYG | TITCRASQSV |
| | YIYPAGGITSYADSVKG | VPSRFSGSRSGTDFTLTISS | IHWVRQAPGKG | SSAVAWYQQ |
| | RFTISADTSKNTAYLQM | LQPEDFATYYCQQYYPSPS | LEWVAYIYPAG | KPGKAPKLLI |
| | NSLRAEDTAVYYCARSD | TFGQGTKVEIKRTVAAPSV | GITSYADSVKG | YSASSLYSGV |
| | YYSGMGMDYWGQGTL | FIFPPSDSQLKSGTASVVCL | RFTISADTSKNT | PSRFSGSRSG |
| | VTVSSASTKGPSVFPLAP | LNNFYPREAKVQWKVDN | AYLQMNSLRAE | TDFTLTISSLQ |
| | SSKSTSGGTAALGCLVK | ALQSGNSQESVTEQDSKDS | DTAVYYCARSD | PEDFATYYC |
| | DYFPEPVTVSWNSGALT | TYSLSSTLTLSKADYEKHK | YYSGMGMDY | QQYYPSPSTF |
| | SGVHTFPAVLQSSGLYS | VYACEVTHQGLSSPVTKSF | WGQGTLVTVSS | GQGTKVEIKR |
| | LSSVVTVPSSSLGTQTYI | NRGEC | | TV |
| | CNVNHKPSNTKVDKKV | | | |
| | EPKSCDKTHTCPPCPAPE | | | |
| | LLGGPSVFLFPPKPKDTL | | | |
| | MISRTPEVTCVVVDVSH | | | |
| | EDPEVKFNWYVDGVEV | | | |
| | HNAKTKPREEQYNSTYR | | | |
| | VVSVLTVLHQDWLNGK | | | |
| | EYKCKVSNKALPAPIEK | | | |
| | TISKAKGQPREPQVYTLP | | | |
| | PSRDELTKNQVSLTCLV | | | |
| | KGFYPSDIAVEWESNGQ | | | |
| | PENNYKTTPPVLDSDGS | | | |
| | FFLYSKLTVDKSRWQQG | | | |
| | NVFSCSVMHEALHNHY | | | |
| | TQKSLSLSPGK | | | |
| P9-38 | EVQLVESGGGLVQPGGS | DIQMTQSPSSLSASVGDRV | EVQLVESGGGL | DIQMTQSPSS |
| | LRLSCAASGFTFSSYWIH | TITCRASQSVSSAVAWYQQ | VQPGGSLRLSC | LSASVGDRV |
| | WVRQAPGKGLEWVAWI | KPGKAPKLLIYSASSLYSG | AASGFTFSSYWI | TITCRASQSV |
| | DPDYGTTSYADSVKGRF | VPSRFSGSRSGTDFTLTISS | HWVRQAPGKG | SSAVAWYQQ |
| | TISADTSKNTAYLQMNS | LQPEDFATYYCQQGSYFLQ | LEWVAWIDPD | KPGKAPKLLI |
| | LRAEDTAVYYCARSETG | TFGQGTKVEIKRTVAAPSV | YGTTSYADSVK | YSASSLYSGV |
| | AAMDYWGQGTLVTVSS | FIFPPSDSQLKSGTASVVCL | GRFTISADTSKN | PSRFSGSRSG |
| | ASTKGPSVFPLAPSSKST | LNNFYPREAKVQWKVDN | TAYLQMNSLRA | TDFTLTISSLQ |
| | SGGTAALGCLVKDYFPE | ALQSGNSQESVTEQDSKDS | EDTAVYYCARS | PEDFATYYC |
| | PVTVSWNSGALTSGVHT | TYSLSSTLTLSKADYEKHK | ETGAAMDYWG | QQGSYFLQTF |
| | FPAVLQSSGLYSLSSVVT | VYACEVTHQGLSSPVTKSF | QGTLVTVSS | GQGTKVEIKR |
| | VPSSSLGTQTYICNVNH | NRGEC | | TV |
| | KPSNTKVDKKVEPKSCD | | | |
| | KTHTCPPCPAPELLGGPS | | | |
| | VFLFPPKPKDTLMISRTP | | | |
| | EVTCVVVDVSHEDPEVK | | | |
| | FNWYVDGVEVHNAKTK | | | |
| | PREEQYNSTYRVVSVLT | | | |
| | VLHQDWLNGKEYKCKV | | | |
| | SNKALPAPIEKTISKAKG | | | |
| | QPREPQVYTLPPSRDELT | | | |
| | KNQVSLTCLVKGFYPSD | | | |
| | IAVEWESNGQPENNYKT | | | |
| | TPPVLDSDGSFFLYSKLT | | | |
| | VDKSRWQQGNVFSCSV | | | |
| | MetHEALHNHYTQKSLS | | | |
| | LSPGK | | | |
| P9-40 | EVQLVESGGGLVQPGGS | DIQMTQSPSSLSASVGDRV | EVQLVESGGGL | DIQMTQSPSS |
| | LRLSCAASGFTFRWYYI | TITCRASQSVSSAVAWYQQ | VQPGGSLRLSC | LSASVGDRV |
| | HWVRQAPGKGLEWVAT | KPGKAPKLLIYSASSLYSG | AASGFTFRWYY | TITCRASQSV |
| | IYPDWDYTTYADSVKGR | VPSRFSGSRSGTDFTLTISS | IHWVRQAPGKG | SSAVAWYQQ |
| | FTISADTSKNTAYLQMN | LQPEDFATYYCQQPTYSL | LEWVATIYPDW | KPGKAPKLLI |
| | SLRAEDTAVYYCARSPV | WTFGQGTKVEIKRTVAAPS | DYTTYADSVKG | YSASSLYSGV |
| | TGPYGFDYWGQGTLVT | VFIFPPSDSQLKSGTASVVC | RFTISADTSKNT | PSRFSGSRSG |
| | VSSASTKGPSVFPLAPSS | LLNNFYPREAKVQWKVDN | AYLQMNSLRAE | TDFTLTISSLQ |
| | KSTSGGTAALGCLVKDY | ALQSGNSQESVTEQDSKDS | DTAVYYCARSP | PEDFATYYC |
| | FPEPVTVSWNSGALTSG | TYSLSSTLTLSKADYEKHK | VTGPYGFDYW | QQPTYSLWT |
| | VHTFPAVLQSSGLYSLSS | VYACEVTHQGLSSPVTKSF | GQGTLVTVSS | FGQGTKVEIK |
| | VVTVPSSSLGTQTYICNV | NRGEC | | RTV |
| | NHKPSNTKVDKKVEPKS | | | |
| | CDKTHTCPPCPAPELLG | | | |
| | GPSVFLFPPKPKDTLMIS | | | |
| | RTPEVTCVVVDVSHEDP | | | |
| | EVKFNWYVDGVEVHNA | | | |
| | KTKPREEQYNSTYRVVS | | | |
| | VLTVLHQDWLNGKEYK | | | |
| | CKVSNKALPAPIEKTISK | | | |
| | AKGQPREPQVYTLPPSR | | | |
| | DELTKNQVSLTCLVKGF | | | |
| | YPSDIAVEWESNGQPEN | | | |
| | NYKTTPPVLDSDGSFFL | | | |
| | YSKLTVDKSRWQQGNV | | | |
| | FSCSVMHEALHNHYTQ | | | |
| | KSLSLSPGK | | | |
| P9-41 | EVQLVESGGGLVQPGGS | DIQMTQSPSSLSASVGDRV | ′EVQLVESGGGL | DIQMTQSPSS |
| | LRLSCAASGFTFRYYWI | TITCRASQSVSSAVAWYQQ | VQPGGSLRLSC | LSASVGDRV |
| | HWVRQAPGKGLEWVA | KPGKAPKLLIYSASSLYSG | AASGFTFRYYW | TITCRASQSV |
| | AIYPSSDSTYYADSVKG | VPSRFSGSRSGTDFTLTISS | IHWVRQAPGKG | SSAVAWYQQ |
| | RFTISADTSKNTAYLQM | LQPEDFATYYCQQWYSSL | LEWVAAIYPSS | KPGKAPKLLI |
| | NSLRAEDTAVYYCARSS | WTFGQGTKVEIKRTVAAPS | DSTYYADSVKG | YSASSLYSGV |
| | PYPYGQGVFDYWGQGT | VFIFPPSDSQLKSGTASVVC | RFTISADTSKNT | PSRFSGSRSG |
| | LVTVSSASTKGPSVFPLA | LLNNFYPREAKVQWKVDN | AYLQMNSLRAE | TDFTLTISSLQ |
| | PSSKSTSGGTAALGCLV | ALQSGNSQESVTEQDSKDS | DTAVYYCARSS | PEDFATYYC |
| | KDYFPEPVTVSWNSGAL | TYSLSSTLTLSKADYEKHK | PYPYGQGVFDY | QQWYSSLWT |
| | TSGVHTFPAVLQSSGLY | VYACEVTHQGLSSPVTKSF | WGQGTLVTVSS | FGQGTKVEIK |
| | SLSSVVTVPSSSLGTQTY | NRGEC | | RTV |
| | ICNVNHKPSNTKVDKKV | | | |
| | EPKSCDKTHTCPPCPAPE | | | |
| | LLGGPSVFLFPPKPKDTL | | | |
| | MISRTPEVTCVVVDVSH | | | |
| | EDPEVKFNWYVDGVEV | | | |
| | HNAKTKPREEQYNSTYR | | | |
| | VVSVLTVLHQDWLNGK | | | |
| | EYKCKVSNKALPAPIEK | | | |
| | TISKAKGQPREPQVYTLP | | | |
| | PSRDELTKNQVSLTCLV | | | |
| | KGFYPSDIAVEWESNGQ | | | |
| | PENNYKTTPPVLDSDGS | | | |
| | FFLYSKLTVDKSRWQQG | | | |
| | NVFSCSVMHEALHNHY | | | |
| | TQKSLSLSPGK | | | |
| P9-42 | EVQLVESGGGLVQPGGS | DIQMTQSPSSLSASVGDRV | EVQLVESGGGL | DIQMTQSPSS |
| | LRLSCAASGFTFSSYYIH | TITCRASQSVSSAVAWYQQ | VQPGGSLRLSC | LSASVGDRV |
| | WVRQAPGKGLEWVAAI | KPGKAPKLLIYSASSLYSG | AASGFTFSSYYI | TITCRASQSV |
| | YSAWGTTYYADSVKGR | VPSRFSGSRSGTDFTLTISS | HWVRQAPGKG | SSAVAWYQQ |
| | FTISADTSKNTAYLQMN | LQPEDFATYYCQQWSSDL | LEWVAAIYSA | KPGKAPKLLI |
| | SLRAEDTAVYYCARSYG | VTFGQGTKVEIKRTVAAPS | WGTTYYADSV | YSASSLYSGV |
| | YVFGYYSGMDYWGQGT | VFIFPPSDSQLKSGTASVVC | KGRFTISADTSK | PSRFSGSRSG |
| | LVTVSSASTKGPSVFPLA | LLNNFYPREAKVQWKVDN | NTAYLQMNSLR | TDFTLTISSLQ |
| | PSSKSTSGGTAALGCLV | ALQSGNSQESVTEQDSKDS | AEDTAVYYCA | PEDFATYYC |
| | KDYFPEPVTVSWNSGAL | TYSLSSTLTLSKADYEKHK | RSYGYVFGYYS | QQWSSDLVT |
| | TSGVHTFPAVLQSSGLY | VYACEVTHQGLSSPVTKSF | GMDYWGQGTL | FGQGTKVEIK |
| | SLSSVVTVPSSSLGTQTY | NRGEC | VTVSS | RTV |
| | ICNVNHKPSNTKVDKKV | | | |
| | EPKSCDKTHTCPPCPAPE | | | |
| | LLGGPSVFLFPPKPKDTL | | | |
| | MISRTPEVTCVVVDVSH | | | |
| | EDPEVKFNWYVDGVEV | | | |
| | HNAKTKPREEQYNSTYR | | | |
| | VVSVLTVLHQDWLNGK | | | |
| | EYKCKVSNKALPAPIEK | | | |
| | TISKAKGQPREPQVYTLP | | | |
| | PSRDELTKNQVSLTCLV | | | |
| | KGFYPSDIAVEWESNGQ | | | |
| | PENNYKTTPPVLDSDGS | | | |
| | FFLYSKLTVDKSRWQQG | | | |
| | NVFSCSVMHEALHNHY | | | |
| | TQKSLSLSPGK | | | |
| P9-43 | EVQLVESGGGLVQPGGS | DIQMTQSPSSLSASVGDRV | EVQLVESGGGL | DIQMTQSPSS |
| | LRLSCAASGFTFHSYWI | TITCRASQSVSSAVAWYQQ | VQPGGSLRLSC | LSASVGDRV |
| | HWVRQAPGKGLEWVAR | KPGKAPKLLIYSASSLYSG | AASGFTFHSYW | TITCRASQSV |
| | IDSSKFGTYYADSVKGR | VPSRFSGSRSGTDFTLTISS | IHWVRQAPGKG | SSAVAWYQQ |
| | FTISADTSKNTAYLQMN | LQPEDFATYYCQQVYFSPY | LEWVARIDSSK | KPGKAPKLLI |
| | SLRAEDTAVYYCARSYI | TFGQGTKVEIKRTVAAPSV | FGTYYADSVKG | YSASSLYSGV |
| | DYPVSPAVFDYWGQGT | FIFPPSDSQLKSGTASVVCL | RFTISADTSKNT | PSRFSGSRSG |
| | LVTVSSASTKGPSVFPLA | LNNFYPREAKVQWKVDN | AYLQMNSLRAE | TDFTLTISSLQ |
| | PSSKSTSGGTAALGCLV | ALQSGNSQESVTEQDSKDS | DTAVYYCARSY | PEDFATYYC |
| | KDYFPEPVTVSWNSGAL | TYSLSSTLTLSKADYEKHK | IDYPVSPAVFD | QQVYFSPYTF |
| | TSGVHTFPAVLQSSGLY | VYACEVTHQGLSSPVTKSF | YWGQGTLVTV | GQGTKVEIKR |
| | SLSSVVTVPSSSLGTQTY | NRGEC | SS | TV |
| | ICNVNHKPSNTKVDKKV | | | |
| | EPKSCDKTHTCPPCPAPE | | | |
| | LLGGPSVFLFPPKPKDTL | | | |
| | MISRTPEVTCVVVDVSH | | | |
| | EDPEVKFNWYVDGVEV | | | |
| | HNAKTKPREEQYNSTYR | | | |
| | VVSVLTVLHQDWLNGK | | | |
| | EYKCKVSNKALPAPIEK | | | |
| | TISKAKGQPREPQVYTLP | | | |
| | PSRDELTKNQVSLTCLV | | | |
| | KGFYPSDIAVEWESNGQ | | | |
| | PENNYKTTPPVLDSDGS | | | |
| | FFLYSKLTVDKSRWQQG | | | |
| | NVFSCSVMHEALHNHY | | | |
| | TQKSLSLSPGK | | | |
| P9-44 | EVQLVESGGGLVQPGGS | DIQMTQSPSSLSASVGDRV | EVQLVESGGGL | DIQMTQSPSS |
| | LRLSCAASGFTFSYYWI | TITCRASQSVSSAVAWYQQ | VQPGGSLRLSC | LSASVGDRV |
| | HWVRQAPGKGLEWVA | KPGKAPKLLIYSASSLYSG | AASGFTFSYYW | TITCRASQSV |
| | AISPSGSYTSYADSVKGR | VPSRFSGSRSGTDFTLTISS | IHWVRQAPGKG | SSAVAWYQQ |
| | FTISADTSKNTAYLQMN | LQPEDFATYYCQQGIDSPE | LEWVAAISPSG | KPGKAPKLLI |
| | SLRAEDTAVYYCARSYY | TFGQGTKVEIKRTVAAPSV | SYTSYADSVKG | YSASSLYSGV |
| | RFRTPYTVMDYWGQGT | FIFPPSDSQLKSGTASVVCL | RFTISADTSKNT | PSRFSGSRSG |
| | LVTVSSASTKGPSVFPLA | LNNFYPREAKVQWKVDN | AYLQMNSLRAE | TDFTLTISSLQ |
| | PSSKSTSGGTAALGCLV | ALQSGNSQESVTEQDSKDS | DTAVYYCARSY | PEDFATYYC |
| | KDYFPEPVTVSWNSGAL | TYSLSSTLTLSKADYEKHK | YRFRTPYTVMD | QQGIDSPETF |
| | TSGVHTFPAVLQSSGLY | VYACEVTHQGLSSPVTKSF | YWGQGTLVTV | GQGTKVEIKR |
| | SLSSVVTVPSSSLGTQTY | NRGEC | SS | TV |
| | ICNVNHKPSNTKVDKKV | | | |
| | EPKSCDKTHTCPPCPAPE | | | |
| | LLGGPSVFLFPPKPKDTL | | | |
| | MISRTPEVTCVVVDVSH | | | |
| | EDPEVKFNWYVDGVEV | | | |
| | HNAKTKPREEQYNSTYR | | | |
| | VVSVLTVLHQDWLNGK | | | |
| | EYKCKVSNKALPAPIEK | | | |
| | TISKAKGQPREPQVYTLP | | | |
| | PSRDELTKNQVSLTCLV | | | |
| | KGFYPSDIAVEWESNGQ | | | |
| | PENNYKTTPPVLDSDGS | | | |
| | FFLYSKLTVDKSRWQQG | | | |
| | NVFSCSVMHEALHNHY | | | |
| | TQKSLSLSPGK | | | |
| P9-45 | EVQLVESGGGLVQPGGS | DIQMTQSPSSLSASVGDRV | EVQLVESGGGL | DIQMTQSPSS |
| | LRLSCAASGFTFFSYVIH | TITCRASQSVSSAVAWYQQ | VQPGGSLRLSC | LSASVGDRV |
| | WVRQAPGKGLEWVAAI | KPGKAPKLLIYSASSLYSG | AASGFTFFSYVI | TITCRASQSV |
| | YPYSGYTTYADSVKGRF | VPSRFSGSRSGTDFTLTISS | HWVRQAPGKG | SSAVAWYQQ |
| | TISADTSKNTAYLQMNS | LQPEDFATYYCQQGWDSL | LEWVAAIYPYS | KPGKAPKLLI |
| | LRAEDTAVYYCARTKY | VTFGQGTKVEIKRTVAAPS | GYTTYADSVKG | YSASSLYSGV |
| | YDYHVFDYWGQGTLVT | VFIFPPSDSQLKSGTASVVC | RFTISADTSKNT | PSRFSGSRSG |
| | VSSASTKGPSVFPLAPSS | LLNNFYPREAKVQWKVDN | AYLQMNSLRAE | TDFTLTISSLQ |
| | KSTSGGTAALGCLVKDY | ALQSGNSQESVTEQDSKDS | DTAVYYCART | PEDFATYYC |
| | FPEPVTVSWNSGALTSG | TYSLSSTLTLSKADYEKHK | KYYDYHVFDY | QQGWDSLVT |
| | VHTFPAVLQSSGLYSLSS | VYACEVTHQGLSSPVTKSF | WGQGTLVTVSS | FGQGTKVEIK |
| | VVTVPSSSLGTQTYICNV | NRGEC | | RTV |
| | NHKPSNTKVDKKVEPKS | | | |
| | CDKTHTCPPCPAPELLG | | | |
| | GPSVFLFPPKPKDTLMIS | | | |
| | RTPEVTCVVVDVSHEDP | | | |
| | EVKFNWYVDGVEVHNA | | | |
| | KTKPREEQYNSTYRVVS | | | |
| | VLTVLHQDWLNGKEYK | | | |
| | CKVSNKALPAPIEKTISK | | | |
| | AKGQPREPQVYTLPPSR | | | |
| | DELTKNQVSLTCLVKGF | | | |
| | YPSDIAVEWESNGQPEN | | | |
| | NYKTTPPVLDSDGSFFL | | | |
| | YSKLTVDKSRWQQGNV | | | |
| | FSCSVMHEALHNHYTQ | | | |
| | KSLSLSPGK | | | |
| P9-46 | EVQLVESGGGLVQPGGS | DIQMTQSPSSLSASVGDRV | EVQLVESGGGL | DIQMTQSPSS |
| | LRLSCAASGFTFSRYYIH | TITCRASQSVSSAVAWYQQ | VQPGGSLRLSC | LSASVGDRV |
| | WVRQAPGKGLEWVAFI | KPGKAPKLLIYSASSLYSG | AASGFTFSRYYI | TITCRASQSV |
| | SSDSGYTQYADSVKGRF | VPSRFSGSRSGTDFTLTISS | HWVRQAPGKG | SSAVAWYQQ |
| | TISADTSKNTAYLQMNS | LQPEDFATYYCQQYWWSP | LEWVAFISSDS | KPGKAPKLLI |
| | LRAEDTAVYYCARTMS | ETFGQGTKVEIKRTVAAPS | GYTQYADSVK | YSASSLYSGV |
| | YSALDYWGQGTLVTVS | VFIFPPSDSQLKSGTASVVC | GRFTISADTSKN | PSRFSGSRSG |
| | SASTKGPSVFPLAPSSKS | LLNNFYPREAKVQWKVDN | TAYLQMNSLRA | TDFTLTISSLQ |
| | TSGGTAALGCLVKDYFP | ALQSGNSQESVTEQDSKDS | EDTAVYYCART | PEDFATYYC |
| | EPVTVSWNSGALTSGVH | TYSLSSTLTLSKADYEKHK | MSYSALDYWG | QQYWWSPET |
| | TFPAVLQSSGLYSLSSVV | VYACEVTHQGLSSPVTKSF | QGTLVTVSS | FGQGTKVEIK |
| | TVPSSSLGTQTYICNVNH | NRGEC | | RTV |
| | KPSNTKVDKKVEPKSCD | | | |
| | KTHTCPPCPAPELLGGPS | | | |
| | VFLFPPKPKDTLMISRTP | | | |
| | EVTCVVVDVSHEDPEVK | | | |
| | FNWYVDGVEVHNAKTK | | | |
| | PREEQYNSTYRVVSVLT | | | |
| | VLHQDWLNGKEYKCKV | | | |
| | SNKALPAPIEKTISKAKG | | | |
| | QPREPQVYTLPPSRDELT | | | |
| | KNQVSLTCLVKGFYPSD | | | |
| | IAVEWESNGQPENNYKT | | | |
| | TPPVLDSDGSFFLYSKLT | | | |
| | VDKSRWQQGNVFSCSV | | | |
| | MHEALHNHYTQKSLSLS | | | |
| | PGK | | | |
| P9-50 | EVQLVESGGGLVQPGGS | DIQMTQSPSSLSASVGDRV | EVQLVESGGGL | DIQMTQSPSS |
| | LRLSCAASGFTFSSYVIH | TITCRASQSVSSAVAWYQQ | VQPGGSLRLSC | LSASVGDRV |
| | WVRQAPGKGLEWVALI | KPGKAPKLLIYSASSLYSG | AASGFTFSSYVI | TITCRASQSV |
| | YSSGGYTQYADSVKGRF | VPSRFSGSRSGTDFTLTISS | HWVRQAPGKG | SSAVAWYQQ |
| | TISADTSKNTAYLQMNS | LQPEDFATYYCQQFGSSLP | LEWVALIYSSG | KPGKAPKLLI |
| | LRAEDTAVYYCARVGT | TFGQGTKVEIKRTVAAPSV | GYTQYADSVK | YSASSLYSGV |
| | TYPSRYLEALDYWGQG | FIFPPSDSQLKSGTASVVCL | GRFTISADTSKN | PSRFSGSRSG |
| | TLVTVSSASTKGPSVFPL | LNNFYPREAKVQWKVDN | TAYLQMNSLRA | TDFTLTISSLQ |
| | APSSKSTSGGTAALGCL | ALQSGNSQESVTEQDSKDS | EDTAVYYCAR | PEDFATYYC |
| | VKDYFPEPVTVSWNSGA | TYSLSSTLTLSKADYEKHK | VGTTYPSRYLE | QQFGSSLPTF |
| | LTSGVHTFPAVLQSSGL | VYACEVTHQGLSSPVTKSF | ALDYWGQGTL | GQGTKVEIKR |
| | YSLSSVVTVPSSSLGTQT | NRGEC | VTVSS | TV |
| | YICNVNHKPSNTKVDKK | | | |
| | VEPKSCDKTHTCPPCPAP | | | |
| | ELLGGPSVFLFPPKPKDT | | | |
| | LMISRTPEVTCVVVDVS | | | |
| | HEDPEVKFNWYVDGVE | | | |
| | VHNAKTKPREEQYNSTY | | | |
| | RVVSVLTVLHQDWLNG | | | |
| | KEYKCKVSNKALPAPIE | | | |
| | KTISKAKGQPREPQVYT | | | |
| | LPPSRDELTKNQVSLTCL | | | |
| | VKGFYPSDIAVEWESNG | | | |
| | QPENNYKTTPPVLDSDG | | | |
| | SFFLYSKLTVDKSRWQQ | | | |
| | GNVFSCSVMHEALHNH | | | |
| | YTQKSLSLSPG | | | |
| P9-51 | EVQLVESGGGLVQPGGS | DIQMTQSPSSLSASVGDRV | EVQLVESGGGL | DIQMTQSPSS |
| | LRLSCAASGFTFSSYYIH | TITCRASQSVSSAVAWYQQ | VQPGGSLRLSC | LSASVGDRV |
| | WVRQAPGKGLEWVAGI | KPGKAPKLLIYSASSLYSG | AASGFTFSSYYI | TITCRASQSV |
| | YPEGSYTYYADSVKGRF | VPSRFSGSRSGTDFTLTISS | HWVRQAPGKG | SSAVAWYQQ |
| | TISADTSKNTAYLQMNS | LQPEDFATYYCQQWGSSL | LEWVAGIYPEG | KPGKAPKLLI |
| | LRAEDTAVYYCARVGY | ATFGQGTKVEIKRTVAAPS | SYTYYADSVKG | YSASSLYSGV |
| | PGVMDYWGQGTLVTVS | VFIFPPSDSQLKSGTASVVC | RFTISADTSKNT | PSRFSGSRSG |
| | SASTKGPSVFPLAPSSKS | LLNNFYPREAKVQWKVDN | AYLQMNSLRAE | TDFTLTISSLQ |
| | TSGGTAALGCLVKDYFP | ALQSGNSQESVTEQDSKDS | DTAVYYCARV | PEDFATYYC |
| | EPVTVSWNSGALTSGVH | TYSLSSTLTLSKADYEKHK | GYPGVMDYWG | QQWGSSLAT |
| | TFPAVLQSSGLYSLSSVV | VYACEVTHQGLSSPVTKSF | QGTLVTVSS | FGQGTKVEIK |
| | TVPSSSLGTQTYICNVNH | NRGEC | | RTV |
| | KPSNTKVDKKVEPKSCD | | | |
| | KTHTCPPCPAPELLGGPS | | | |
| | VFLFPPKPKDTLMISRTP | | | |
| | EVTCVVVDVSHEDPEVK | | | |
| | FNWYVDGVEVHNAKTK | | | |
| | PREEQYNSTYRVVSVLT | | | |
| | VLHQDWLNGKEYKCKV | | | |
| | SNKALPAPIEKTISKAKG | | | |
| | QPREPQVYTLPPSRDELT | | | |
| | KNQVSLTCLVKGFYPSD | | | |
| | IAVEWESNGQPENNYKT | | | |
| | TPPVLDSDGSFFLYSKLT | | | |
| | VDKSRWQQGNVFSCSV | | | |
| | MHEALHNHYTQKSLSLS | | | |
| | PGK | | | |
| P9-52 | EVQLVESGGGLVQPGGS | DIQMTQSPSSLSASVGDRV | EVQLVESGGGL | DIQMTQSPSS |
| | LRLSCAASGFTFSTYLIH | TITCRASQSVSSAVAWYQQ | VQPGGSLRLSC | LSASVGDRV |
| | WVRQAPGKGLEWVAAI | KPGKAPKLLIYSASSLYSG | AASGFTFSTYLI | TITCRASQSV |
| | TPYSGYTSYADSVKGRF | VPSRFSGSRSGTDFTLTISS | HWVRQAPGKG | SSAVAWYQQ |
| | TISADTSKNTAYLQMNS | LQPEDFATYYCQQLDYSL | LEWVAAITPYS | KPGKAPKLLI |
| | LRAEDTAVYYCARVGY | ATFGQGTKVEIKRTVAAPS | GYTSYADSVKG | YSASSLYSGV |
| | PMVMDYWGQGTLVTVS | VFIFPPSDSQLKSGTASVVC | RFTISADTSKNT | PSRFSGSRSG |
| | SASTKGPSVFPLAPSSKS | LLNNFYPREAKVQWKVDN | AYLQMNSLRAE | TDFTLTISSLQ |
| | TSGGTAALGCLVKDYFP | ALQSGNSQESVTEQDSKDS | DTAVYYCARV | PEDFATYYC |
| | EPVTVSWNSGALTSGVH | TYSLSSTLTLSKADYEKHK | GYPMVMDYW | QQLDYSLAT |
| | TFPAVLQSSGLYSLSSVV | VYACEVTHQGLSSPVTKSF | GQGTLVTVSS | FGQGTKVEIK |
| | TVPSSSLGTQTYICNVNH | NRGEC | | RTV |
| | KPSNTKVDKKVEPKSCD | | | |
| | KTHTCPPCPAPELLGGPS | | | |
| | VFLFPPKPKDTLMISRTP | | | |
| | EVTCVVVDVSHEDPEVK | | | |
| | FNWYVDGVEVHNAKTK | | | |
| | PREEQYNSTYRVVSVLT | | | |
| | VLHQDWLNGKEYKCKV | | | |
| | SNKALPAPIEKTISKAKG | | | |
| | QPREPQVYTLPPSRDELT | | | |
| | KNQVSLTCLVKGFYPSD | | | |
| | IAVEWESNGQPENNYKT | | | |
| | TPPVLDSDGSFFLYSKLT | | | |
| | VDKSRWQQGNVFSCSV | | | |
| | MHEALHNHYTQKSLSLS | | | |
| | PGK | | | |
| P9-53 | EVQLVESGGGLVQPGGS | DIQMTQSPSSLSASVGDRV | EVQLVESGGGL | DIQMTQSPSS |
| | LRLSCAASGFTFSRYQIH | TITCRASQSVSSAVAWYQQ | VQPGGSLRLSC | LSASVGDRV |
| | WVRQAPGKGLEWVAYI | KPGKAPKLLIYSASSLYSG | AASGFTFSRYQI | TITCRASQSV |
| | ASASGTTSYADSVKGRF | VPSRFSGSRSGTDFTLTISS | HWVRQAPGKG | SSAVAWYQQ |
| | TISADTSKNTAYLQMNS | LQPEDFATYYCQQGYPHP | LEWVAYIASAS | KPGKAPKLLI |
| | LRAEDTAVYYCARVPY | GTFGQGTKVEIKRTVAAPS | GTTSYADSVKG | YSASSLYSGV |
| | VAMDYWGQGTLVTVSS | VFIFPPSDSQLKSGTASVVC | RFTISADTSKNT | PSRFSGSRSG |
| | ASTKGPSVFPLAPSSKST | LLNNFYPREAKVQWKVDN | AYLQMNSLRAE | TDFTLTISSLQ |
| | SGGTAALGCLVKDYFPE | ALQSGNSQESVTEQDSKDS | DTAVYYCARVP | PEDFATYYC |
| | PVTVSWNSGALTSGVHT | TYSLSSTLTLSKADYEKHK | YVAMDYWGQ | QQGYPHPGT |
| | FPAVLQSSGLYSLSSVVT | VYACEVTHQGLSSPVTKSF | GTLVTVSS | FGQGTKVEIK |
| | VPSSSLGTQTYICNVNH | NRGEC | | RTV |
| | KPSNTKVDKKVEPKSCD | | | |
| | KTHTCPPCPAPELLGGPS | | | |
| | VFLFPPKPKDTLMISRTP | | | |
| | EVTCVVVDVSHEDPEVK | | | |
| | FNWYVDGVEVHNAKTK | | | |
| | PREEQYNSTYRVVSVLT | | | |
| | VLHQDWLNGKEYKCKV | | | |
| | SNKALPAPIEKTISKAKG | | | |
| | QPREPQVYTLPPSRDELT | | | |
| | KNQVSLTCLVKGFYPSD | | | |
| | IAVEWESNGQPENNYKT | | | |
| | TPPVLDSDGSFFLYSKLT | | | |
| | VDKSRWQQGNVFSCSV | | | |
| | MHEALHNHYTQKSLSLS | | | |
| | PG | | | |
| P9-55 | EVQLVESGGGLVQPGGS | DIQMTQSPSSLSASVGDRV | EVQLVESGGGL | DIQMTQSPSS |
| NEG. | LRLSCAASGFTFATYYIH | TITCRASQSVSSAVAWYQQ | VQPGGSLRLSC | LSASVGDRV |
| CON. | WVRQAPGKGLEWVAYI | KPGKAPKLLIYSASSLYSG | AASGFTFATYYI | TITCRASQSV |
| | DSESGYTYYADSVKGRF | VPSRFSGSRSGTDFTLTISS | HWVRQAPGKG | SSAVAWYQQ |
| | TISADTSKNTAYLQMNS | LQPEDFATYYCQQRYSSLL | LEWVAYIDSES | KPGKAPKLLI |
| | LRAEDTAVYYCARVSR | TFGQGTKVEIKRTVAAPSV | GYTYYADSVK | YSASSLYSGV |
| | GSSGTHVMDYWGQGTL | FIFPPSDSQLKSGTASVVCL | GRFTISADTSKN | PSRFSGSRSG |
| | VTVSSASTKGPSVFPLAP | LNNFYPREAKVQWKVDN | TAYLQMNSLRA | TDFTLTISSLQ |
| | SSKSTSGGTAALGCLVK | ALQSGNSQESVTEQDSKDS | EDTAVYYCAR | PEDFATYYC |
| | DYFPEPVTVSWNSGALT | TYSLSSTLTLSKADYEKHK | VSRGSSGTHVM | QQRYSSLLTF |
| | SGVHTFPAVLQSSGLYS | VYACEVTHQGLSSPVTKSF | DYWGQGTLVT | GQGTKVEIKR |
| | LSSVVTVPSSSLGTQTYI | NRGEC | VSS | TV |
| | CNVNHKPSNTKVDKKV | | | |
| | EPKSCDKTHTCPPCPAPE | | | |
| | LLGGPSVFLFPPKPKDTL | | | |
| | MISRTPEVTCVVVDVSH | | | |
| | EDPEVKFNWYVDGVEV | | | |
| | HNAKTKPREEQYNSTYR | | | |
| | VVSVLTVLHQDWLNGK | | | |
| | EYKCKVSNKALPAPIEK | | | |
| | TISKAKGQPREPQVYTLP | | | |
| | PSRDELTKNQVSLTCLV | | | |
| | KGFYPSDIAVEWESNGQ | | | |
| | PENNYKTTPPVLDSDGS | | | |
| | FFLYSKLTVDKSRWQQG | | | |
| | NVFSCSVMHEALHNHY | | | |
| | TQKSLSLSPGK | | | |
| P9-56 | EVQLVESGGGLVQPGGS | DIQMTQSPSSLSASVGDRV | EVQLVESGGGL | DIQMTQSPSS |
| | LRLSCAASGFTFSSYYIH | TITCRASQSVSSAVAWYQQ | VQPGGSLRLSC | LSASVGDRV |
| | WVRQAPGKGLEWVAYI | KPGKAPKLLIYSASSLYSG | AASGFTFSSYYI | TITCRASQSV |
| | DSSGKYTDYADSVKGRF | VPSRFSGSRSGTDFTLTISS | HWVRQAPGKG | SSAVAWYQQ |
| | TISADTSKNTAYLQMNS | LQPEDFATYYCQQYDYSL | LEWVAYIDSSG | KPGKAPKLLI |
| | LRAEDTAVYYCARYAY | WTFGQGTKVEIKRTVAAPS | KYTDYADSVK | YSASSLYSGV |
| | PGVMDYWGQGTLVTVS | VFIFPPSDSQLKSGTASVVC | GRFTISADTSKN | PSRFSGSRSG |
| | SASTKGPSVFPLAPSSKS | LLNNFYPREAKVQWKVDN | TAYLQMNSLRA | TDFTLTISSLQ |
| | TSGGTAALGCLVKDYFP | ALQSGNSQESVTEQDSKDS | EDTAVYYCAR | PEDFATYYC |
| | EPVTVSWNSGALTSGVH | TYSLSSTLTLSKADYEKHK | YAYPGVMDYW | QQYDYSLWT |
| | TFPAVLQSSGLYSLSSVV | VYACEVTHQGLSSPVTKSF | GQGTLVTVSS | FGQGTKVEIK |
| | TVPSSSLGTQTYICNVNH | NRGEC | | RTV |
| | KPSNTKVDKKVEPKSCD | | | |
| | KTHTCPPCPAPELLGGPS | | | |
| | VFLFPPKPKDTLMISRTP | | | |
| | EVTCVVVDVSHEDPEVK | | | |
| | FNWYVDGVEVHNAKTK | | | |
| | PREEQYNSTYRVVSVLT | | | |
| | VLHQDWLNGKEYKCKV | | | |
| | SNKALPAPIEKTISKAKG | | | |
| | QPREPQVYTLPPSRDELT | | | |
| | KNQVSLTCLVKGFYPSD | | | |
| | IAVEWESNGQPENNYKT | | | |
| | TPPVLDRDGSFFLYSKLT | | | |
| | VDKSRWQQGNVFSCSV | | | |
| | MHEALHNHYTQKSLSLS | | | |
| | PGK | | | |
| P9-57 | EVQLVESGGGLVQPGGS | DIQMTQSPSSLSASVGDRV | EVQLVESGGGL | DIQMTQSPSS |
| | LRLSCAASGFTFSSYYIH | TITCRASQSVSSAVAWYQQ | VQPGGSLRLSC | LSASVGDRV |
| | WVRQAPGKGLEWVATI | KPGKAPKLLIYSASSLYSG | AASGFTFSSYYI | TITCRASQSV |
| | YPSGGYTYYADSVKGRF | VPSRFSGSRSGTDFTLTISS | HWVRQAPGKG | SSAVAWYQQ |
| | TISADTSKNTAYLQMNS | LQPEDFATYYCQQSSSFLW | LEWVATIYPSG | KPGKAPKLLI |
| | LRAEDTAVYYCARYSYP | TFGQGTKVEIKRTVAAPSV | GYTYYADSVK | YSASSLYSGV |
| | GVLDYWGQGTLVTVSS | FIFPPSDSQLKSGTASVVCL | GRFTISADTSKN | PSRFSGSRSG |
| | ASTKGPSVFPLAPSSKST | LNNFYPREAKVQWKVDN | TAYLQMNSLRA | TDFTLTISSLQ |
| | SGGTAALGCLVKDYFPE | ALQSGNSQESVTEQDSKDS | EDTAVYYCAR | PEDFATYYC |
| | PVTVSWNSGALTSGVHT | TYSLSSTLTLSKADYEKHK | YSYPGVLDYW | QQSSSFLWTF |
| | FPAVLQSSGLYSLSSVVT | VYACEVTHQGLSSPVTKSF | GQGTLVTVSS | GQGTKVEIKR |
| | VPSSSLGTQTYICNVNH | NRGEC | | TV |
| | KPSNTKVDKKVEPKSCD | | | |
| | KTHTCPPCPAPELLGGPS | | | |
| | VFLFPPKPKDTLMISRTP | | | |
| | EVTCVVVDVSHEDPEVK | | | |
| | FNWYVDGVEVHNAKTK | | | |
| | PREEQYNSTYRVVSVLT | | | |
| | VLHQDWLNGKEYKCKV | | | |
| | SNKALPAPIEKTISKAKG | | | |
| | QPREPQVYTLPPSRDELT | | | |
| | KNQVSLTCLVKGFYPSD | | | |
| | IAVEWESNGQPENNYKT | | | |
| | TPPVLDSDGSFFLYSKLT | | | |
| | VDKSRWQQGNVFSCSV | | | |
| | MHEALHNHYTQKSLSLS | | | |
| | PGK | | | |

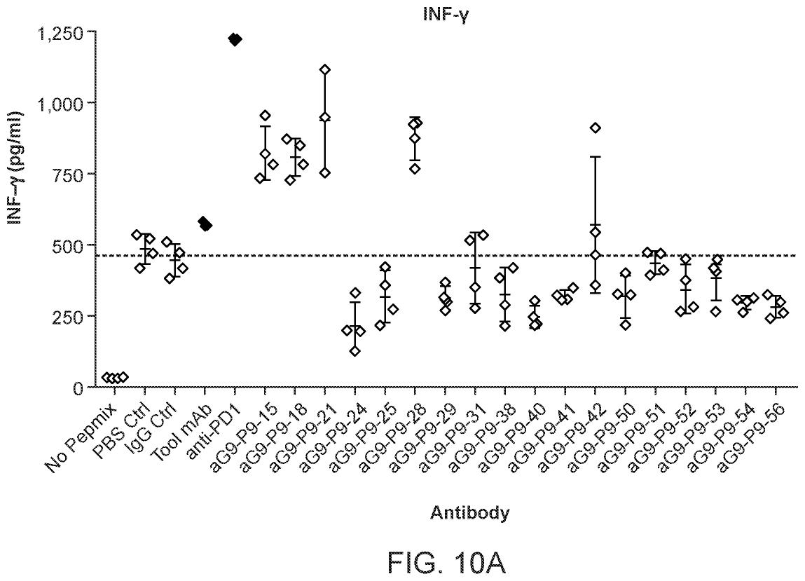

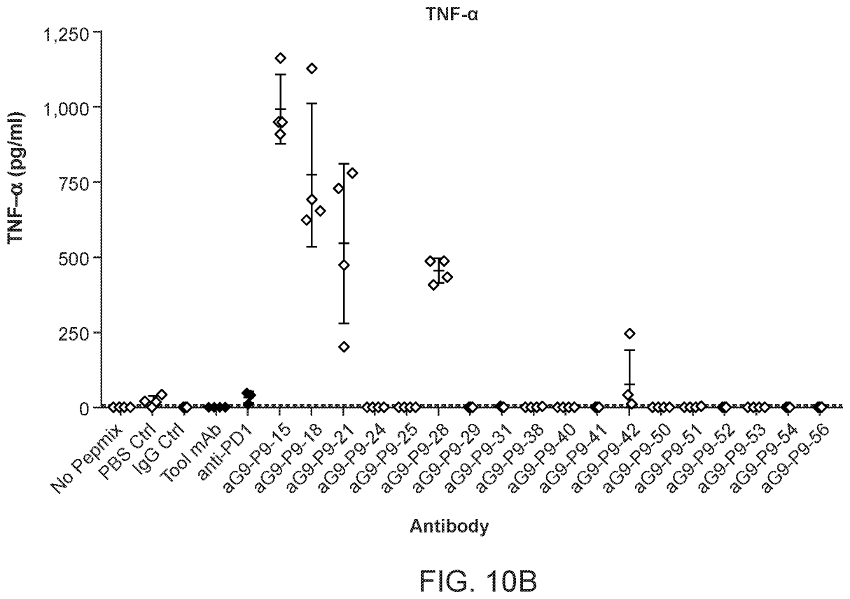

Select GAL9 binding candidates were analyzed for binding properties: cross-reactive binding with murine GAL9; qualitative binding; epitope binning (Bin 2—candidates bin with Commercial antibody Clone ECA8 from LS Bio [LS-C179448]; Bin 3—candidates Bins with Commercial antibody Clone ECA42 from LS Bio [LS-C179449], which is the “tool antibody” referenced in FIG. 10), and monovalent affinity binding. Analysis results are presented in Table 7.

TABLE 7
Candidate anti-human GAL9 Binding Properties

| | Mouse | Binding Off-Rate | | |
|---|---|---|---|---|
| | Cross- | (⁺⁺ = moderate; | | Calculated |
| ABS | reactivity | ⁺⁺⁺ = slow) | Bin | K_{D} (M) |
| P9-01 | Y | ⁺⁺⁺ | 1 | |
| P9-02A | Y | ⁺⁺⁺ | 1 | |
| P9-03 | | ⁺⁺⁺ | 1 | |
| P9-06 | | ⁺⁺ | 1 | |
| P9-07 | Y | ⁺⁺ | 3 | |
| P9-11 | Y | ⁺⁺⁺ | 1 | 6.554 × 10⁻⁹ |
| P9-12 | | ⁺⁺ | 3 | |
| P9-14 | | ⁺⁺⁺ | 2 | |
| P9-24 | | ⁺⁺⁺ | 1 | 5.409 × 10⁻⁹ |
| P9-25 | | ⁺⁺⁺ | 1 | 3.48 × 10⁻⁹ |
| P9-26 | | Negative Control (NC) | | |
| P9-29 | | ⁺⁺⁺ | 1 | |
| P9-30 | | ⁺⁺⁺ | 1 | |
| P9-34 | | ⁺⁺⁺ | 1 | |
| P9-37 | Y | ⁺⁺⁺ | 1 | 4.543 × 10⁻⁹ |
| P9-38 | | ⁺⁺ | 1 | |
| P9-40 | Y | ⁺⁺⁺ | 1 | |
| P9-41 | | ⁺⁺ | 1 | |
| P9-42 | Y | ⁺⁺ | 1 | |
| P9-43 | Y | ⁺⁺⁺ | 1 | |
| P9-45 | | ⁺⁺ | 3 | |
| P9-46 | | ⁺⁺⁺ | 2 | |
| P9-50 | Y | ⁺⁺⁺ | 3 | 1.206 × 10⁻⁹ |
| P9-51 | | ⁺⁺⁺ | 1 | |
| P9-52 | | ⁺⁺⁺ | 1 | |
| P9-53 | | ⁺⁺⁺ | 1 | |
| P9-55 | | Negative Control (NC) | | |
| P9-56 | Y | ⁺⁺⁺ | 1 | |
| P9-57 | Y | ⁺⁺⁺ | 1 | 2.557 × 10⁻⁹ |

Select GALS binding candidates were further analyzed for sequence motifs that could adversely affect antibody properties that are relevant to clinical development, such as stability, mutability, and immunogenicity. Computational analysis was performed according to Kumar and Singh (Developability of biotherapeutics: computational approaches. Boca Raton: CRC Press, Taylor & Francis Group, 2016). Analysis results are presented in Table 8, and demonstrate a limited number of adverse sequence motifs are present in the listed clones, indicating the potential for further clinical development.

TABLE 8
Candidate anti-human GAL9 Antibody Properties

| | CDR3 | | | | Number | Number | Number | Number | | Number | Number |
|---|---|---|---|---|---|---|---|---|---|---|---|
| | Loop | Yield | Mol Weight | Isoelectric | Deamidation | Isomerization | Fragmentation | N-linked | Cys in | Other | T-cell |
| ABS | Length | (ug/mL) | (kDa) | Point | Sites¹ | Sites² | Sites³ | Glycosylation Sites⁴ | CDR | Sites⁵ | Epitopes⁶ |
| P9-07 | 15 | 45 | 1.453 × 10⁵ | 8.08 | 0 | 3 | 1 | 0 | No | 0 | 1 |
| P9-11 | 14 | 68.85 | 1.446 × 10⁵ | 8.42 | 0 | 1 | 1 | 0 | No | 0 | 2 |
| P9-24 | 12 | 72.15 | 1.438 × 10⁵ | 8.43 | 0 | 2 | 2 | 0 | No | 0 | 0 |
| P9-25 | 12 | 163.5 | 1.444 × 10⁵ | 8.32 | 0 | 1 | 1 | 0 | No | 0 | 0 |
| P9-37 | 14 | 108.45 | 1.447 × 10⁵ | 8.42 | 0 | 1 | 2 | 0 | No | 0 | 1 |
| P9-50 | 18 | 78.6 | 1.453 × 10⁵ | 8.22 | 0 | 1 | 1 | 0 | No | 0 | 0 |
| P9-55 | — | — | 1.452 × 10⁵ | 8.42 | 0 | 2 | 1 | 0 | No | 0 | 0 |
| P9-57 | 12 | 30 | 1.442 × 10⁵ | 8.42 | 0 | 1 | 1 | 0 | No | 0 | 0 |

¹(NG, NS, NA, NH, ND)
²(DG, DP, DS)
³(DP, DY, HS, KT, HXS, SXH)
⁴(NXS/T)
⁵(LLQG, HPQ, FHENSP, LPRWG, HHH)
⁶3% in at least 2 of DRB1_0101, DRB1_0301, DRB1_0401, DRB1_0701, DRB1_1101, DRB1_1301, DRB1_1501, DRB1_0801

### 6.11.9. Example 8: Anti-Human GAL9 Candidates' Effect on Cytokine Production in Peripheral Blood Mononuclear Cells (PBMCs)

Candidate anti-human GAL9 antigen binding sites (ABSs) were formatted into a bivalent monospecific native human full-length IgG1 heavy chain and light chain architecture (SEQ ID NO:5 and SEQ ID NO:3, respectively) and were tested for their effect on cytokine production by human PBMCs following peptide stimulation. PBMCs were stimulated essentially as described in Section 6.11.1 above. Briefly, PBMCs were harvested from human donors known to be responsive to human CMV virus (HCMV) placed in culture, and stimulated with HCMV PepMix to prime an antigen specific response, and treated with one of: control IgG, a comparator anti-human GAL9 tool activating mAb (clone ECA42, murine IgG2a), α-PD1 (Nivolumab), or candidate anti-GAL9 antibodies formatted as bivalent monospecific full-length human IgG1 antibodies. Cytokine secretion was measured at 24 and 72 hrs post-treatment by bead cytokine array. Results for INF-γ and TNF-α are depicted in FIGS. 10A and 10B. The data shown in FIG. 10 is described in more detail in Table 9 and Table 10 provided below.

TABLE 9
INF-γ 72 hr

| | | Average/donor | | | | |
|---|---|---|---|---|---|---|
| | | Donor 19 | Donor 25 | Donor 27 | Average | as % |
| IgG | pg/ml | 5922 | 43775 | 1657 | | |
| P9-11 | pg/ml | 5891 | 22998 | 891 | | |
| | Fold change | 0.99 | 0.52 | 0.53 | 0.68 | 68.2 |
| P9-24 | pg/ml | NT | 35748 | 1258 | | |
| | Fold change | | 0.82 | 0.78 | 0.80 | 87.6 |
| P9-34 | pg/ml | NT | 44378 | 1048 | | |
| | Fold change | | 1.01 | 0.74 | 0.88 | 87.6 |
| P9-37 | pg/ml | 3231 | NT | NT | | |
| | Fold change | 0.55 | | | 0.55 | 54.56 |
| P9-57 | pg/ml | 4939 | NT | NT | | |
| | Fold change | 0.83 | | | 0.83 | 83.4 |

TABLE 10
TNF-α 72 hr

| | | Average/donor | | | | |
|---|---|---|---|---|---|---|
| | | Donor 19 | Donor 25 | Donor 27 | Average | as % |
| IgG | pg/ml | 777 | 1284 | 929 | | |
| P9-11 | pg/ml | 607 | 982 | 374 | | |
| | Fold change | 0.78 | 0.76 | 0.40 | 0.64 | 64.7 |
| P9-24 | pg/ml | NT | 962 | 299 | | |
| | Fold change | | 0.75 | 0.32 | 0.54 | 53.5 |
| P9-34 | pg/ml | NT | 874 | 596 | | |
| | Fold change | | 0.68 | 0.79 | 0.74 | 73.7 |
| P9-37 | pg/ml | 429 | NT | NT | | |
| | Fold change | 0.55 | | | 0.55 | 55.2 |
| P9-57 | pg/ml | 417 | NT | NT | | |
| | Fold change | 0.54 | | | 0.54 | 53.66 |

### 6.11.10. Example 9: Treating with Anti-Human GAL9 IgG1 Antibodies P9-11, P9-37, or P9-57 Decreases Production of TNF-α and IFN-γ in Activated PBMCs

Selected inhibitory anti-human GAL9 candidates from Example 7, formatted as bivalent monospecific human IgG1 antibodies, were further tested on PBMCs from three additional human donors for their ability to inhibit cytokine production in PBMCs.

Stimulation of PBMCs

Human primary PBMC were collected from donor 19, donor RCB, and donor RG, which are known to have strong responses to human CMV virus (HCMV). PBMCs were stimulated essentially as described in Section 6.11.1 above. Briefly, PBMCs were harvested from human donors known to be responsive to human CMV virus (HCMV), placed in culture, stimulated with HCMV PepMix to prime an antigen specific response, and treated with P9-41, P9-42, P9-53, P9-11, P9-37, or P9-57, formatted as bivalent monospecific full length human IgG1 antibodies, or a human IgG control.

Cytokine Assay

Secretion of TNF-α and IFN-γ was measured at 24 hrs and 72 hrs post-treatment using BD™ Cytometric Bead Array (CBA) following the manufacturer's instructions. Assays were performed in quadruplicate.

Results/Conclusion

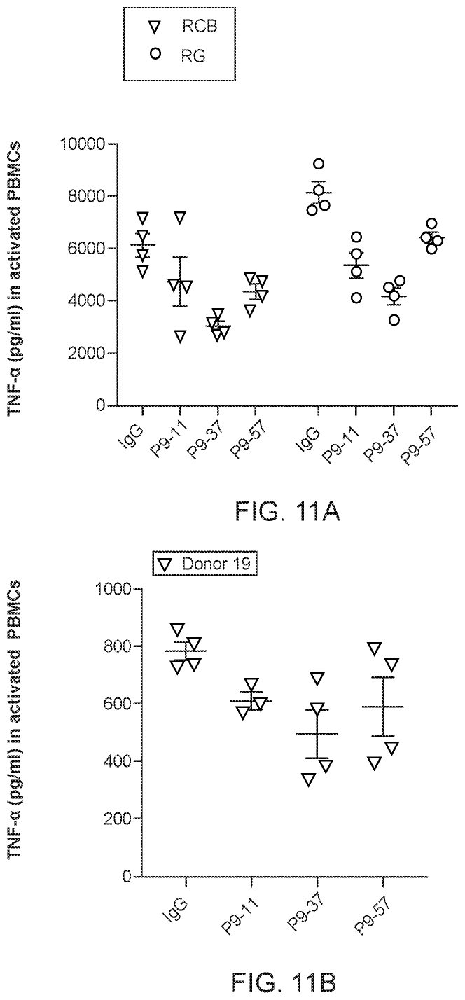

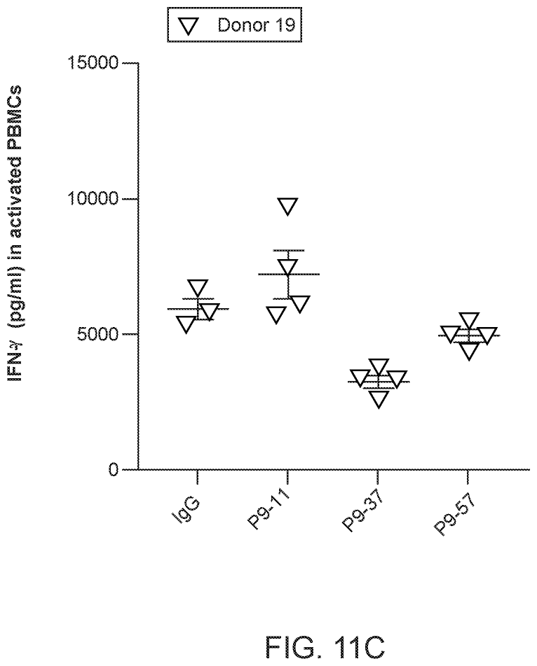

Representative data from 72 hrs of treatment are shown in FIGS. 11A-11C. The average is indicated as a horizontal bar on the scatter plots. Error bars show standard deviation.

FIGS. 11A-11B show scatter plots of TNF-α levels after with treatment with human IgG control (hIgG) and inhibitory anti-human GAL9 candidates. Treatment with P9-11, P9-37, or P9-57 formatted as human IgG1 antibodies, decreased TNF-α levels in PBMCs from all three human donors compared to IgG control. FIG. 11C show scatter plots of IFN-γ levels after treatment with a human control IgG (hIgG) or the anti-human GAL9 candidates. Treatment with either P9-11, P9-37, or P9-57 decreased IFN-γ levels in PBMCs as compared to control.

Treatment with either P9-41, P9-42, or P9-53 gave neutral or weak TNF-α and IFN-γ secretion (data not shown).

### 6.11.11. Example 10: Treating with Anti-Human GAL9 P9-11, P9-24, or P9-34 Decreases TNF-α and INF-γ Production and Increases IL-10 Production in Activated PBMCs

This study was conducted to determine the effect of select inhibitory anti-human GAL9 candidates from Example 7 on secretion of TNF-α, INF-γ, and IL-10 in activated human PBMCs.

Stimulation of PBMCs

PBMCs were stimulated essentially as described in Section 6.11.1 above. Briefly, PBMCs were harvested from human donors known to be highly responsive to human CMV virus (HCMV), placed in culture, stimulated with HCMV PepMix to prime an antigen specific response, and treated with one of P9-11, P9-24, and P9-34, formatted as a bivalent, monospecific, human IgG1 antibody, or a human IgG control.

Cytokine Assay

Cytokine secretion of TNF-α, INF-γ, and IL-10 was measured 72 hrs post-treatment using BD™ Cytometric Bead Array (CBA) following manufacturer's instructions.

Results/Conclusion

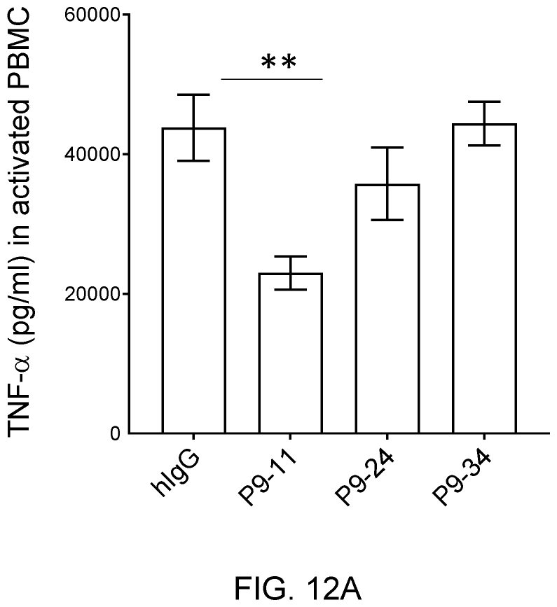

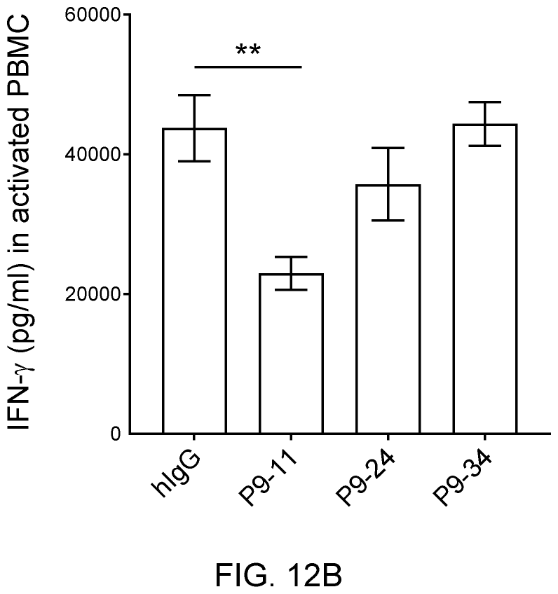

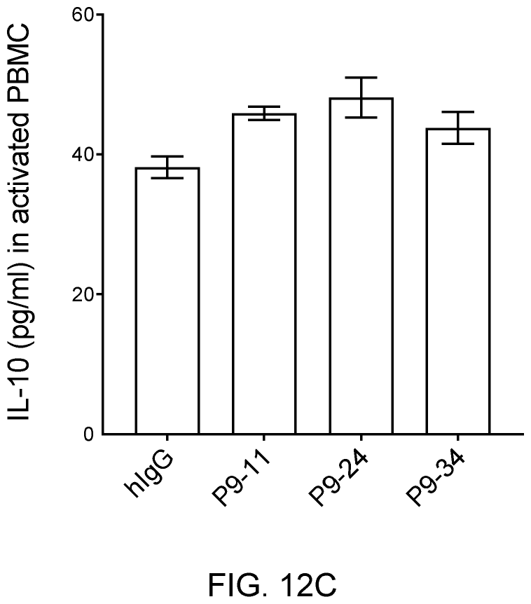

FIG. 12A shows bar graphs of TNF-α levels after treatment with control IgG (hIgG) or inhibitory anti-human GAL9 candidates. Treatment with anti-human IgG1 P9-11, P9-24, or P9-34 resulted in a decrease of TNF-α secretion from PBMCs compared to IgG control. FIG. 12B shows bar graphs of INF-γ levels after with treatment with control IgG (hIgG) or inhibitory anti-GAL9 candidates. Treatment with anti-human GAL9 antibodies P9-11, P9-24, or P9-34 resulted in a decrease of INF-γ secretion from PBMCs compared to IgG control. FIG. 12C shows bar graphs of IL-10 levels after with treatment inhibitory anti-human GAL9 candidates or IgG control. Treatment with P9-11, P9-24, or P9-34 antibodies increased IL-10 secretion in PBMCs as compared to control.

### 6.11.12. Example 11: Treating Activated CD3⁺ T-Cells with Anti-Human GAL9 Antibodies P9-11, P9-24, or P9-34 Improves the Cytokine Profile, while Anti-Mouse GAL9 (108A2) Results in a Complete Block of Cytokine Secretion

We measured INF-γ, TNF-α, or IL-10 cytokine secretion to determine the effect of anti-mouse GAL9 (clone 108A2) and anti-human GAL9 antibodies P9-11, P9-24, or P9-34, formatted as human IgG1 antibodies, on the cytokine profile in activated CD3⁺ T-cells from mice.

Animals and Isolation of CD3⁺ T-Cells

Five mice were used for each treatment group. All animals used in the study were housed and cared for in accordance with the NHMRC Guidelines for Animal Use.

Antibodies

Antibodies P9-11, P9-24, and P9-34, formatted as bivalent monospecific human IgG1 antibodies, and a human IgG control were used. In addition, the inhibitory anti-mouse GAL9 clone 108A2 “mGAL9” (BioLegend® San Diego, Calif.) was used.

Simulation of CD3⁺ T-Cells

CD3⁺ T-cells (CD90.2±CD3±) were isolated from the spleens of naïve mice. Mouse CD3⁺ T cells were stimulated with anti-CD3 clone 145.2C11 (Aviva Systems Biology Corp. San Diego, Calif.) at 5 μg/ml. Next, the stimulated CD3⁺ T cells were treated either with IgG control or one of the inhibitory antibodies at 20 μg/ml and cultured for 72 hours.

Cytokine Assays

After 72 hrs of treatment, the concentration of INF-γ, TNF-α, or IL-10 was measured using BD™ Cytometric Bead Array (CBA) following the manufacturer's instructions.

Statistical Analyses

Non-parametric unpaired t-test was conducted using GraphPad Prism (GraphPad Software).

Results/Conclusion

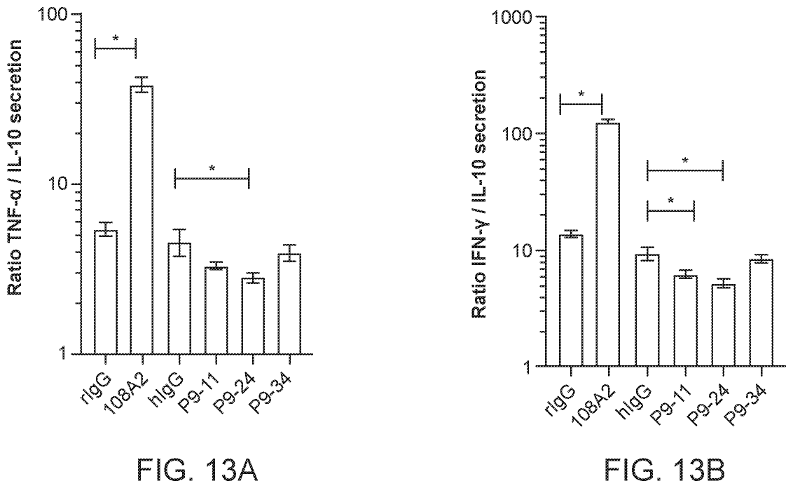

The results are shown in FIGS. 13A and 13B. A reduced ratio of TNF-α:IL-10 or INF-γ:IL:10 indicates a reduction in pro-inflammatory cytokines with an increase in the inhibitory cytokine, IL-10. Treatment with the anti-mouse GAL9 (108A2) antibody significantly reduced secretion of TNF-α, INF-γ, and IL-10. See FIG. 13A. In contrast, treatment with either anti-human GAL9 antibody P9-11, P9-24, or P9-34 (human IgG1 Fc) did not reduce TNF-α or INF-γ secretion, and IL-10 secretion was significantly increased. See FIG. 13B. The asterisk “*” indicates a statistical significance of p-value <0.05 compared to control.

Treatment with anti-human P9-11 and P9-24 antibodies, formatted as human IgG1 antibodies, resulted in an improved inflammatory environment, decreasing secretion of TNF-α, INF-γ, an increasing IL-10 secretion. Notably, treatment with anti-mouse GAL9 (108A2) resulted in a complete block of cytokine response, including IL-10 secretion. The differences in the cytokine profiles generated by anti-human GAL9 and anti-murine GAL9 (108A2) suggest that anti-human GAL9 and anti-mouse GAL9 (108A2) antibodies have a different mechanism of action.

### 6.11.13. Example 12: Treating with Anti-Human GAL9 does not Substantially Change the Expression of Immune Checkpoint Molecules in Stimulated CD4⁺ and CD8⁺ T Cells, and Decreases 4-1BB, CD40L, and OX40 Costimulatory Molecules in CD8⁺ T Cells

This study was conducted to determine the effect of anti-human GAL9 candidates P9-11, P9-24, and P9-34 on the expression of select checkpoint molecules in stimulated CD8⁺ and CD4⁺ T cells and the effect of anti-human GAL9 P9-11 on select costimulatory molecules in stimulated CD8⁺ T cells.

Stimulation & Treatment

PBMCs, which include the population of CD8⁺ or CD4⁺ T-cells, were stimulated as described above and treated with anti-human GAL9 P9-11, P9-24, P9-34, formatted as bivalent monospecific human IgG1 antibodies, or a human IgG control.

Immunolabelling

PMBCs were resuspended at 5×10⁶ cells/mL in 10% FBS in RPMI. 200 μL of resuspended cells were aliquoted to 96 well plates, then stained with Fixable Viability Dye eFluor® 780 for 30 minutes at 2-8° C. to irreversibly label dead cells. Cells were then washed and incubated with human Fc Block solution (Cat. No. 14-9161-73, eBiosciences) for 10 minutes at room temperature. The surface expression of PD-L1, PD-1, CTLA-4, TIM3, LAGS, 4-1BB, CD27, CD40L, ICOS, or OX40 was assessed by flow cytometry.

Flow Cytometry

Flow cytometry analysis was performed using a BD LSR Fortessa flow cytometer and BD FACSDiva software (Becton, Dickinson and Company, Franklin Lakes, N.J., USA). For each sample, at least 5×10⁵ events were collected.

Representative data for the percentage of CD4⁺ or CD8⁺ T-cells that stained positive for immune checkpoint molecules are presented in Table 11 and Table 12 below. Data for the percentage of CD8⁺ T-cells that stained positive for costimulatory molecules are presented in Table 13 below.

The “% value” represents the % of cells with detectable levels of the indicated marker. “(x)” indicates the fold change after treatment with the selected α-GAL9 antibody candidates as compared to a human IgG control.

TABLE 11
Percent CD4⁺ cells positive for selected immune checkpoint molecules

| Marker | PD-L1 | PD-1 | GAL9 | CTLA-4 | TIM3 | LAG3 |
|---|---|---|---|---|---|---|
| hIgG | 43.6% | 14.2% | 3.02% | 0.67% | 0.99% | 1.00% |
| Control | | | | | | |
| P9-11 | 37.3% (0.9x) | 14.2% (1.0x) | 2.21% (0.7x) | 0.71% (1.0x) | 1.14 % (1.1x) | 0.93% (0.9x) |
| P9-24 | 40.2% (0.9x) | 15.0% (1.0x) | 2.05% (0.6x) | 0.67% (1.0x) | 0.93% (0.9x) | 1.03% (1.0x) |
| P9-34 | 42.3% (0.9x) | 16.0% (1.1x) | 2.63% (0.8x) | 0.71% (1.0x) | 1.03% (1.0x) | 1.12% (1.1x) |

TABLE 12
Percent CD8⁺ cells positive for selected immune checkpoint molecules

| Marker | PD-L1 | PD-1 | GAL9 | CTLA-4 | TIM3 | LAG3 |
|---|---|---|---|---|---|---|
| hIgG | 29.1% | 16.1% | 4.35% | 18.7% | 0.81% | 2.25% |
| Control | | | | | | |
| P9-11 | 26.7% (0.9x) | 16.5% (1.0x) | 1.63% (0.3x) | 15.2% (0.8x) | 0.95% (1.1x) | 2.00% (0.9x) |
| P9-24 | 24.5% (0.8x) | 16.7% (1.0x) | 1.82% (0.4x) | 15.1% (0.8x) | 0.88% (1.0x) | 1.88% (0.8x) |
| P9-34 | 26.3% (0.9x) | 17.0% (1.0x) | 2.79% (0.6x) | 15.0% (0.8x) | 0.82% (1.0x) | 2.40% (1.0x) |

TABLE 13
Percent CD8⁺ cells positive for selected costimulatory molecules

| Marker | 4-1BB | CD27 | CD40L | ICOS | OX40 |
|---|---|---|---|---|---|
| hIgG | 5.64% | 53.5% | 2.57% | 6.39% | 9.95% |
| control | | | | | |
| P9-11 | 3.03% | 52.6% | 1.85% | 5.56% | 5.2% |
| | (0.53×) | (0.98×) | (0.72×) | (0.87×) | (0.5×) |

Results/Conclusion

There was no substantial change in the expression of any of the immune checkpoint molecules in stimulated CD8⁺ or CD4⁺ T-cells. However, we observed a decrease in the costimulatory molecules 4-1BB, CD40L, and OX40 in stimulated CD8⁺ T-cells. These results suggest that the effects of the anti-human GAL9 candidates on cytokine response is driven by the inhibition of GAL9, and not through PD-1/PD-L1 immune checkpoint pathway or other checkpoint molecules such as CTLA-4, TIM3, or LAGS.

### 7. EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

What is claimed is:

1. A Galectin-9 (GAL9) antigen binding molecule, comprising: a first antigen binding site (ABS) specific for a first epitope of a first GAL9 antigen, wherein the first antigen binding site comprises all three VH CDRs from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

2. A Galectin-9 (GAL9) antigen binding molecule, comprising a first antigen binding site (ABS) specific for a first epitope of a first GAL9 antigen, wherein the first antigen binding site comprises all three VL CDRs from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

3. A Galectin-9 (GAL9) antigen binding molecule, comprising a first antigen binding site (ABS) specific for a first epitope of a first GAL9 antigen, wherein the first antigen binding site comprises all three VH CDRs and all three VL CDRs from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

4. A Galectin-9 (GAL9) antigen binding molecule, comprising a first antigen binding site (ABS) specific for a first epitope of a first GAL9 antigen, comprising the VL sequence and the VH sequence from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

5. The GAL9 antigen binding molecule of claim 4, wherein the first antigen binding site (ABS) further comprises a first IgG heavy chain polypeptide and a first light chain polypeptide.

6. The GAL9 antigen binding molecule of any one of claims 1-5, wherein the GAL9 antigen is a human GAL9 antigen.

7. The GAL9 antigen binding molecule of any of claims 1-6, wherein the GAL9 antigen binding molecule further comprises a second antigen binding site (ABS).

8. The GAL9 antigen binding molecule of claim 7, wherein the second ABS is specific for a GAL9 antigen.

9. The GAL9 antigen binding molecule of claim 7, wherein the second ABS is specific for a second epitope of the first GAL9 antigen.

10. The GAL9 antigen binding molecule of claim 7, wherein the second ABS is specific for the first epitope of the first GAL9 antigen and is identical to the first ABS.

11. The GAL9 antigen binding molecule of any one of claims 7-10, wherein the second ABS comprises all three VH CDRs, all three VL CDRs, or all three VH CDRs and all three VL CDRs from another ABS clone selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

12. The GAL9 antigen binding molecule of claim 11, wherein the second antigen binding site comprises the VL sequence and the VH sequence from the other ABS clone.

13. The GAL9 antigen binding molecule of claim 12, wherein the second antigen binding site comprises a full immunoglobulin heavy chain sequence comprising the VH sequence and a full immunoglobulin light chain sequence comprising the VL sequence from the other ABS clone.

14. The GAL9 antigen binding molecule of claim 7, wherein the second antigen binding site is specific for an antigen other than the first GAL9 antigen.

15. The GAL9 antigen binding molecule of any one of the preceding claims, wherein the first antigen binding site comprises all three VH CDRs, all three VL CDRs, or all three VH CDRs and all three VL CDRs from any one of the ABS clones selected from: P9-11, P9-24, P9-34, and P9-37.

16. The GAL9 antigen binding molecule of any of claims 1-14, wherein the first antigen binding site comprises all three VH CDRs, all three VL CDRs, or all three VH CDRs and all three VL CDRs from any one of the ABS clones selected from P9-11, P9-24, and P9-34.

17. The GAL9 antigen binding molecule of any of claims 1-14, wherein the first antigen binding site comprises all three VH CDRs, all three VL CDRs, or all three VH CDRs and all three VL CDRs from ABS clone P9-11.

18. The GAL9 antigen binding molecule of any of claims 1-14, wherein the first antigen binding site comprises all three VH CDRs, all three VL CDRs, or all three VH CDRs and all three VL CDRs from ABS clone P9-24.

19. The GAL9 antigen binding molecule of any of claims 1-14, wherein the first antigen binding site comprises all three VH CDRs, all three VL CDRs, or all three VH CDRs and all three VL CDRs from ABS clone P9-34.

20. The GAL9 antigen binding molecule of any of claims 1-14, wherein the first antigen binding site comprises all three VH CDRs, all three VL CDRs, or all three VH CDRs and all three VL CDRs from ABS clone P9-37.

21. The GAL9 antigen binding molecule of any of claims 1-20, wherein the GAL9 antigen binding molecule comprises an antibody format selected from the group consisting of: full-length antibodies, Fab fragments, Fvs, scFvs, tandem scFvs, Diabodies, scDiabodies, DARTs, tandAbs, minibodies, and B-bodies.

22. The GAL9 antigen binding molecule of any of claims 1-21, wherein the GAL9 antigen binding molecule decreases TNF-α secretion by activated immune cells upon contact, wherein the decrease is about at least a 30%, 35%, 40%, 45%, 50%, 55%, or 60% decrease, relative to activated immune cells treated with a control agent.

23. The GAL9 antigen binding molecule of any of claims 1-22, wherein the GAL9 antigen binding molecule decreases IFN-γ secretion by activated immune cells upon contact, wherein the decrease is about at least a 20%, 25%, 30%, 35%, 40%, 45%, or 50% decrease relative to activated immune cells treated with a control agent.

24. The GAL9 antigen binding molecule of any of claims 1-23, wherein the GAL9 antigen binding molecule increases IL-10 secretion by activated immune cells upon contact, wherein the increase is about at least a 5%, 10%, 15%, 20%, 25%, 30%, 35% or 40% increase relative to activated immune cells treated with a control agent.

25. The GAL9 antigen binding molecule of any of claims 1-24, wherein the GAL9 antigen binding molecule does not modulate PD-1 surface expression on activated immune cells relative to activated immune cells treated with a control agent.

26. The GAL9 antigen binding molecule of any of claims 1-25, wherein the GAL9 antigen binding molecule does not modulate PD-L1 surface expression on activated immune cells relative to activated immune cells treated with a control agent.

27. The GAL9 antigen binding molecule of any of claims 1-26, wherein the GAL9 antigen binding molecule does not modulate CTLA-4 surface expression on activated immune cells relative to activated immune cells treated with a control agent.

28. The GAL9 antigen binding molecule of any of claims 1-27, wherein the GAL9 antigen binding molecule does not modulate TIM3 surface expression on activated immune cells relative to activated immune cells treated with a control agent.

29. The GAL9 antigen binding molecule of any of claims 1-28, wherein the GAL9 antigen binding molecule does not modulate LAG3 surface expression on activated immune cells relative to activated immune cells treated with a control agent.

30. The GAL9 antigen binding molecule of any of claims 1-29, wherein the GAL9 antigen binding molecule decreases 4-1BB surface expression on CD8⁺ T-cells, relative to CD8⁺ T-cells treated with a control agent.

31. The GAL9 antigen binding molecule of any of claims 1-30, wherein the GAL9 antigen binding molecule decreases CD40L surface expression on CD8⁺ T-cells, relative to CD8⁺ T-cells treated with a control agent.

32. The GAL9 antigen binding molecule of any of claims 1-31, wherein the GAL9 antigen binding molecule decreases OX40 surface expression on CD8⁺ T-cells, relative to CD8⁺ T-cells treated with a control agent.

33. The GAL9 antigen binding molecule of any of claims 22-32, wherein the control agent is a negative control agent or positive control agent.

34. The GAL9 antigen binding molecule of claim 33, wherein the control agent is a control antibody.

35. The GAL9 antigen binding molecule of claim 34, wherein the control antibody is selected from the group consisting of: an ECA42 clone anti-GAL9 antibody, an RG9.1 clone anti-GAL9 antibody, an RG9.35 clone anti-GAL9 antibody, an anti-PD1 antibody, a 108A2 clone anti-GAL9 antibody, and a non-GAL9 binding isotype control antibody.

36. The GAL9 antigen binding molecule of any one of claims 22-35, wherein the activated immune cells were activated by peptide stimulation, anti-CD3, or dendritic cells.

37. A GAL9 antigen binding molecule, wherein the GAL9 antigen binding molecule decreases TNF-α secretion by activated immune cells upon contact, wherein the decrease is about at least a 30%, 35%, 40%, 45%, 50%, 55%, or 60% decrease relative to activated immune cells treated with a control agent.

38. A GAL9 antigen binding molecule, wherein the GAL9 antigen binding molecule decreases IFN-γ secretion by activated immune cells upon contact, wherein the decrease is about at least a 20%, 25%, 30%, 35%, 40%, 45%, or 50% decrease relative to activated immune cells treated with a control agent.

39. A GAL9 antigen binding molecule, wherein the GAL9 antigen binding molecule increases IL-10 secretion by activated immune cells upon contact, wherein the increase is about at least a 5%, 10%, 15%, 20%, 25%, 30%, 35% or 40% increase relative to activated immune cells treated with a control agent

40. A GAL9 antigen binding molecule, wherein the GAL9 antigen binding molecule does not modulate PD-1 surface expression on activated immune cells relative to activated immune cells treated with a control agent.

41. A GAL9 antigen binding molecule, wherein the GAL9 antigen binding molecule does not modulate PD-L1 surface expression on activated immune cells relative to activated immune cells treated with a control agent.

42. A GAL9 antigen binding molecule, wherein the GAL9 antigen binding molecule does not modulate CTLA-4 surface expression on activated immune cells relative to activated immune cells treated with a control agent.

43. A GAL9 antigen binding molecule, wherein the GAL9 antigen binding molecule does not modulate TIM3 surface expression on activated immune cells relative to activated immune cells treated with a control agent.

44. A GAL9 antigen binding molecule, wherein the GAL9 antigen binding molecule does not modulate LAG-3 surface expression on activated immune cells relative to activated immune cells treated with a control agent.

45. A GAL9 antigen binding molecule decreases 4-1BB surface expression on activated CD8⁺ T-cells relative to activated CD8⁺ T-cells treated with a control agent.

46. A GAL9 antigen binding molecule decreases CD40L surface expression on activated CD8⁺ T-cells relative to activated CD8⁺ T-cells treated with a control agent.

47. A GAL9 antigen binding molecule decreases OX40 surface expression on activated CD8⁺ T-cells relative to activated CD8⁺ T-cells treated with a control agent.

48. A GAL9 antigen binding molecule, wherein the GAL9 antigen binding molecule demonstrates one or more of the following properties:
A) decreases TNF-α secretion by activated immune cells, wherein the decrease is about at least a 30%, 35%, 40%, 45%, 50%, 55%, or 60% decrease relative to activated immune cells treated with a control agent;
B) decreases IFN-γ secretion by activated immune cells, wherein the decrease is about at least a 20%, 25%, 30%, 35%, 40%, 45%, or 50% decrease relative to activated immune cells treated with a control agent;
C) increases IL-10 secretion by activated immune cells, wherein the increase is about at least a 5%, 10%, 15%, 20%, 25%, 30%, 35%, or 40% increase relative to activated immune cells treated with a control agent;
D) does not modulate PD-1 surface expression on activated immune cells relative to activated immune cells treated with a control agent;
E) does not modulate PD-L1 surface expression on activated immune cells relative to activated immune cells treated with a control agent;
F) does not modulate CTLA-4 surface expression on activated immune cells relative to activated immune cells treated with a control agent;
G) does not modulate TIM3 surface expression on activated immune cells relative to activated immune cells treated with a control agent;
H) does not modulate LAG3 surface expression on activated immune cells relative to activated immune cells treated with a control agent;
I) decreases 4-1BB surface expression on activated CD8⁺ T-cells relative to activated CD8⁺ T-cells treated with a control agent;
J) decreases CD40L surface expression on activated CD8⁺ T-cells relative to activated CD8⁺ T-cells treated with a control agent; or
K) decreases OX40 surface expression on activated CD8⁺ T-cells relative to activated CD8⁺ T-cells treated with a control agent.

49. The GAL9 antigen binding molecule of any one of claims 37-48, wherein the control agent is a negative control agent or positive control agent.

50. The GAL9 antigen binding molecule of claim 49, wherein the control agent is a control antibody.

51. The GAL9 antigen binding molecule of claim 50, wherein the control antibody is selected from the group consisting of: an ECA42 clone anti-GAL9 antibody, an RG9.1 clone anti-GAL9 antibody, an RG9.35 clone anti-GAL9 antibody, an anti-PD1 antibody, an 108A2 clone anti-GAL9 antibody, and an non-GAL9 binding isotype control antibody.

52. The GAL9 antigen binding molecule of any one of claims 37-51, wherein the activated immune cells, were activated by were activated by peptide stimulation, anti-CD3 or dendritic cells.

53. The GAL9 antigen binding molecule of any of claims 37-49, comprising a first antigen binding site specific for a first epitope of a first GAL9 antigen, wherein the first antigen binding site comprises all three VH CDRs and all three VL CDRs from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

54. The GAL9 antigen binding molecule of claim 53, comprising the VL sequence and the VH sequence from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

55. The GAL9 antigen binding molecule of claim 54, comprising a full immunoglobulin heavy chain sequence comprising the VH sequence and a full immunoglobulin light chain sequence comprising the VL sequence, wherein the VH sequence and the VL sequence are from any one of the ABS clones selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

56. The GAL9 antigen binding molecule of any one of claims 37-55, wherein the GAL9 antigen is a human GAL9 antigen.

57. The GAL9 antigen binding molecule of any of claims 37-56, wherein the GAL9 antigen binding molecule further comprises a second antigen binding site.

58. The GAL9 antigen binding molecule of claim 57, wherein the second antigen binding site is specific for the GAL9 antigen.

59. The GAL9 antigen binding molecule of claim 58, wherein the second antigen binding site is identical to the first antigen binding site.

60. The GAL9 antigen binding molecule of claim 57, wherein the second antigen binding site is specific for a second epitope of the first GAL9 antigen.

61. The GAL9 antigen binding molecule of claim 60, wherein the second antigen binding site comprises all three VH CDRs and all three VL CDRs from another ABS clone selected from P9-01, P9-02A, P9-03, P9-06, P9-07, P9-11, P9-12, P9-14, P9-23, P9-24, P9-25, P9-29, P9-30, P9-34, P9-37, P9-38, P9-40, P9-41, P9-42, P9-43, P9-44, P9-45, P9-46, P9-50, P9-51, P9-52, P9-53, P9-56, and P9-57.

62. The GAL9 antigen binding molecule of claim 61, wherein the second antigen binding site comprises the VL sequence and the VH sequence from the other ABS clone.

63. The GAL9 antigen binding molecule of claim 62, wherein the second antigen binding site comprises a full immunoglobulin heavy chain sequence comprising the VH sequence and a full immunoglobulin light chain sequence comprising the VL sequence from the other ABS clone.

64. The GAL9 antigen binding molecule of claim 57, wherein the second antigen binding site is specific for an antigen other than the first GAL9 antigen.

65. The GAL9 antigen binding molecule of any of claims 53-64, wherein the first antigen binding site comprises all three VH CDRs and all three VL CDRs from any one of the ABS clones selected from: P9-11, P9-24, P9-34, and P9-37.

66. The GAL9 antigen binding molecule of any of claims 53-64, wherein the first antigen binding site comprises all three VH CDRs and all three VL CDRs from any one of the ABS clones selected from: P9-11, P9-24, and P9-34.

67. The GAL9 antigen binding molecule of any of claims 53-64, wherein the first antigen binding site comprises all three VH CDRs and all three VL CDRs from ABS clone P9-11.

68. The GAL9 antigen binding molecule of any of claims 53-64, wherein the first antigen binding site comprises all three VH CDRs and all three VL CDRs from ABS clone P9-24.

69. The GAL9 antigen binding molecule of any of claims 53-64, wherein the first antigen binding site comprises all three VH CDRs and all three VL CDRs from ABS clone P9-34.

70. The GAL9 antigen binding molecule of any of claims 53-64, wherein the first antigen binding site comprises all three VH CDRs and all three VL CDRs from ABS clone P9-37.

71. The GAL9 antigen binding molecule of any of claims 37-70, wherein the GAL9 antigen binding molecule comprises an antibody format selected from the group consisting of: full-length antibodies, Fab fragments, Fvs, scFvs, tandem scFvs, Diabodies, scDiabodies, DARTs, tandAbs, minibodies, and B-bodies.

72. A GAL9 antigen binding molecule which binds to the same epitope as a GAL9 antigen binding molecule of any one of the preceding claims.

73. A GAL9 antigen binding molecule which competes for binding with a GAL9 antigen binding molecule of any one of the preceding claims.

74. The GAL9 antigen binding molecule of any one of the preceding claims, which is purified.

75. A pharmaceutical composition comprising the GAL9 antigen binding molecule of any one of the preceding claims and a pharmaceutically acceptable diluent.

76. A method for treating a subject with an autoimmune disease, comprising: administering a therapeutically effective amount of the pharmaceutical composition of claim 75 to the subject.

77. The method of claim 76, wherein the subject with an autoimmune disease has increased PD-L2 expression on dendritic cells relative to dendritic cells from a healthy control.

78. The method of claim 76, wherein the autoimmune disease is selected from the group consisting of: inflammatory bowel disease, Crohn's disease, ulcerative colitis, colitis, celiac disease, rheumatoid arthritis, Behçet's disease, amyloidosis, psoriasis, psoriatic arthritis, systemic lupus erythematosus nephritis, graft-versus-host disease (GvHD), nonalcoholic steatohepatitis (NASH), and ankylosing spondylitis.

79. The method of claim 76, wherein the treatment results in reducing inflammation, reducing an autoimmune response, prolonging remission, inducing remission, re-establishing immune tolerance, improving organ function, reducing progression of a disease, reducing the risk of progression or development of a second disease, or increasing overall survival.
